SEQUENCE LISTING (77 sequences)

SEQ ID NO: 1 - protein, 5 aa, Mus sp.
DYSMH

SEQ ID NO: 2 - protein, 17 aa, Mus sp.
WINTETGEPTYADDFKG

SEQ ID NO: 3 - protein, 13 aa, Mus sp.
PYYDYVSYYAMDY

SEQ ID NO: 4 - protein, 122 aa, Mus sp.
QIQLVQSGPELKKPGETVKISCKASGYTFTDYSMHWVKQAPGKGLKWMGWINTETGEPTYADDFKGRFAFSLETSASTAYLQINNLKNEDTATYFCANPYYDYVSYYAMDYWGHGTSVTVSS

SEQ ID NO: 5 - protein, 122 aa, Artificial Sequence
  source: /note=“Description of Artificial Sequence Synthetic polypeptide”
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 6 - DNA, 458 bp, Artificial Sequence
  source: /note=“Description of Artificial Sequence Synthetic polynucleotide”
actagtaccaccatggcttgggtgtggaccttgctattcctgatggcagctgcccaaagtatccaagcacaggttcagttggtgcagtctggatctgagctgaagaagcctggagcctcagtcaaggtttcctgcaaggcttctggttataccttcacagactattcaatgcactgggtgcgacaggctccaggacaaggtttaaagtggatgggctggataaacactgagactggtgagccaacatatgcagatgacttcaagggacggtttgtcttctctttggacacctctgtcagcactgcctatttgcagatcagcagcctcaaagctgaggacacggctgtgtattactgtgctaatccctactatgattacgtctcttactatgctatggactactggggtcagggaaccacggtcaccgtctcctcaggtaagaatggcctctcaagctt

SEQ ID NO: 7 - protein, 11 aa, Mus sp.
KASQDVSTAVA

SEQ ID NO: 8 - protein, 7 aa, Mus sp.
SASYLYT

SEQ ID NO: 9 - protein, 9 aa, Mus sp.
QQHYSTPRT

SEQ ID NO: 10 - protein, 107 aa, Mus sp.
DIVMTQSHKFMSTSVRDRVSITCKASQDVSTAVAWYQQKPGQSPKLLIYSASYLYTGVPDRFTGSGSGTDFTFTISSVQAEDLAVYYCQQHYSTPRTFGGGTKLEIK

SEQ ID NO: 11 - protein, 107 aa, Artificial Sequence
  source: /note=“Description of Artificial Sequence Synthetic polypeptide”
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 12 - DNA, 416 bp, Artificial Sequence
  source: /note=“Description of Artificial Sequence Synthetic polynucleotide”
gctagcaccaccatggagtcacagattcaggtctttgtattcgtgtttctctggttgtctggtgttgacggagacattcagatgacccagtctccatcctccctgtccgcatcagtgggagacagggtcaccatcacctgcaaggccagtcaggatgtgagtactgctgtagcctggtatcaacagaaaccaggaaaagcccctaaactactgatttactcggcatcctacctctacactggagtcccttcacgcttcagtggcagtggatctgggacggatttcactttcaccatcagcagtctgcagcctgaagacattgcaacatattactgtcagcaacattatagtactcctcggacgttcggtcagggcaccaagctggaaatcaaacgtaagtagaatccaaagaattc

SEQ ID NO: 13 - protein, 5 aa, Mus sp.
SHDMS

SEQ ID NO: 14 - protein, 17 aa, Mus sp.
AINSDGGSTYYPDTMER

SEQ ID NO: 15 - protein, 11 aa, Mus sp.
HYDDYYAWFAY

SEQ ID NO: 16 - protein, 120 aa, Mus sp.
EVQLVESGGGLVQPGESLKLSCESNEYEFPSHDMSWVRKTPEKRLELVAAINSDGGSTYYPDTMERRFIISRDNTKKTLYLQMSSLRSEDTALYYCARHYDDYYAWFAYWGQGTLVTVSA

SEQ ID NO: 17 - protein, 120 aa, Artificial Sequence
  source: /note=“Description of Artificial Sequence Synthetic polypeptide”
EVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 18 - DNA, 451 bp, Artificial Sequence
  source: /note=“Description of Artificial Sequence Synthetic polynucleotide”
actagtaccaccatggacttcgggctcagcttggttttccttgtccttattttaaaaagtgtacagtgtgaggtgcagctggtggagtctgggggaggcttagtgcagcctggagggtccctgagactctcctgtgcagcctctgaatacgagttcccttcccatgacatgtcttgggtccgccaggctccggggaaggggctggagttggtcgcagccattaatagtgatggtggtagcacctactatccagacaccatggagagacgattcaccatctccagagacaatgccaagaactcactgtacctgcaaatgaacagtctgagggccgaggacacagccgtgtattactgtgcaagacactatgatgattactacgcctggtttgcttactggggccaagggactatggtcactgtctcttcaggtgagtcctaacttcaagctt

SEQ ID NO: 19 - protein, 15 aa, Mus sp.
RASKSVSTSGYSYMH

SEQ ID NO: 20 - protein, 7 aa, Mus sp.
LASNLES

SEQ ID NO: 21 - protein, 9 aa, Mus sp.
QHSRELPLT

SEQ ID NO: 22 - protein, 111 aa, Mus sp.
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELK

SEQ ID NO: 23 - protein, 111 aa, Artificial Sequence
  source: /note=“Description of Artificial Sequence Synthetic polypeptide”
EIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 24 - DNA, 428 bp, Artificial Sequence
  source: /note=“Description of Artificial Sequence Synthetic polynucleotide”
gctagcaccaccatggagacagacacactcctgttatgggtactgctgctctgggttccaggttccactggtgaaattgtgctgacacagtctcctgctaccttatctttgtctccaggggaaagggccaccctctcatgcagggccagcaaaagtgtcagtacatctggctatagttatatgcactggtaccaacagaaaccaggacaggctcccagactcctcatctatcttgcatccaacctagaatctggggtccctgccaggttcagtggcagtgggtctgggacagacttcaccctcaccatcagcagcctagagcctgaggattttgcagtttattactgtcagcacagtagggagcttccgctcacgttcggcggagggaccaaggtcgagatcaaacgtaagtacacttttctgaattc

SEQ ID NO: 25 - protein, 5 aa, Mus sp.
DAWMD

SEQ ID NO: 26 - protein, 19 aa, Mus sp.
EIRSKANNHATYYAESVNG

SEQ ID NO: 27 - protein, 8 aa, Mus sp.
GEVFYFDY

SEQ ID NO: 28 - DNA, 414 bp, Mus sp.
atgtacttgggactgaactatgtattcatagtttttctcttaaatggtgtccagagtgaagtgaagcttgaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctca

SEQ ID NO: 29 - protein, 138 aa, Mus sp.
MYLGLNYVFIVFLLNGVQSEVKLEESGGGLVQPGGSMKLSCAASGFTFSDAWMDWVRQSPEKGLEWVAEIRSKANNHATYYAESVNGRFTISRDDSKSSVYLQMNSLRAEDTGIYYCTWGEVFYFDYWGQGTTLTVSS

SEQ ID NO: 30 - DNA, 448 bp, Artificial Sequence
  source: /note=“Description of Artificial Sequence Synthetic polynucleotide”
actagtaccaccatgtacttgggactgaactatgtattcatagtttttctcttaaatggtgtccagagtgaagtgaagctggaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctcaggtgagtccttaaaacaagctt

SEQ ID NO: 31 - protein, 138 aa, Artificial Sequence; identical to SEQ ID NO: 29
  source: /note=“Description of Artificial Sequence Synthetic polypeptide”

SEQ ID NO: 32 - protein, 11 aa, Mus sp.
KSSQDINKYIA

SEQ ID NO: 33 - protein, 7 aa, Mus sp.
YTSTLQP

SEQ ID NO: 34 - protein, 8 aa, Mus sp.
LQYDNLLT

SEQ ID NO: 35 - DNA, 378 bp, Mus sp.
atgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaa

SEQ ID NO: 36 - protein, 126 aa, Mus sp.
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELK

SEQ ID NO: 37 - DNA, 413 bp, Artificial Sequence
  source: /note=“Description of Artificial Sequence Synthetic polynucleotide”
gctagcaccaccatgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaacgtaagtacacttttctgaattc

SEQ ID NO: 38 - protein, 126 aa, Artificial Sequence; identical to SEQ ID NO: 36
  source: /note=“Description of Artificial Sequence Synthetic polypeptide”

SEQ ID NO: 39 - DNA, 20 bp, Artificial Sequence
  source: /note=“Description of Artificial Sequence Synthetic primer”
cgctgttttgacctccatag

SEQ ID NO: 40 - DNA, 20 bp, Artificial Sequence
  source: /note=“Description of Artificial Sequence Synthetic primer”
tgaaagatgagctggaggac

SEQ ID NO: 41 - DNA, 20 bp, Artificial Sequence
  source: /note=“Description of Artificial Sequence Synthetic primer”
ctttcttgtccaccttggtg

SEQ ID NO: 42 - DNA, 19 bp, Artificial Sequence
  source: /note=“Description of Artificial Sequence Synthetic primer”
gctgtcctacagtcctcag

SEQ ID NO: 43 - DNA, 18 bp, Artificial Sequence
  source: /note=“Description of Artificial Sequence Synthetic primer”
acgtgccaagcatcctcg

SEQ ID NO: 44 - DNA, 1407 bp, Artificial Sequence
  source: /note=“Description of Artificial Sequence Synthetic polynucleotide”
atgtacttgggactgaactatgtattcatagtttttctcttaaatggtgtccagagtgaagtgaagctggaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 45 - protein, 469 aa, Artificial Sequence [sequence omitted]

SEQ ID NO: 46 - DNA, 702 bp, Artificial Sequence
  source: /note=“Description of Artificial Sequence Synthetic polynucleotide”
atgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 47 - protein, 233 aa, Artificial Sequence
  source: /note=“Description of Artificial Sequence Synthetic polypeptide”
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 452 aa, Artificial Sequence
  source: /note=“Description of Artificial Sequence Synthetic polypeptide”
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 214 aa, Artificial Sequence
  source: /note=“Description of Artificial Sequence Synthetic polypeptide”
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 448 aa, Artificial Sequence
  source: /note=“Description of Artificial Sequence Synthetic polypeptide”
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 215 aa, Artificial Sequence
  source: /note=“Description of Artificial Sequence Synthetic polypeptide”
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 118 aa, Artificial Sequence
  source: /note=“Description of Artificial Sequence Synthetic polypeptide”
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 53 - protein, 108 aa, Artificial Sequence
  source: /note=“Description of Artificial Sequence Synthetic polypeptide”
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 54 - protein, 5 aa, Artificial Sequence
  source: /note=“Description of Artificial Sequence Synthetic peptide”
SYTMH

SEQ ID NO: 55 - protein, 17 aa, Artificial Sequence
  source: /note=“Description of Artificial Sequence Synthetic peptide”
FISYDGNNKYYADSVKG

SEQ ID NO: 56 - protein, 9 aa, Artificial Sequence
  source: /note=“Description of Artificial Sequence Synthetic peptide”
TGWLGPFDY

SEQ ID NO: 57 - protein, 12 aa, Artificial Sequence
  source: /note=“Description of Artificial Sequence Synthetic peptide”
RASQSVGSSYLA

SEQ ID NO: 58 - protein, 7 aa, Artificial Sequence
  source: /note=“Description of Artificial Sequence Synthetic peptide”
GAFSRAT

SEQ ID NO: 59 - protein, 9 aa, Artificial Sequence
  source: /note=“Description of Artificial Sequence Synthetic peptide”
QQYGSSPWT

SEQ ID NO: 60 - protein, 451 aa, Artificial Sequence
  source: /note=“Description of Artificial Sequence Synthetic polypeptide”
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 61 - protein, 214 aa, Artificial Sequence
  source: /note=“Description of Artificial Sequence Synthetic polypeptide”
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 125 aa, Artificial Sequence
  source: /note=“Description of Artificial Sequence Synthetic polypeptide”
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSS

SEQ ID NO: 63 - protein, 107 aa, Artificial Sequence
  source: /note=“Description of Artificial Sequence Synthetic polypeptide”
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIK

SEQ ID NO: 64 - protein, 10 aa, Artificial Sequence
  source: /note=“Description of Artificial Sequence Synthetic peptide”
GFTFSSYGMH

SEQ ID NO: 65 - protein, 15 aa, Artificial Sequence
  source: /note=“Description of Artificial Sequence Synthetic peptide”
VIWYDGSNKYYADSV

SEQ ID NO: 66 - protein, 16 aa, Artificial Sequence
  source: /note=“Description of Artificial Sequence Synthetic peptide”
DPRGATLYYYYYGMDV

SEQ ID NO: 67 - protein, 11 aa, Artificial Sequence
  source: /note=“Description of Artificial Sequence Synthetic peptide”
RASQSINSYLD

SEQ ID NO: 68 - protein, 7 aa, Artificial Sequence
  source: /note=“Description of Artificial Sequence Synthetic peptide”
AASSLQS

SEQ ID NO: 69 - protein, 9 aa, Artificial Sequence
  source: /note=“Description of Artificial Sequence Synthetic peptide”
QQYYSTPFT

SEQ ID NO: 70 - protein, 131 aa, Homo sapiens
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARDSRGYSYYDFWSGYFYYYYMDVWGKGTTVTVSS

SEQ ID NO: 71 - protein, 113 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPPWASYTFGQGTKLEIKRT

SEQ ID NO: 72 - protein, 141 aa, Artificial Sequence
  source: /note=“Description of Artificial Sequence Synthetic polypeptide”
MAWVWTLLFLMAAAQSIQAQVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 73 - protein, 127 aa, Artificial Sequence
  source: /note=“Description of Artificial Sequence Synthetic polypeptide”
MESQIQVFVFVFLWLSGVDGDIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 74 - protein, 136 aa, Homo sapiens
GLSWVFLVAILEGVQCEVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARGLTGATDAFDIWGQGTMVTVSS

SEQ ID NO: 75 - protein, 128 aa, Homo sapiens
MEAPAQLLFLLLLWLPDTTGEIVLTQSPATLSLSPGERATLSCRASQSVSSYLAWYQQKPGQAPRLLIYDASNKATGVPARFSGSGSGTDFTLTISSLEPEDFAVYYCQQSSKWPLTFGGGTKVEIKG

SEQ ID NO: 76 - protein, 139 aa, Artificial Sequence
  source: /note=“Description of Artificial Sequence Synthetic polypeptide”
MDFGLSLVFLVLILKSVQCEVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 77 - protein, 131 aa, Artificial Sequence
  source: /note=“Description of Artificial Sequence Synthetic polypeptide”
METDTLLLWVLLLWVPGSTGEIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

(19) United States
(12) Patent Application Publication
(10) Pub. No.: US 2018/0222990 A1
(43) Pub. Date: Aug. 9, 2018

(54) Combination Treatments and Uses and Methods Thereof

(71) Applicant: GLAXOSMITHKLINE INTELLECTUAL PROPERTY DEVELOPMENT LIMITED, Brentford, Middlesex (GB)

(72) Inventors: Axel HOOS, Collegeville, PA (US); Niranjan YANAMANDRA, Collegeville, PA (US)

(73) Assignee: GLAXOSMITHKLINE INTELLECTUAL PROPERTY DEVELOPMENT LIMITED, Brentford, Middlesex (GB)

(21) Appl. No.: 15/749,553

(22) PCT Filed: Aug. 3, 2016
(86) PCT No.: PCT/IB2016/054698

§ 371 Date: Feb. 1, 2018

Related U.S. Application Data
(60) Provisional application No. 62/200,789, filed on Aug. 4, 2015.

(51) Int. Cl. C07K 16/28; A61P 35/00; A61K 39/395; A61K 45/06

(52) U.S. Cl. CPC C07K 16/2878; A61P 35/00; C07K 16/2818; A61K 2039/545; A61K 39/3955; A61K 45/06; A61K 39/39558

(57) ABSTRACT

Disclosed herein are combinations of an OX40 modulator and a CTLA-4 modulator, pharmaceutical compositions thereof, uses thereof, and methods of treatment comprising administering said combination, including uses in cancer.

### CROSS REFERENCE TO RELATED APPLICATIONS

This application claims priority to U.S. Application Ser. No. 62/200,789, filed on Aug. 4, 2015. The disclosure of the prior application is considered part of (and are incorporated by reference in) the disclosure of this application.

### SEQUENCE LISTING

The instant application contains a Sequence Listing which has been submitted electronically in ASCII format and is hereby incorporated by reference in its entirety. Said ASCII copy, created on Jul. 29, 2016, is named PU65947PCT_SL.txt and is 68,931 bytes in size.

### FIELD OF THE INVENTION

The present invention relates to a method of treating cancer in a mammal and to combinations useful in such treatment. In particular the present invention relates to combinations of anti-OX40 antigen binding proteins (ABPs), including monoclonal antibodies to human OX40 and one or more anti-CTLA-4 ABPs, including monoclonal antibodies to human CTLA-4.

### BACKGROUND OF THE INVENTION

Effective treatment of hyperproliferative disorders including cancer is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes include abnormalities in signal transduction pathways and response to factors which differ from those found in normal cells.

Immunotherapies are one approach to treat hyperproliferative disorders. A major hurdle that scientists and clinicians have encountered in the development of various types of cancer immunotherapies has been to break tolerance to self antigen (cancer) in order to mount a robust anti-tumor response leading to tumor regression. Unlike traditional development of small and large molecule agents that target the tumor, cancer immunotherapies target cells of the immune system that have the potential to generate a memory pool of effector cells to induce more durable effects and minimize recurrences.

OX40 is a costimulatory molecule involved in multiple processes of the immune system. Antigen binding proteins and antibodies that bind OX-40 receptor and modulate OX40 signalling are known in the art and are disclosed as immunotherapy, for example for cancer.

CTLA-4 is a negative regulator of T-cell activity. Blockade of CTLA-4 has been shown to augment T-cell activation and proliferation, including the activation and proliferation of tumor infiltrating T-effector cells. Inhibition of CTLA-4 signaling can also reduce T-regulatory cell function, which may contribute to a general increase in T cell responsiveness, including the anti-tumor immune response.

Enhancing anti-tumor T cell function and inducing T cell proliferation is a powerful and new approach for cancer treatment. Three immune-oncology antibodies (e.g., immuno-modulators) are presently marketed. Anti-CTLA-4 (YERVOY®/ipilimumab) is thought to augment immune responses at the point of T cell priming and anti-PD-1 antibodies (OPDIVO®/nivolumab and KEYTRUDA®/pembrolizumab) are thought to act in the local tumor microenvironment, by relieving an inhibitory checkpoint in tumor specific T cells that have already been primed and activated.

Though there have been many recent advances in the treatment of cancer, there remains a need for more effective and/or enhanced treatment of an individual suffering the effects of cancer. The combinations and methods herein that relate to combining therapeutic approaches for enhancing anti-tumor immunity address this need.

### SUMMARY OF THE INVENTION

The present invention provides methods of treating cancer in a mammal in need thereof comprising administering a therapeutically effective amount of an antigen binding protein that binds OX40 and an antigen binding protein that binds CTLA-4.

Also provided are pharmaceutical compositions comprising a therapeutically effective amount of an antigen binding protein that binds OX40 and a therapeutically effective amount of an antigen binding protein that binds CTLA-4. Suitably, kits are provided comprising the pharmaceutical compositions of the invention together with one or more pharmaceutically acceptable carriers.

Methods are provided for reducing tumor size in a human having cancer comprising administering a therapeutically effective amount of an agonist antibody to human OX-40 and a therapeutically effective amount of an antagonist antibody to human CTLA-4.

In some aspects, the disclosure provides a method of treating cancer in a mammal in need thereof comprising administering to the mammal a therapeutically effective amount of an antigen binding protein that binds OX40 and an antigen binding protein that binds CTLA-4.

In some embodiments, the cancer is a solid tumor. In some embodiments, the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

In some embodiments, the cancer is a liquid tumor.

In some embodiments, the antigen binding protein that binds OX40 and the antigen binding that binds CTLA-4 are administered at the same time.

In some embodiments, the antigen binding protein that binds OX40 and the antigen binding protein that binds CTLA-4 are administered sequentially, in any order.

In some embodiments, the antigen binding protein that binds OX40 and/or the antigen binding protein that binds CTLA-4 are administered systemically, e.g. intravenously.

In some embodiments, the antigen binding protein that binds OX40 and/or the antigen binding protein that binds CTLA-4 is administered intratumorally.

In some embodiments, the mammal is human.

In some embodiments, the tumor size of said cancer in said mammal is reduced by more than an additive amount compared with treatment with the antigen binding protein to OX-40 and the antigen binding protein to CTLA-4 as used as monotherapy.

In some embodiments, the antigen binding protein that binds OX40 binds to human OX40.

In some embodiments, in the antigen binding protein that binds to CTLA-4 binds to human CTLA-4.

In some embodiments, the antigen binding protein that binds OX40 and/or the antigen binding protein that binds CTLA-4 is a humanized monoclonal antibody.

In some embodiments, the antigen binding protein that binds OX40 and/or the antigen binding protein that binds CTLA-4 is a fully human monoclonal antibody.

In some embodiments, the antigen binding protein that binds OX40 and/or the antigen binding protein that binds CTLA-4 is an antibody with an IgG1 antibody isotype or variant thereof.

In some embodiments, the antigen binding protein that binds OX40 and/or the antigen binding protein that binds CTLA-4 is an antibody with an IgG4 antibody isotype or variant thereof.

In some embodiments, the antigen binding protein that binds OX40 is an agonist antibody.

In some embodiments, the antigen binding protein that binds CTLA-4 is an antagonist antibody.

In some embodiments, the antigen binding protein that binds OX40 comprises: a heavy chain variable region CDR1 comprising an amino acid sequence with at least 90%, 91%, 92%, 93%, 94%, 95%, 96%, 97%, 98%, 99% or 100% sequence identity to the amino acid sequence set forth in SEQ ID NO:1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO:2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:3 or 15.

In some embodiments, the antigen binding protein that binds OX40 comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO:7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO:8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:9 or 21.

In some embodiments, the antigen binding protein that binds OX40 comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the antigen binding protein that binds OX40 comprises: (a) a heavy chain variable region CDR1 comprising the amino acid sequence of SEQ ID NO:13; (b) a heavy chain variable region CDR2 comprising the amino acid sequence of SEQ ID NO:14;

(c) a heavy chain variable region CDR3 comprising the amino acid sequence of SEQ ID NO:15; (d) a light chain variable region CDR1 comprising the amino acid sequence of SEQ ID NO:19; (e) a light chain variable region CDR2 comprising the amino acid sequence of SEQ ID NO:20; and (f) a light chain variable region CDR3 comprising the amino acid sequence of SEQ ID NO:21.

In some embodiments, the antigen binding protein that binds OX40 comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:10, 11, 22 or 23.

In some embodiments, the antigen binding protein that binds OX40 comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:4, 5, 16 and 17.

In some embodiments, the antigen binding protein that binds OX40 comprises a heavy chain variable region comprising the amino acid sequence set forth in SEQ ID NO:5 and a light chain variable region comprising the amino acid sequence set forth in SEQ ID NO:11.

In some embodiments, the antigen binding protein that binds OX40 comprises a heavy chain variable region comprising the amino acid sequence set forth in SEQ ID NO:17 and a light chain variable region comprising the amino acid sequence set forth in SEQ ID NO:23.

In some embodiments, the antigen binding protein that binds OX40 comprises a light chain variable region comprising the amino acid sequence of SEQ ID NO:11 or 23, or an amino acid sequence with at least 90% sequence identity to the amino acid sequences of SEQ ID NO:11 or 23.

In some embodiments, the antigen binding protein that binds OX40 comprises a heavy chain variable region comprising the amino acid sequence of SEQ ID NO:5 or 17, or an amino acid sequence with at least 90% sequence identity to the amino acid sequences of SEQ ID NO:5 or 17.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the antigen binding protein that binds CTLA-4 is ipilimumab, or an antibody comprising 90%, 91%, 92%, 93%, 94%, 95%, 96%, 97%, 98%, 99% or 100% sequence identity thereto, e.g., to the HC CDRs, LC CDRs, VH, VL, HC and/or LC thereof.

In some embodiments, the antigen binding protein that binds CTLA-4 is tremelimumab, or an antibody having 90%, 91%, 92%, 93%, 94%, 95%, 96%, 97%, 98%, 99% or 100% sequence identity thereto, e.g., the HC CDRs, LC CDRs, VH, VL, HC and/or LC thereof.

In some embodiments, the mammal has increased survival when treated with a therapeutically effective amount of an antigen binding protein to OX-40 and therapeutically effective amount of an antigen binding protein to CTLA-4 compared with a mammal who received the antigen binding protein to OX-40 or the antigen binding protein to CTLA-4 as monotherapy.

In some embodiments, the method further comprises administering at least one anti-neoplastic agent to the mammal in need thereof.

In some aspects, the disclosure provides a pharmaceutical composition or kit comprising a therapeutically effective amount of an antigen binding protein that binds OX40 and a therapeutically effective amount of an antigen binding protein that binds CTLA-4.

In some embodiments, the pharmaceutical composition or kit as described herein comprises an antibody comprising an antigen binding protein that binds OX40 comprising a heavy chain variable region CDR1 having an amino acid sequence with at least 90%, 91%, 92%, 93%, 94%, 95%, 96%, 97%, 98%, 99% or 100% sequence identity to the amino acid sequence as set forth in SEQ ID NO:1, a heavy chain variable region CDR2 having an amino acid sequence with at least 90%, 91%, 92%, 93%, 94%, 95%, 96%, 97%, 98%, 99% or 100% sequence identity to the amino acid sequence as set forth in SEQ ID NO:2, a heavy chain variable region CDR3 having an amino acid sequence with at least 90%, 91%, 92%, 93%, 94%, 95%, 96%, 97%, 98%, 99% or 100% sequence identity to the amino acid sequence as set forth in SEQ ID NO:3, a light chain variable region CDR1 having an amino acid sequence with at least 90%, 91%, 92%, 93%, 94%, 95%, 96%, 97%, 98%, 99% or 100% sequence identity to the amino acid sequence as set forth in SEQ ID NO:7, a light chain variable region CDR2 having an amino acid sequence with at least 90%, 91%, 92%, 93%, 94%, 95%, 96%, 97%, 98%, 99% or 100% sequence identity to the amino acid sequence as set forth in SEQ ID NO:8, a light chain variable region CDR3 having an amino acid sequence with at least 90%, 91%, 92%, 93%, 94%, 95%, 96%, 97%, 98%, 99% or 100% sequence identity to the amino acid sequence as set forth in SEQ ID NO:9; and ipilimumab, or an antibody comprising 90%, 91%, 92%, 93%, 94%, 95%, 96%, 97%, 98%, 99% or 100% sequence identity thereto, e.g., to the HC CDRs, LC CDRs, VH, VL, HC and/or LC thereof.

In some aspects, the disclosure provides a pharmaceutical composition or kit as described herein, comprising an antibody comprising a VH region having a sequence at least with a sequence at least 90%, 91%, 92%, 93%, 94%, 95%, 96%, 97%, 98%, 99% or 100% sequence identity to the amino acid sequence as set forth in SEQ ID NO:4 or 5 and VL having a sequence at least with a sequence at least 90%, 91%, 92%, 93%, 94%, 95%, 96%, 97%, 98%, 99% or 100% sequence identity to the amino acid sequence as set forth in SEQ ID NO:10 or 11, and ipilimumab, or an antibody comprising 90%, 91%, 92%, 93%, 94%, 95%, 96%, 97%, 98%, 99% or 100% sequence identity thereto, e.g., to the HC CDRs, LC CDRs, VH, VL, HC and/or LC thereof.

In some embodiments, the pharmaceutical composition or kit as described herein, comprising an antibody comprising an antigen binding protein that binds OX40 comprising a heavy chain variable region CDR1 having an amino acid sequence with at least 90%, 91%, 92%, 93%, 94%, 95%, 96%, 97%, 98%, 99% or 100% sequence identity to the amino acid sequence as set forth in SEQ ID NO:1, a heavy chain variable region CDR2 having an amino acid sequence with at least 90%, 91%, 92%, 93%, 94%, 95%, 96%, 97%, 98%, 99% or 100% sequence identity to the amino acid sequence as set forth in SEQ ID NO:2, a heavy chain variable region CDR3 having an amino acid sequence with at least 90%, 91%, 92%, 93%, 94%, 95%, 96%, 97%, 98%, 99% or 100% sequence identity to the amino acid sequence as set forth in SEQ ID NO:3, a light chain variable region CDR1 having an amino acid sequence with at least 90%, 91%, 92%, 93%, 94%, 95%, 96%, 97%, 98%, 99% or 100% sequence identity to the amino acid sequence as set forth in SEQ ID NO:7, a light chain variable region CDR2 having an amino acid sequence with at least 90%, 91%, 92%, 93%, 94%, 95%, 96%, 97%, 98%, 99% or 100% sequence identity to the amino acid sequence as set forth in SEQ ID NO:8, a light chain variable region CDR3 having an amino acid sequence with at least 90%, 91%, 92%, 93%, 94%, 95%, 96%, 97%, 98%, 99% or 100% sequence identity to the amino acid sequence as set forth in SEQ ID NO:9; and tremelimumab, or an antibody comprising 90%, 91%, 92%, 93%, 94%, 95%, 96%, 97%, 98%, 99% or 100% sequence identity thereto, e.g., to the HC CDRs, LC CDRs, VH, VL, HC and/or LC thereof.

In some aspects, the disclosure provides a pharmaceutical composition or kit as described herein, comprising an antibody comprising a VH region having a sequence at least with a sequence at least 90%, 91%, 92%, 93%, 94%, 95%, 96%, 97%, 98%, 99% or 100% sequence identity to the amino acid sequence as set forth in SEQ ID NO:4 or 5 and VL having a sequence at least with a sequence at least 90%, 91%, 92%, 93%, 94%, 95%, 96%, 97%, 98%, 99% or 100% sequence identity to the amino acid sequence as set forth in SEQ ID NO:10 or 11, and tremelimumab, or an antibody comprising 90%, 91%, 92%, 93%, 94%, 95%, 96%, 97%, 98%, 99% or 100% sequence identity thereto, e.g., to the HC CDRs, LC CDRs, VH, VL, HC and/or LC thereof.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some aspects, the disclosure provides use of a combination or pharmaceutical composition or kit as described herein in the manufacture of a medicament for the treatment of cancer.

In some aspects, the disclosure provides a combination kit comprising a pharmaceutical composition or kit as described herein together with one or more pharmaceutically acceptable carriers.

In some aspects, the disclosure provides a method of reducing tumor size in a human having cancer comprising administering a therapeutically effective amount of an agonist antibody to human OX-40 and a therapeutically effective amount of an antagonist antibody to human CTLA-4, e.g., as described herein.

In some aspects, the disclosure provides a kit for use in the treatment of cancer comprising:
a therapeutically effective amount of an antigen binding protein that binds OX40 and an antigen binding protein that binds CTLA-4, and
instructions for use in the treatment of cancer.

In some embodiments, the cancer is a solid tumor. In some embodiments, the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

In some embodiments, the cancer is a liquid tumor.

In some embodiments, the antigen binding protein that binds OX40 binds to human OX40.

In some embodiments, the antigen binding protein that binds to CTLA-4 binds to human CTLA-4.

In some embodiments, the antigen binding protein that binds OX40 and/or the antigen binding protein that binds CTLA-4 is a humanized monoclonal antibody.

In some embodiments, the antigen binding protein that binds OX40 and/or the antigen binding protein that binds CTLA-4 is a fully human monoclonal antibody.

In some embodiments, the antigen binding protein that binds OX40 and/or the antigen binding protein that binds CTLA-4 is an antibody with an IgG1 antibody isotype or variant thereof.

In some embodiments, the antigen binding protein that binds OX40 and/or the antigen binding protein that binds CTLA-4 is an antibody with an IgG4 antibody isotype or variant thereof.

In some embodiments, the antigen binding protein that binds OX40 is an agonist antibody.

In some embodiments, the antigen binding protein that binds CTLA-4 is an antagonist antibody.

In some embodiments, the antigen binding protein that binds OX40 comprises: a heavy chain variable region CDR1 comprising an amino acid sequence with at least 90%, 91%, 92%, 93%, 94%, 95%, 96%, 97%, 98%, 99% or 100% sequence identity to the amino acid sequence set forth in SEQ ID NO:1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO:2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:3 or 15.

In some embodiments, the antigen binding protein that binds OX40 comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO:7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO:8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:9 or 21.

In some embodiments, the antigen binding protein that binds OX40 comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the antigen binding protein that binds OX40 comprises: (a) a heavy chain variable region CDR1 comprising the amino acid sequence of SEQ ID NO:13; (b) a heavy chain variable region CDR2 comprising the amino acid sequence of SEQ ID NO:14; (c) a heavy chain variable region CDR3 comprising the amino acid sequence of SEQ ID NO:15; (d) a light chain variable region CDR1 comprising the amino acid sequence of SEQ ID NO:19; (e) a light chain variable region CDR2 comprising the amino acid sequence of SEQ ID NO:20; and (f) a light chain variable region CDR3 comprising the amino acid sequence of SEQ ID NO:21.

In some embodiments, the antigen binding protein that binds OX40 comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:10, 11, 22 or 23.

In some embodiments, the antigen binding protein that binds OX40 comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:4, 5, 16 and 17.

In some embodiments, the antigen binding protein that binds OX40 comprises a heavy chain variable region comprising the amino acid sequence set forth in SEQ ID NO:5 and a light chain variable region comprising the amino acid sequence set forth in SEQ ID NO:11.

In some embodiments, the antigen binding protein that binds OX40 comprises a heavy chain variable region comprising the amino acid sequence set forth in SEQ ID NO:17 and a light chain variable region comprising the amino acid sequence set forth in SEQ ID NO:23.

In some embodiments, the antigen binding protein that binds OX40 comprises a light chain variable region comprising the amino acid sequence of SEQ ID NO:11 or 23, or an amino acid sequence with at least 90% sequence identity to the amino acid sequences of SEQ ID NO:11 or 23.

In some embodiments, the antigen binding protein that binds OX40 comprises a heavy chain variable region comprising the amino acid sequence of SEQ ID NO:5 or 17, or an amino acid sequence with at least 90% sequence identity to the amino acid sequences of SEQ ID NO:5 or 17.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the antigen binding protein that binds CTLA-4 is ipilimumab, or an antibody comprising 90%, 91%, 92%, 93%, 94%, 95%, 96%, 97%, 98%, 99% or 100% sequence identity thereto, e.g., to the HC CDRs, LC CDRs, VH, VL, HC and/or LC thereof.

In some embodiments, the antigen binding protein that binds CTLA-4 is tremelimumab, or an antibody having 90%, 91%, 92%, 93%, 94%, 95%, 96%, 97%, 98%, 99% or 100% sequence identity thereto, e.g., the HC CDRs, LC CDRs, VH, VL, HC and/or LC thereof.

In some embodiments, the kit further comprises at least one anti-neoplastic agent.

In some aspects, the disclosure provides a therapeutically effective amount of an antigen binding protein that binds OX40 and an antigen binding protein that binds CTLA-4 for use (e.g., simultaneous or sequential use) in treating cancer in a mammal in need thereof.

In some embodiments, the cancer is a solid tumor. In some embodiments, the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

In some embodiments, the cancer is a liquid tumor.

In some embodiments, the antigen binding protein that binds OX40 and the antigen binding that binds CTLA-4 are to be administered at the same time.

In some embodiments, the antigen binding protein that binds OX40 and the antigen binding protein that binds CTLA-4 are to be administered sequentially, in any order.

In some embodiments, the antigen binding protein that binds OX40 and/or the antigen binding protein that binds CTLA-4 are to be administered systemically, e.g. intravenously.

In some embodiments, the antigen binding protein that binds OX40 and/or the antigen binding protein that binds CTLA-4 is to be administered intratumorally.

In some embodiments, the mammal is human.

In some embodiments, the tumor size of said cancer in said mammal is reduced by more than an additive amount compared with treatment with the antigen binding protein to OX-40 and the antigen binding protein to CTLA-4 as used as monotherapy.

In some embodiments, the antigen binding protein that binds OX40 binds to human OX40. In some embodiments, the antigen binding protein that binds to CTLA-4 binds to human CTLA-4.

In some embodiments, the antigen binding protein that binds OX40 and/or the antigen binding protein that binds CTLA-4 is a humanized monoclonal antibody.

In some embodiments, the antigen binding protein that binds OX40 and/or the antigen binding protein that binds CTLA-4 is a fully human monoclonal antibody.

In some embodiments, the antigen binding protein that binds OX40 and/or the antigen binding protein that binds CTLA-4 is an antibody with an IgG1 antibody isotype or variant thereof.

In some embodiments, the antigen binding protein that binds OX40 and/or the antigen binding protein that binds CTLA-4 is an antibody with an IgG4 antibody isotype or variant thereof.

In some embodiments, the antigen binding protein that binds OX40 is an agonist antibody.

In some embodiments, the antigen binding protein that binds CTLA-4 is an antagonist antibody.

In some embodiments, the antigen binding protein that binds OX40 comprises: a heavy chain variable region CDR1 comprising an amino acid sequence with at least 90%, 91%, 92%, 93%, 94%, 95%, 96%, 97%, 98%, 99% or 100% sequence identity to the amino acid sequence set forth in SEQ ID NO:1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO:2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:3 or 15.

In some embodiments, the antigen binding protein that binds OX40 comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO:7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO:8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:9 or 21.

In some embodiments, the antigen binding protein that binds OX40 comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the antigen binding protein that binds OX40 comprises: (a) a heavy chain variable region CDR1 comprising the amino acid sequence of SEQ ID NO:13; (b) a heavy chain variable region CDR2 comprising the amino acid sequence of SEQ ID NO:14; (c) a heavy chain variable region CDR3 comprising the amino acid sequence of SEQ ID NO:15; (d) a light chain variable region CDR1 comprising the amino acid sequence of SEQ ID NO:19; (e) a light chain variable region CDR2 comprising the amino acid sequence of SEQ ID NO:20; and (f) a light chain variable region CDR3 comprising the amino acid sequence of SEQ ID NO:21.

In some embodiments, the antigen binding protein that binds OX40 comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:10, 11, 22 or 23.

In some embodiments, the antigen binding protein that binds OX40 comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:4, 5, 16 and 17.

In some embodiments, the antigen binding protein that binds OX40 comprises a heavy chain variable region comprising the amino acid sequence set forth in SEQ ID NO:5 and a light chain variable region comprising the amino acid sequence set forth in SEQ ID NO:11.

In some embodiments, the antigen binding protein that binds OX40 comprises a heavy chain variable region comprising the amino acid sequence set forth in SEQ ID NO:17 and a light chain variable region comprising the amino acid sequence set forth in SEQ ID NO:23.

In some embodiments, the antigen binding protein that binds OX40 comprises a light chain variable region comprising the amino acid sequence of SEQ ID NO:11 or 23, or an amino acid sequence with at least 90% sequence identity to the amino acid sequences of SEQ ID NO:11 or 23.

In some embodiments, the antigen binding protein that binds OX40 comprises a heavy chain variable region comprising the amino acid sequence of SEQ ID NO:5 or 17, or an amino acid sequence with at least 90% sequence identity to the amino acid sequences of SEQ ID NO:5 or 17.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the antigen binding protein that binds CTLA-4 is ipilimumab, or an antibody comprising 90%, 91%, 92%, 93%, 94%, 95%, 96%, 97%, 98%, 99% or 100% sequence identity thereto, e.g., the HC CDRs, LC CDRs, VH, VL, HC and/or LC thereof.

In some embodiments, the antigen binding protein that binds CTLA-4 is tremelimumab, or an antibody having 90%, 91%, 92%, 93%, 94%, 95%, 96%, 97%, 98%, 99% or 100% sequence identity thereto, e.g., the HC CDRs, LC CDRs, VH, VL, HC and/or LC thereof.

In some embodiments, the mammal has increased survival when treated with a therapeutically effective amount of an antigen binding protein to OX-40 and therapeutically effective amount of an antigen binding protein to CTLA-4 compared with a mammal who received the antigen binding protein to OX-40 or the antigen binding protein to CTLA-4 as monotherapy.

In some embodiments, the antigen binding proteins are for use with at least one anti-neoplastic agent.

In some aspects, the disclosure provides a therapeutically effective amount of an agonist antibody to human OX-40 and a therapeutically effective amount of an antagonist antibody to human CTLA-4 for use (e.g., simultaneous or sequential use) in reducing tumor size in a human having cancer.

In some aspects, the disclosure provides use (e.g., simultaneous or sequential use) of a therapeutically effective amount of an antigen binding protein that binds OX40 and an antigen binding protein that binds CTLA-4 for the preparation of a medicament for treating cancer in a mammal in need thereof.

In some embodiments, the cancer is a solid tumor. In some embodiments, the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

In some embodiments, the cancer is a liquid tumor.

In some embodiments, the antigen binding protein that binds OX40 and the antigen binding that binds CTLA-4 are administered at the same time.

In some embodiments, the antigen binding protein that binds OX40 and the antigen binding protein that binds CTLA-4 are administered sequentially, in any order.

In some embodiments, the antigen binding protein that binds OX40 and/or the antigen binding protein that binds CTLA-4 are administered systemically, e.g. intravenously.

In some embodiments, the antigen binding protein that binds OX40 and/or the antigen binding protein that binds CTLA-4 is administered intratumorally.

In some embodiments, the mammal is human.

In some embodiments, the tumor size of said cancer in said mammal is reduced by more than an additive amount compared with treatment with the antigen binding protein to OX-40 and the antigen binding protein to CTLA-4 as used as monotherapy.

In some embodiments, the antigen binding protein that binds OX40 binds to human OX40.

In some embodiments, the antigen binding protein that binds to CTLA-4 binds to human CTLA-4.

In some embodiments, the antigen binding protein that binds OX40 and/or the antigen binding protein that binds CTLA-4 is a humanized monoclonal antibody.

In some embodiments, the antigen binding protein that binds OX40 and/or the antigen binding protein that binds CTLA-4 is a fully human monoclonal antibody.

In some embodiments, the antigen binding protein that binds OX40 and/or the antigen binding protein that binds CTLA-4 is an antibody with an IgG1 antibody isotype or variant thereof.

In some embodiments, the antigen binding protein that binds OX40 and/or the antigen binding protein that binds CTLA-4 is an antibody with an IgG4 antibody isotype or variant thereof.

In some embodiments, the antigen binding protein that binds OX40 is an agonist antibody.

In some embodiments, the antigen binding protein that binds CTLA-4 is an antagonist antibody.

In some embodiments, the antigen binding protein that binds OX40 comprises: a heavy chain variable region CDR1 comprising an amino acid sequence with at least 90%, 91%, 92%, 93%, 94%, 95%, 96%, 97%, 98%, 99% or 100% sequence identity to the amino acid sequence set forth in SEQ ID NO:1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO:2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:3 or 15.

In some embodiments, the antigen binding protein that binds OX40 comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO:7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO:8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:9 or 21.

In some embodiments, the antigen binding protein that binds OX40 comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the antigen binding protein that binds OX40 comprises: (a) a heavy chain variable region CDR1 comprising the amino acid sequence of SEQ ID NO:13; (b) a heavy chain variable region CDR2 comprising the amino acid sequence of SEQ ID NO:14; (c) a heavy chain variable region CDR3 comprising the amino acid sequence of SEQ ID NO:15; (d) a light chain variable region CDR1 comprising the amino acid sequence of SEQ ID NO:19; (e) a light chain variable region CDR2 comprising the amino acid sequence of SEQ ID NO:20; and (f) a light chain variable region CDR3 comprising the amino acid sequence of SEQ ID NO:21.

In some embodiments, the antigen binding protein that binds OX40 comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:10, 11, 22 or 23.

In some embodiments, the antigen binding protein that binds OX40 comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:4, 5, 16 and 17.

In some embodiments, the antigen binding protein that binds OX40 comprises a heavy chain variable region comprising the amino acid sequence set forth in SEQ ID NO:5 and a light chain variable region comprising the amino acid sequence set forth in SEQ ID NO:11.

In some embodiments, the antigen binding protein that binds OX40 comprises a heavy chain variable region comprising the amino acid sequence set forth in SEQ ID NO:17 and a light chain variable region comprising the amino acid sequence set forth in SEQ ID NO:23.

In some embodiments, the antigen binding protein that binds OX40 comprises a light chain variable region comprising the amino acid sequence of SEQ ID NO:11 or 23, or an amino acid sequence with at least 90% sequence identity to the amino acid sequences of SEQ ID NO:11 or 23.

In some embodiments, the antigen binding protein that binds OX40 comprises a heavy chain variable region comprising the amino acid sequence of SEQ ID NO:5 or 17, or an amino acid sequence with at least 90% sequence identity to the amino acid sequences of SEQ ID NO:5 or 17.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the antigen binding protein that binds CTLA-4 is ipilimumab, or an antibody comprising 90%, 91%, 92%, 93%, 94%, 95%, 96%, 97%, 98%, 99% or 100% sequence identity thereto, e.g., the HC CDRs, LC CDRs, VH, VL, HC and/or LC thereof.

In some embodiments, the antigen binding protein that binds CTLA-4 is tremelimumab, or an antibody having 90%, 91%, 92%, 93%, 94%, 95%, 96%, 97%, 98%, 99% or 100% sequence identity thereto, e.g., the HC CDRs, LC CDRs, VH, VL, HC and/or LC thereof.

In some embodiments, the mammal has increased survival when treated with a therapeutically effective amount of an antigen binding protein to OX-40 and therapeutically effective amount of an antigen binding protein to CTLA-4 compared with a mammal who received the antigen binding protein to OX-40 or the antigen binding protein to CTLA-4 as monotherapy.

In some embodiments, the use further comprises at least one anti-neoplastic agent for administration to the mammal in need thereof.

In some aspects, the disclosure provides use of a therapeutically effective amount of an agonist antibody to human OX-40 and a therapeutically effective amount of an antagonist antibody to human CTLA-4 for the preparation of a medicament for reducing tumor size in a human having cancer.

In some aspects, the disclosure provides a method for increasing IFNg protein or IFNg mRNA levels (e.g., determined as described herein) in a mammal, the method comprising:

administering to the mammal a therapeutically effective amount of an antigen binding protein that binds OX40 and an antigen binding protein that binds CTLA-4, wherein the antigen binding protein that binds OX40 and the antigen binding protein that binds CTLA-4 are as described herein, and, e.g., administered as described herein. E.g., wherein the mammal (e.g., human) has cancer, as described herein. Also provided herein is a therapeutically effective amount of an antigen binding protein that binds OX40 and an antigen binding protein that binds CTLA-4, both as described herein, for use in increasing IFNg protein or mRNA levels in the mammal. Also provided herein is use of a therapeutically effective amount of an antigen binding protein that binds OX40 and an antigen binding protein that binds CTLA-4, both as described herein, for the preparation of a medicament for increasing IFNg protein or mRNA levels in the mammal.

In some aspects, the disclosure provides a method for increasing IFNg protein or IFNg mRNA levels (e.g., determined as described herein) in a mammal, the method comprising:

administering to the mammal a therapeutically effective amount of an antigen binding protein that binds OX40, wherein the antigen binding protein that binds OX40 is as described herein, and, e.g., administered as described herein. E.g., wherein the mammal (e.g., human) has cancer, as described herein. Also provided herein is a therapeutically effective amount of an antigen binding protein that binds OX40, as described herein, for use in increasing IFNg protein or mRNA levels in the mammal. Also provided herein is use of a therapeutically effective amount of an antigen binding protein that binds OX40, as described herein, for the preparation of a medicament for increasing IFNg protein or mRNA levels in the mammal.

In some aspects, the disclosure provides a method for increasing TNF-a protein or TNF-a mRNA levels (e.g., determined as described herein) in a mammal, the method comprising:

administering to the mammal a therapeutically effective amount of an antigen binding protein that binds OX40 and an antigen binding protein that binds CTLA-4, wherein the antigen binding protein that binds OX40 and the antigen binding protein that binds CTLA-4 are as described herein, and, e.g., administered as described herein. E.g., wherein the mammal (e.g., human) has cancer, as described herein. Also provided herein is a therapeutically effective amount of an antigen binding protein that binds OX40 and an antigen binding protein that binds CTLA-4, both as described herein, for use in increasing TNF-a protein or mRNA levels in the mammal. Also provided herein is use of a therapeutically effective amount of an antigen binding protein that binds OX40 and an antigen binding protein that binds CTLA-4, both as described herein, for the preparation of a medicament for increasing TNF-a protein or mRNA levels in the mammal.

In some aspects, the disclosure provides a method for increasing TNF-a protein or TNF-a mRNA levels (e.g., determined as described herein) in a mammal, the method comprising:

administering to the mammal a therapeutically effective amount of an antigen binding protein that binds OX40, wherein the antigen binding protein that binds OX40 is as described herein, and, e.g., administered as described herein. E.g., wherein the mammal (e.g., human) has cancer, as described herein. Also provided herein is a therapeutically effective amount of an antigen binding protein that binds OX40, as described herein, for use in increasing TNF-a protein or mRNA levels in the mammal. Also provided herein is use of a therapeutically effective amount of an antigen binding protein that binds OX40, as described herein, for the preparation of a medicament for increasing TNF-a protein or mRNA levels in the mammal.

In some aspects, the disclosure provides a method for increasing IL-6 protein or IL-6 mRNA levels (e.g., determined as described herein) in a mammal, the method comprising:

administering to the mammal a therapeutically effective amount of an antigen binding protein that binds OX40 and an antigen binding protein that binds CTLA-4, wherein the antigen binding protein that binds OX40 and the antigen binding protein that binds CTLA-4 are as described herein, and, e.g., administered as described herein. E.g., wherein the mammal (e.g., human) has cancer, as described herein. Also provided herein is a therapeutically effective amount of an antigen binding protein that binds OX40 and an antigen binding protein that binds CTLA-4, both as described herein, for use in increasing IL-6 protein or mRNA levels in the mammal. Also provided herein is use of a therapeutically effective amount of an antigen binding protein that binds OX40 and an antigen binding protein that binds CTLA-4, both as described herein, for the preparation of a medicament for increasing IL-6 protein or mRNA levels in the mammal.

In some aspects, the disclosure provides a method for increasing IL-6 protein or IL-6 mRNA levels (e.g., determined as described herein) in a mammal, the method comprising:

administering to the mammal a therapeutically effective amount of an antigen binding protein that binds OX40, wherein the antigen binding protein that binds OX40 is as described herein, and, e.g., administered as described herein. E.g., wherein the mammal (e.g., human) has cancer, as described herein. Also provided herein is a therapeutically effective amount of an antigen binding protein that binds OX40, as described herein, for use in increasing IL-6 protein or mRNA levels in the mammal. Also provided herein is use of a therapeutically effective amount of an antigen binding protein that binds OX40, as described herein, for the preparation of a medicament for increasing IL-6 protein or mRNA levels in the mammal.

In some aspects, the disclosure provides a method for increasing ICOS protein or ICOS mRNA levels (e.g., determined as described herein) in the spleen of a mammal, the method comprising:

administering to the mammal a therapeutically effective amount of an antigen binding protein that binds OX40 and an antigen binding protein that binds CTLA-4, wherein the antigen binding protein that binds OX40 and the antigen binding protein that binds CTLA-4 are as described herein, and, e.g., administered as described herein. E.g., wherein the mammal (e.g., human) has cancer, as described herein. Also provided herein is a therapeutically effective amount of an antigen binding protein that binds OX40 and an antigen binding protein that binds CTLA-4, both as described herein, for use in increasing ICOS protein or mRNA levels in the spleen of the mammal. Also provided herein is use of a therapeutically effective amount of an antigen binding protein that binds OX40 and an antigen binding protein that binds CTLA-4, both as described herein, for the preparation of a medicament for increasing ICOS protein or mRNA levels in the spleen of the mammal.

In some aspects, the disclosure provides a method for increasing PD-1 protein or PD-1 mRNA levels (e.g., determined as described herein) in the spleen of a mammal, the method comprising:

administering to the mammal a therapeutically effective amount of an antigen binding protein that binds OX40 and an antigen binding protein that binds CTLA-4, wherein the antigen binding protein that binds OX40 and the antigen binding protein that binds CTLA-4 are as described herein, and, e.g., administered as described herein. E.g., wherein the mammal (e.g., human) has cancer, as described herein. Also provided herein is a therapeutically effective amount of an antigen binding protein that binds OX40 and an antigen binding protein that binds CTLA-4, both as described herein, for use in increasing PD-1 protein or mRNA levels in the spleen of the mammal. Also provided herein is use of a therapeutically effective amount of an antigen binding protein that binds OX40 and an antigen binding protein that binds CTLA-4, both as described herein, for the preparation of a medicament for increasing PD-1 protein or mRNA levels in the spleen of the mammal.

In some aspects, the disclosure provides a method for increasing CD4+ and/or CD8+ T cell proliferation (e.g., as determined by Ki67+ staining) in the spleen in a mammal, the method comprising:

administering to the mammal a therapeutically effective amount of an antigen binding protein that binds OX40 and an antigen binding protein that binds CTLA-4, wherein the antigen binding protein that binds OX40 and the antigen binding protein that binds CTLA-4 are as described herein and, e.g., administered as described herein. E.g., wherein the mammal (e.g., human) has cancer, as described herein. Also provided herein is a therapeutically effective amount of an antigen binding protein that binds OX40 and an antigen binding protein that binds CTLA-4, both as described herein, for use in increasing CD4+ and/or CD8+ T cell proliferation in the spleen of the mammal. Also provided herein is use of a therapeutically effective amount of an antigen binding protein that binds OX40 and an antigen binding protein that binds CTLA-4, both as described herein, for the preparation of a medicament for increasing CD4+ and/or CD8+ T cell proliferation in the spleen of the mammal.

In some aspects, the disclosure provides a method for increasing CD4+ T cell proliferation (e.g., as determined by Ki67+ staining) in the spleen in a mammal, the method comprising:

administering to the mammal a therapeutically effective amount of an antigen binding protein that binds OX40, wherein the antigen binding protein that binds OX40 is as described herein and, e.g., administered as described herein. E.g., wherein the mammal (e.g., human) has cancer, as described herein. Also provided herein is a therapeutically effective amount of an antigen binding protein that binds OX40, as described herein, for use in increasing CD4+ T cell proliferation in the spleen of the mammal. Also provided herein is use of a therapeutically effective amount of an antigen binding protein that binds OX40, as described herein, for the preparation of a medicament for increasing CD4+ T cell proliferation in the spleen of the mammal.

In some aspects, the disclosure provides a method for increasing granzyme B levels in CD8+ T cells (e.g., determined as described herein) in a tumor in a mammal, the method comprising:

administering to the mammal a therapeutically effective amount of an antigen binding protein that binds OX40 and an antigen binding protein that binds CTLA-4, wherein the antigen binding protein that binds OX40 and the antigen binding protein that binds CTLA-4 are as described herein and, e.g., administered as described herein. E.g., wherein the mammal (e.g., human) has cancer, as described herein. Also provided herein is a therapeutically effective amount of an antigen binding protein that binds OX40 and an antigen binding protein that binds CTLA-4, both as described herein, for use in increasing granzyme B levels in CD8+ T cells in a tumor in the mammal. Also provided herein is use of a therapeutically effective amount of an antigen binding protein that binds OX40 and an antigen binding protein that binds CTLA-4, both as described herein, for the preparation of a medicament for increasing granzyme B levels in CD8+ T cells in a tumor in the mammal.

In some aspects, the disclosure provides a method for increasing granzyme B levels in CD8+ T cells (e.g., determined as described herein) in a tumor in a mammal, the method comprising:

administering to the mammal a therapeutically effective amount of an antigen binding protein that binds OX40, wherein the antigen binding protein that binds is as described herein and, e.g., administered as described herein. E.g., wherein the mammal (e.g., human) has cancer, as described herein. Also provided herein is a therapeutically effective amount of an antigen binding protein that binds OX40, as described herein, for use in increasing granzyme B levels in CD8+ T cells in a tumor in the mammal. Also provided herein is use of a therapeutically effective amount of an antigen binding protein that binds OX40, as described herein, for the preparation of a medicament for increasing granzyme B levels in CD8+ T cells in a tumor in the mammal.

In some aspects, the disclosure provides a method for increasing the CD8:Treg cell ratio (e.g., determined as described herein) in a tumor in a mammal, the method comprising:

administering to the mammal a therapeutically effective amount of an antigen binding protein that binds OX40 and an antigen binding protein that binds CTLA-4, wherein the antigen binding protein that binds OX40 and the antigen binding protein that binds CTLA-4 are as described herein and, e.g., administered as described herein. E.g., wherein the mammal (e.g., human) has cancer, as described herein. Also provided herein is a therapeutically effective amount of an antigen binding protein that binds OX40 and an antigen binding protein that binds CTLA-4, both as described herein, for use in increasing CD8:Treg cell ratio in a tumor in the mammal. Also provided herein is use of a therapeutically effective amount of an antigen binding protein that binds OX40 and an antigen binding protein that binds CTLA-4, both as described herein, for the preparation of a medicament for increasing CD8:Treg cell ratio in a tumor in the mammal.

In some aspects, the disclosure provides a method for increasing clonality of T cells (e.g., determined as described herein) in blood and/or tumor in a mammal, the method comprising:

administering to the mammal a therapeutically effective amount of an antigen binding protein that binds OX40 and an antigen binding protein that binds CTLA-4, wherein the antigen binding protein that binds OX40 and the antigen binding protein that binds CTLA-4 are as described herein and, e.g., administered as described herein. E.g., wherein the mammal (e.g., human) has cancer, as described herein. Also provided herein is a therapeutically effective amount of an antigen binding protein that binds OX40 and an antigen binding protein that binds CTLA-4, both as described herein, for use in increasing clonality of T cells in blood and/or tumor in the mammal. Also provided herein is use of a therapeutically effective amount of an antigen binding protein that binds OX40 and an antigen binding protein that binds CTLA-4, both as described herein, for the preparation of a medicament for increasing clonality of T cells in blood and/or tumor in the mammal.

In some aspects, the disclosure provides a method for increasing clonality of T cells (e.g., determined as described herein) in a tumor in a mammal, the method comprising:

administering to the mammal a therapeutically effective amount of an antigen binding protein that binds OX40, wherein the antigen binding protein that binds OX40 is as described herein and, e.g., administered as described herein. E.g., wherein the mammal (e.g., human) has cancer, as described herein. Also provided herein is a therapeutically effective amount of an antigen binding protein that binds OX40, as described herein, for use in increasing clonality of T cells in a tumor in the mammal. Also provided herein is use of a therapeutically effective amount of an antigen binding protein that binds OX40, as described herein, for the preparation of a medicament for increasing clonality of T cells in a tumor in the mammal.

### BRIEF DESCRIPTION OF THE DRAWINGS

FIGS. 1-12 show sequences of the anti-OX40 ABPs of a combination of the invention, or a method or use thereof, e.g. CDRs and VH and VL sequences.

FIG. 13 is a series of panels showing line graphs showing tumor measurements (mm³) over time with the indicated treatments, as listed in panels a)-j).

FIG. 14 is a line graph showing survival after the indicated treatments.

FIG. 15A, FIG. 15B and FIG. 15C. FIG. 15A is a series of panels of tumor growth curves showing tumor volume (mm³) over time with the indicated treatments. FIG. 15B is a line graph showing survival after the indicated treatments. FIG. 15C is a line graph showing tumor measurements (mm³) over time after rechallenge.

FIG. 16 is a series of three bar graphs showing IFN-g, TNF-a, and IL-6 serum cytokine levels after the indicated treatments, as measured by MSD.

FIG. 17A and FIG. 17B are bar graphs showing percent CD4+Ki67+ cells (FIG. 17A) and percent CD8+Ki67+ cells (FIG. 17B) after the indicated treatments.

FIG. 18A and FIG. 18B are bar graphs showing percent CD8+ ICOS+ cells (FIG. 18A) and percent CD8+PD1+ cells (FIG. 18B) after the indicated treatments.

FIG. 19A and FIG. 19B are bar graphs showing percent CD8+ granzyme B+ cells (FIG. 19A) and CD8+:Treg ratio (FIG. 19B) after the indicated treatments.

FIG. 20A and FIG. 20B are bar graphs showing TCR clonality in blood (FIG. 20A) and TCR clonality in tumor (FIG. 20B) after the indicated treatments.

FIG. 21A and FIG. 21B are bar graphs showing TNF-a levels (FIG. 21A) and IFN-g levels (FIG. 21B) after the indicated treatments. Synagis: isotype control for the OX40 antibody.

### DETAILED DESCRIPTION OF THE INVENTION

### Compositions and Combinations

Improved function of the immune system is a goal of immunotherapy for cancer. While not being bound by theory, it is thought that for the immune system to be activated and effectively cause regression or eliminate tumors, there must be efficient cross talk among the various compartments of the immune system as well at the at the tumor bed. The tumoricidal effect is dependent on one or more steps, e.g. the uptake of antigen by immature dendritic cells and presentation of processed antigen via MHC I and II by mature dendritic cells to naïve CD8 (cytotoxic) and CD4 (helper) lymphocytes, respectively, in the draining lymph nodes. Naive T cells express molecules such as CTLA-4 and CD28 that engage with co-stimulatory molecules of the B7 family on antigen presenting cells (APCs) such as dendritic cells. In order to keep T cells in check during immune surveillance, B7 on APCs preferentially binds to CTLA-4, an inhibitory molecule on T lymphocytes. However, upon engagement of the T cell receptor (TCR) with MHC Class I or II receptors via cognate peptide presentation on APCs, the co-stimulatory molecule disengages from CTLA-4 and instead binds to the lower affinity stimulatory molecule CD28, causing T cell activation and proliferation. This expanded population of primed T lymphocytes retains memory of the antigen that was presented to them as they traffic to distant tumor sites. Upon encountering a tumor cell bearing the cognate antigen, they eliminate the tumor via cytolytic mediators such as granzyme B and perforins. This apparently simplistic sequence of events is highly dependent on several cytokines, co-stimulatory molecules and check point modulators to activate and differentiate these primed T lymphocytes to a memory pool of cells that can eliminate the tumor.

Thus, an emerging immunotherapeutic strategy is to target T cell co-stimulatory molecules, e.g. OX40. OX40 (e.g. human OX40 (hOX40) or hOX40R) is a tumor necrosis factor receptor family member that is expressed, among other cells, on activated CD4 and CD8 T cells. One of its functions is in the differentiation and long-term survival of these cells. The ligand for OX40 (OX40L) is expressed by activated antigen-presenting cells. Not wishing to be bound by theory, the anti-OX40 ABPs of a combination of the invention, or a method or use thereof, modulate OX40 and promote growth and/or differentiation of T cells and increase long-term memory T-cell populations, e.g. in overlapping mechanisms as those of OX40L, by “engaging” OX40. Thus, in one embodiment of the ABPs of a combination of the invention, or a method or use thereof, bind and engage OX40. In another embodiment, the anti-OX40 ABPs of a combination of the invention, or a method or use thereof, modulate OX40. In a further embodiment, the ABPs of a combination of the invention, or a method or use thereof, modulate OX40 by mimicking OX40L. In another embodiment, the anti-OX40 ABPs of a combination of the invention, or a method or use thereof, are agonist antibodies. In another embodiment, the anti-OX40 ABPs of a combination of the invention, or a method or use thereof, modulate OX40 and cause proliferation of T cells. In a further embodiment, the anti-OX40 ABPs of a combination of the invention, or a method or use thereof, modulate OX40 and improve, augment, enhance, or increase proliferation of CD4 T cells. In another embodiment, the anti-OX40 ABPs of a combination of the invention, or a method or use thereof, improve, augment, enhance, or increase proliferation of CD8 T cells. In further embodiment, the anti-OX40 ABPs of a combination of the invention, or a method or use thereof, improve, augment, enhance, or increase proliferation of both CD4 and CD8 T cells. In another embodiment, the anti-OX40 ABPs of a combination of the invention, or a method or use thereof, enhance T cell function, e.g. of CD4 or CD8 T cells, or both CD4 and CD8 T cells. In a further embodiment, the anti-OX40 ABPs of a combination of the invention, or a method or use thereof, enhance effector T cell function. In another embodiment, the anti-OX40 ABPs of a combination of the invention, ora method or use thereof, improve, augment, enhance, or increase long-term survival of CD8 T cells. In further embodiments, any of the preceding effects occur in a tumor microenvironment.

Not being bound by theory, of equal importance is the blockade of a potentially robust immunosuppressive response at the tumor site by mediators produced both by T regulatory cells (Tregs) as well as the tumor itself (e.g. Transforming Growth Factor (TGF-B) and interleukin-10 (IL-10)). Not wishing to be bound by theory, a key immune pathogenesis of cancer can be the involvement of Tregs that are found in tumor beds and sites of inflammation. In general, Treg cells occur naturally in circulation and help the immune system to return to a quiet, although vigilant state, after encountering and eliminating external pathogens. They help to maintain tolerance to self antigens and are naturally suppressive in function. They are phenotypically characterized as CD4+, CD25+, FOXP3+ cells. Not wishing to be bound by theory, but in order to break tolerance to effectively treat certain cancers, one mode of therapy is to eliminate Tregs preferentially at tumor sites. Targeting and eliminating Tregs leading to an antitumor response has been more successful in tumors that are immunogenic compared to those that are poorly immunogenic. Many tumors secrete cytokines, e.g. TGF-B that may hamper the immune response by causing precursor CD4+25+ cells to acquire the FOXP3+ phenotype and function as Tregs.

“Modulate” as used herein, for example with regard to a receptor or other target means to change any natural or existing function of the receptor, for example it means affecting binding of natural or artificial ligands to the receptor or target; it includes initiating any partial or full conformational changes or signaling through the receptor or target, and also includes preventing partial or full binding of the receptor or target with its natural or artificial ligands. Also included in the case of membrane bound receptors or targets are any changes in the way the receptor or target interacts with other proteins or molecules in the membrane or change in any localization (or co-localization with other molecules) within membrane compartments as compared to its natural or unchanged state. Modulators are therefore compounds or ligands or molecules that modulate a target or receptor. Modulate includes agonizing, e.g. signaling, as well as antagonizing, or blocking signaling or interactions with a ligand or compound or molecule that happen in the unchanged or unmodulated state. Thus, modulators may be agonists or antagonists. Further, one of skill in the art will recognize that not all modulators will be have absolute selectivity for one target or receptor, but are still considered a modulator for that target or receptor; for example, a modulator may also engage multiple targets.

As used herein the term “agonist” refers to an antigen binding protein including but not limited to an antibody, which upon contact with a co-signalling receptor causes one or more of the following (1) stimulates or activates the receptor, (2) enhances, increases or promotes, induces or prolongs an activity, function or presence of the receptor (3) mimics one or more functions of a natural ligand or molecule that interacts with a target or receptor and includes initiating one or more signaling events through the receptor, mimicking one or more functions of a natural ligand, or initiating one or more partial or full conformational changes that are seen in known functioning or signaling through the receptor and/or (4) enhances, increases, promotes or induces the expression of the receptor. Agonist activity can be measured in vitro by various assays know in the art such as, but not limited to, measurement of cell signalling, cell proliferation, immune cell activation markers, cytokine production. Agonist activity can also be measured in vivo by various assays that measure surrogate end points such as, but not limited to the measurement of T cell proliferation or cytokine production.

As used herein the term “antagonist” refers to an antigen binding protein including but not limited to an antibody, which upon contact with a co-signalling receptor causes one or more of the following (1) attenuates, blocks or inactivates the receptor and/or blocks activation of a receptor by its natural ligand, (2) reduces, decreases or shortens the activity, function or presence of the receptor and/or (3) reduces, descrease, abrogates the expression of the receptor. Antagonist activity can be measured in vitro by various assays know in the art such as, but not limited to, measurement of an increase or decrease in cell signalling, cell proliferation, immune cell activation markers, cytokine production. Antagonist activity can also be measured in vivo by various assays that measure surrogate end points such as, but not limited to the measurement of T cell proliferation or cytokine production.

Thus, in one embodiment, an agonist anti-OX40 ABP inhibits the suppressive effect of Treg cells on other T cells, e.g. within the tumor environment.

Accumulating evidence suggests that the ratio of Tregs to T effector cells in the tumor correlates with anti tumor response. Therefore, in one embodiment, the OX40 ABPs (anti-OX40 ABPs) of a combination of the invention, or a method or use thereof, modulate OX40 to augment T effector number and function and inhibit Treg function.

Enhancing, augmenting, improving, increasing, and otherwise changing the anti-tumor effect of OX40 is an object of a combination of the invention, or a method or use thereof. Described herein are combinations of an anti-OX40 ABP of a combination of the invention, or a method or use thereof, and another compound, such as a CTLA-4 modulator (e.g. anti-CTLA-4 ABP) described herein.

Thus, as used herein the term “combination of the invention” refers to a combination comprising an anti-OX40 ABP, suitably an agonist anti-OX40 ABP, and an anti-CTLA-4 ABP, suitably an antagonist anti-CTLA-4 ABP, each of which may be administered separately or simultaneously as described herein.

As used herein, the terms “cancer,” “neoplasm,” and “tumor,” are used interchangeably and in either the singular or plural form, refer to cells that have undergone a malignant transformation or undergone cellular changes that result in aberrant or unregulated growth or hyperproliferation Such changes or malignant transformations usually make such cells pathological to the host organism, thus precancers or precancerous cells that are or could become pathological and require or could benefit from intervention are also intended to be included. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. In other words, the terms herein include cells, neoplasms, cancers, and tumors of any stage, including what a clinician refers to as precancer, tumors, in situ growths, as well as late stage metastatic growths, Tumors may be hematopoietic tumor, for example, tumors of blood cells or the like, meaning liquid tumors. Specific examples of clinical conditions based on such a tumor include leukemia such as chronic myelocytic leukemia or acute myelocytic leukemia; myeloma such as multiple myeloma; lymphoma and the like.

As used herein the term “agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound or a combination or composition of two or more compounds.

By the term “treating” and derivatives thereof as used herein, is meant therapeutic therapy. In reference to a particular condition, treating means: (1) to ameliorate the condition or one or more of the biological manifestations of the condition; (2) to interfere with (a) one or more points in the biological cascade that leads to or is responsible for the condition or (b) one or more of the biological manifestations of the condition; (3) to alleviate one or more of the symptoms, effects or side effects associated with the condition or one or more of the symptoms, effects or side effects associated with the condition or treatment thereof; (4) to slow the progression of the condition or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, “prevention” is understood to refer to the prophylactic administration of a drug to substantially diminish the likelihood or severity of a condition or biological manifestation thereof, or to delay the onset of such condition or biological manifestation thereof. The skilled artisan will appreciate that “prevention” is not an absolute term. Prophylactic therapy is appropriate, for example, when a subject is considered at high risk for developing cancer, such as when a subject has a strong family history of cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The administration of a therapeutically effective amount of the combinations of the invention (or therapeutically effective amounts of each of the components of the combination) are advantageous over the individual component compounds in that the combinations provide one or more of the following improved properties when compared to the individual administration of a therapeutically effective amount of a component compound: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, or vi) an increase in the bioavailability of one or both of the component compounds.

The invention further provides pharmaceutical compositions, which include one or more of the components herein, and one or more pharmaceutically acceptable carriers, diluents, or excipients. The combination of the invention may comprise two pharmaceutical compositions, one comprising an anti-OX40 ABP of the invention, suitably an agonist anti-OX40 ABP, and the other comprising an anti-CTLA-4 ABP, suitably an antagonist anti-CTLA-4 ABP, each of which may have the same or different carriers, diluents or excipients. The carrier(s), diluent(s) or excipient(s) must be acceptable in the sense of being compatible with the other ingredients of the formulation, capable of pharmaceutical formulation, and not deleterious to the recipient thereof.

The components of the combination of the invention, and pharmaceutical compositions comprising such components may be administered in any order, and in different routes; the components and pharmaceutical compositions comprising the same may be administered simultaneously.

In accordance with another aspect of the invention there is also provided a process for the preparation of a pharmaceutical composition including admixing a component of the combination of the invention and one or more pharmaceutically acceptable carriers, diluents or excipients.

The components of the invention may be administered by any appropriate route. For some components, suitable routes include oral, rectal, nasal, topical (including buccal and sublingual), vaginal, and parenteral (including subcutaneous, intramuscular, intraveneous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that the components may be compounded together or in separate pharmaceutical compositions.

In one embodiment, one or more components of a combination of the invention are administered intravenously. In another embodiment, one or more components of a combination of the invention are administered intratumorally. In another embodiment, one or more components of a combination of the invention are administered systemically, e.g. intravenously, and one or more other components of a combination of the invention are administered intratumorally. In another embodiment, all of the components of a combination of the invention are administered systemically, e.g. intravenously. In an alternative embodiment, all of the components of the combination of the invention are administered intratumorally. In any of the embodiments, e.g. in this paragraph, the components of the invention are administered as one or more pharmaceutical compositions.

Antigen Binding Proteins that Bind OX40

“Antigen Binding Protein (ABP)” means a protein that binds an antigen, including antibodies or engineered molecules that function in similar ways to antibodies. Such alternative antibody formats include triabody, tetrabody, miniantibody, and a minibody, Also included are alternative scaffolds in which the one or more CDRs of any molecules in accordance with the disclosure can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain. An ABP also includes antigen binding fragments of such antibodies or other molecules. Further, an ABP of a combination of the invention, or a method or use thereof, may comprise the VH regions formatted into a full length antibody, a (Fab′)2 fragment, a Fab fragment, a bi-specific or biparatopic molecule or equivalent thereof (such as scFV, bi- tri- or tetra-bodies, Tandabs etc.), when paired with an appropriate light chain. The ABP may comprise an antibody that is an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. The ABP may also be a chimeric antibody of the type described in WO86/01533 which comprises an antigen binding region and a non-immunoglobulin region.

Thus, herein an anti-OX40 ABP of a combination, or a method or use thereof, of the invention or protein is one that binds OX40, and in preferred embodiments does one or more of the following: modulate signaling through OX40, modulates the function of OX40, agonize OX40 signalling, stimulate OX40 function, or co-stimulate OX40 signaling. One of skill in the art would readily recognize a variety of well known assays to establish such functions.

The term “antibody” as used herein refers to molecules with an antigen binding domain, and optionally an immunoglobulin-like domain or fragment thereof and includes monoclonal (for example IgG, IgM, IgA, IgD or IgE and modified variants thereof), recombinant, polyclonal, chimeric, humanized, biparatopic, bispecific and heteroconjugate antibodies, or a closed conformation multispecific antibody. An “antibody” included xenogeneic, allogeneic, syngeneic, or other modified forms thereof. An antibody may be isolated or purified. An antibody may also be recombinant, i.e. produced by recombinant means; for example, an antibody that is 90% identical to a reference antibody may be generated by mutagenesis of certain residues using recombinant molecular biology techniques known in the art. Thus, the antibodies of the present invention may comprise heavy chain variable regions and light chain variable regions of a combination of the invention, or a method or use thereof, which may be formatted into the structure of a natural antibody or formatted into a full length recombinant antibody, a (Fab′)2 fragment, a Fab fragment, a bi-specific or biparatopic molecule or equivalent thereof (such as scFV, bi- tri- or tetra-bodies, Tandabs etc.), when paired with an appropriate light chain. The antibody may be an IgG1, IgG2, IgG3, or IgG4 or a modified variant thereof. The constant domain of the antibody heavy chain may be selected accordingly. The light chain constant domain may be a kappa or lambda constant domain. The antibody may also be a chimeric antibody of the type described in WO86/01533 which comprises an antigen binding region and a non-immunoglobulin region.

One of skill in the art will recognize that the anti-OX40 ABPs of a combination herein, or method or use therof, of the invention bind an epitope of OX40; likewise an anti-CTLA-4 ABP of a combination herein, or a method or use thereof, of the invention binds an epitope of CTLA-4. The epitope of an ABP is the region of its antigen to which the ABP binds. Two ABPs bind to the same or overlapping epitope if each competitively inhibits (blocks) binding of the other to the antigen. That is, a 1×, 5×, 10×, 20× or 100× excess of one antibody inhibits binding of the other by at least 50% but preferably 75%, 90% or even 99% as measured in a competitive binding assay compared to a control lacking the competing antibody (see, e.g., Junghans et al., Cancer Res. 50:1495, 1990, which is incorporated herein by reference). Alternatively, two antibodies have the same epitope if essentially all amino acid mutations in the antigen that reduce or eliminate binding of one antibody reduce or eliminate binding of the other. Also the same epitope may include “overlapping epitopes” e.g. if some amino acid mutations that reduce or eliminate binding of one antibody reduce or eliminate binding of the other.

The strength of binding may be important in dosing and administration of an ABP of the combination, or method or use thereof, of the invention. In one embodiment, the ABP of the invention binds its target (e.g. OX40 or CTLA-4) with high affinity. For example, when measured by Biacore, the antibody binds to OX40, preferably human OX40, with an affinity of 1-1000 nM or 500 nM or less or an affinity of 200 nM or less or an affinity of 100 nM or less or an affinity of 50 nM or less or an affinity of 500 pM or less or an affinity of 400 pM or less, or 300 pM or less. In a further aspect the antibody binds to OX40, preferably human OX40, when measured by Biacore of between about 50 nM and about 200 nM or between about 50 nM and about 150 nM. In one aspect of the present invention the antibody binds OX40, preferably human OX40, with an affinity of less than 100 nM.

In a further embodiment, binding is measured by Biacore. Affinity is the strength of binding of one molecule, e.g. an antibody of a combination of the invention, or a method or use thereof, to another, e.g. its target antigen, at a single binding site. The binding affinity of an antibody to its target may be determined by equilibrium methods (e.g. enzyme-linked immunoabsorbent assay (ELISA) or radioimmunoassay (RIA)), or kinetics (e.g. BIACORE analysis). For example, the Biacore methods known in the art may be used to measure binding affinity.

Avidity is the sum total of the strength of binding of two molecules to one another at multiple sites, e.g. taking into account the valency of the interaction.

In an aspect, the equilibrium dissociation constant (KD) of the ABP of a combination of the invention, or a method or use thereof, and OX40, preferably human OX40, interaction is 100 nM or less, 10 nM or less, 2 nM or less or 1 nM or less. Alternatively the KD may be between 5 and 10 nM; or between 1 and 2 nM. The KD may be between 1 pM and 500 pM; or between 500 pM and 1 nM. A skilled person will appreciate that the smaller the KD numerical value, the stronger the binding. The reciprocal of KD (i.e. 1/KD) is the equilibrium association constant (KA) having units M-1. A skilled person will appreciate that the larger the KA numerical value, the stronger the binding.

The dissociation rate constant (kd) or “off-rate” describes the stability of the complex of the ABP on one hand and OX40, preferably human OX40 on the other hand, i.e. the fraction of complexes that decay per second. For example, a kd of 0.01 s-1 equates to 1% of the complexes decaying per second. In an embodiment, the dissociation rate constant (kd) is 1×10-3 s-1 or less, 1×10-4 s-1 or less, 1×10-5 s-1 or less, or 1×10-6 s-1 or less. The kd may be between 1×10-5 s-1 and 1×10-4 s-1; or between 1×10-4 s-1 and 1×10-3 s-1.

Competition between an anti-OX40 ABP of a combination of the invention, or a method or use thereof, and a reference antibody, e.g. for binding OX40, an epitope of OX40, or a fragment of the OX40, may be determined by competition ELISA, FMAT or Biacore. In one aspect, the competition assay is carried out by Biacore. There are several possible reasons for this competition: the two proteins may bind to the same or overlapping epitopes, there may be steric inhibition of binding, or binding of the first protein may induce a conformational change in the antigen that prevents or reduces binding of the second protein.

“Binding fragments” as used herein means a portion or fragment of the ABPs of a combination of the invention, or a method or use thereof, that include the antigen-binding site and are capable of binding OX40 as defined herein, e.g. but not limited to capable of binding to the same epitope of the parent or full length antibody.

Functional fragments of the ABPs of a combination of the invention, or a method or use thereof, are contemplated herein.

Thus, “binding fragments” and “functional fragments” may be an Fab and F(ab′)2 fragments which lack the Fc fragment of an intact antibody, clear more rapidly from the circulation, and may have less non-specific tissue binding than an intact antibody (Wahl et al., J. Nuc. Med. 24:316-325 (1983)). Also included are Fv fragments (Hochman, J. et al. (1973) Biochemistry 12:1130-1135; Sharon, J. et al. (1976) Biochemistry 15:1591-1594). These various fragments are produced using conventional techniques such as protease cleavage or chemical cleavage (see, e.g., Rousseaux et al., Meth. Enzymol., 121:663-69 (1986)).

“Functional fragments” as used herein means a portion or fragment of the ABPs of a combination of the invention, or a method or use thereof, that include the antigen-binding site and are capable of binding the same target as the parent ABP, e.g. but not limited to binding the same epitope, and that also retain one or more modulating or other functions described herein or known in the art.

As the ABPs of the present invention may comprise heavy chain variable regions and light chain variable regions of a combination of the invention, or a method or use thereof, which may be formatted into the structure of a natural antibody, a functional fragment is one that retains binding or one or more functions of the full length ABP as described herein. A binding fragment of an ABP of a combination of the invention, or a method or use thereof, may therefore comprise the VL or VH regions, a (Fab′)2 fragment, a Fab fragment, a fragment of a bi-specific or biparatopic molecule or equivalent thereof (such as scFV, bi- tri- or tetra-bodies, Tandabs etc.), when paired with an appropriate light chain.

The term “CDR” as used herein, refers to the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin.

It will be apparent to those skilled in the art that there are various numbering conventions for CDR sequences; Chothia (Chothia et al. (1989) Nature 342: 877-883), Kabat (Kabat et al., Sequences of Proteins of Immunological Interest, 4th Ed., U.S. Department of Health and Human Services, National Institutes of Health (1987)), AbM (University of Bath) and Contact (University College London). The minimum overlapping region using at least two of the Kabat, Chothia, AbM and contact methods can be determined to provide the “minimum binding unit”. The minimum binding unit may be a subportion of a CDR. The structure and protein folding of the antibody may mean that other residues are considered part of the CDR sequence and would be understood to be so by a skilled person. It is noted that some of the CDR definitions may vary depending on the individual publication used.

Unless otherwise stated and/or in absence of a specifically identified sequence, references herein to “CDR”, “CDRL1” (or “LC CDR1”), “CDRL2” (or “LC CDR2”), “CDRL3” (or “LC CDR3”), “CDRH1” (or “HC CDR1”), “CDRH2” (or “HC CDR2”), “CDRH3” (or “HC CDR3”) refer to amino acid sequences numbered according to any of the known conventions; alternatively, the CDRs are referred to as “CDR1,” “CDR2,” “CDR3” of the variable light chain and “CDR1,” “CDR2,” and “CDR3” of the variable heavy chain. In particular embodiments, the numbering convention is the Kabat convention.

The term “CDR variant” as used herein, refers to a CDR that has been modified by at least one, for example 1, 2 or 3, amino acid substitution(s), deletion(s) or addition(s), wherein the modified antigen binding protein comprising the CDR variant substantially retains the biological characteristics of the antigen binding protein pre-modification. It will be appreciated that each CDR that can be modified may be modified alone or in combination with another CDR. In one aspect, the modification is a substitution, particularly a conservative substitution, for example as shown in Table 1.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

For example, in a variant CDR, the amino acid residues of the minimum binding unit may remain the same, but the flanking residues that comprise the CDR as part of the Kabat or Chothia definition(s) may be substituted with a conservative amino acid residue.

Such antigen binding proteins comprising modified CDRs or minimum binding units as described above may be referred to herein as “functional CDR variants” or “functional binding unit variants”.

The antibody may be of any species, or modified to be suitable to administer to a cross species. For example the CDRs from a mouse antibody may be humanized for administration to humans. In any embodiment, the antigen binding protein is optionally a humanized antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., Queen et al., Proc. Natl Acad Sci USA, 86:10029-10032 (1989), Hodgson et al., Bio/Technology, 9:421 (1991)). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanised antibodies—see for example EP-A-0239400 and EP-A-054951.

In yet a further embodiment, the humanized antibody has a human antibody constant region that is an IgG. In another embodiment, the IgG is a sequence as disclosed in any of the above references or patent publications.

For nucleotide and amino acid sequences, the term “identical” or “identity” indicates the degree of identity between two nucleic acid or two amino acid sequences when optimally aligned and compared with appropriate insertions or deletions.

The percent sequence identity between two sequences is a function of the number of identical positions shared by the sequences (i.e., % identity=number of identical positions/total number of positions multiplied by 100), taking into account the number of gaps, and the length of each gap, which need to be introduced for optimal alignment of the two sequences. The comparison of sequences and determination of percent identity between two sequences can be accomplished using a mathematical algorithm, as described below.

Percent identity between a query nucleic acid sequence and a subject nucleic acid sequence is the “Identities” value, expressed as a percentage, which is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein.

Percent identity between a query amino acid sequence and a subject amino acid sequence is the “Identities” value, expressed as a percentage, which is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

In any embodiment of a combination of the invention, or a method or use thereof, herein, the ABP may have any one or all CDRs, VH, VL, HC, LC, with 99, 98, 97, 96, 95, 94, 93, 92, 91, or 90, or 85, or 80, or 75, or 70 percent identity to the sequence shown or referenced, e.g. as defined by a SEQ ID NO disclosed herein.

ABPs that bind human OX40 receptor are provided herein (i.e. an anti-OX40 ABP and an anti-human OX40 receptor (hOX-40R) antibody, sometimes referred to herein as an “anti-OX40 ABP” or an “anti-OX40 antibody” and/or other variations of the same). These antibodies are useful in the treatment or prevention of acute or chronic diseases or conditions whose pathology involves OX40 signalling. In one aspect, an antigen binding protein, or isolated human antibody or functional fragment of such protein or antibody, that binds to human OX40R and is effective as a cancer treatment or treatment against disease is described, for example in combination with another compound such as an anti-CTLA-4 ABP, suitably an antagonist anti-CTLA-4 ABP. Any of the antigen binding proteins or antibodies disclosed herein may be used as a medicament. Any one or more of the antigen binding proteins or antibodies may be used in the methods or compositions to treat cancer, e.g. those disclosed herein.

The isolated antibodies as described herein bind to OX40, and may bind to OX40 encoded from the following genes: NCBI Accession Number NP_003317, Genpept Accession Number P23510, or genes having 90 percent homology or 90 percent identity thereto. The isolated antibody provided herein may further bind to the OX40 receptor having one of the following GenBank Accession Numbers: AAB39944, CAE11757, or AAI05071.

Antigen binding proteins and antibodies that bind and/or modulate OX-40 receptor are known in the art. Exemplary anti-OX40 ABPs of a combination of the invention, or a method or use thereof, are disclosed, for example in International Publication No. WO2013/028231 (PCT/US2012/024570), international filing date 9 Feb. 2012, and WO2012/027328 (PCT/US2011/048752), international filing date 23 Aug. 2011, each of which is incorporated by reference in its entirety herein (To the extent any definitions conflict, this instant application controls).

In one embodiment, the OX-40 antigen binding protein is one disclosed in WO2012/027328 (PCT/US2011/048752), international filing date 23 Aug. 2011. In another embodiment, the antigen binding protein comprises the CDRs of an antibody disclosed in WO2012/027328 (PCT/US2011/048752), international filing date 23 Aug. 2011, or CDRs with at least 90% (e.g., 90%, 91%, 92%, 93%, 94%, 95%, 96%, 97%, 98%, or 99%, 100%) sequence identity to the disclosed CDR sequences. In a further embodiment the antigen binding protein comprises a VH, a VL, or both of an antibody disclosed in WO2012/027328 (PCT/US2011/048752), international filing date 23 Aug. 2011, or a VH or a VL with at least 90% (e.g., 90%, 91%, 92%, 93%, 94%, 95%, 96%, 97%, 98%, or 99%, 100%) sequence identity to the disclosed VH or VL sequences.

In another embodiment, the OX-40 antigen binding protein is one disclosed in WO2013/028231 (PCT/US2012/024570), international filing date 9 Feb. 2012. In another embodiment, the antigen binding protein comprises the CDRs of an antibody disclosed in WO2013/028231 (PCT/US2012/024570), international filing date 9 Feb. 2012, or CDRs with at least 90% (e.g., 90%, 91%, 92%, 93%, 94%, 95%, 96%, 97%, 98%, or 99%, 100%) sequence identity to the disclosed CDR sequences. In a further embodiment the antigen binding protein comprises a VH, a VL, or both of an antibody disclosed in WO2013/028231 (PCT/US2012/024570), international filing date 9 Feb. 2012, or a VH or a VL with at least 90% (e.g., 90%, 91%, 92%, 93%, 94%, 95%, 96%, 97%, 98%, or 99%, 100%) sequence identity to the disclosed VH or VL sequences.

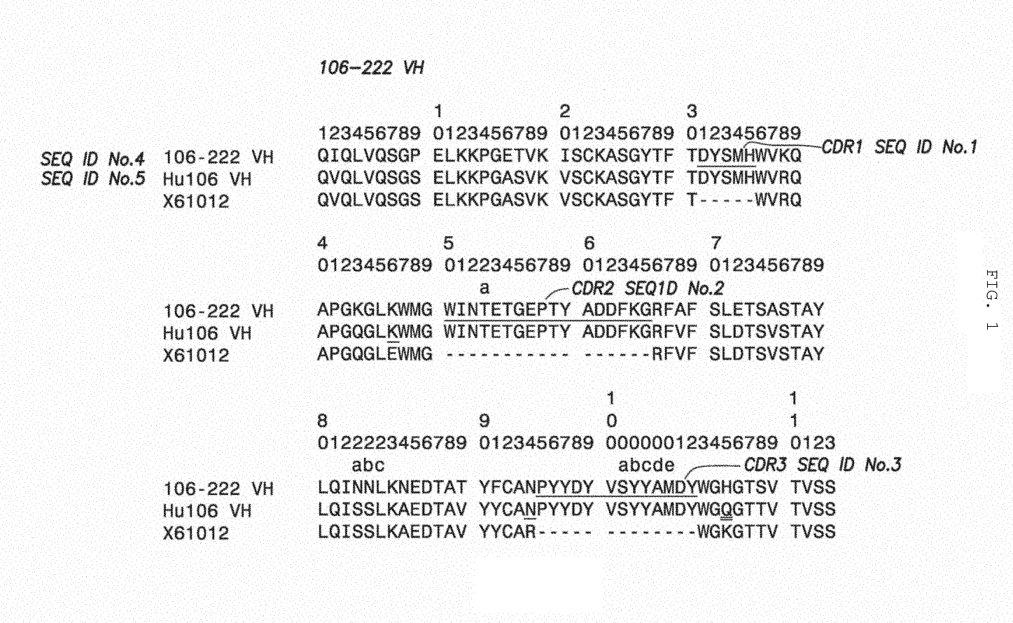

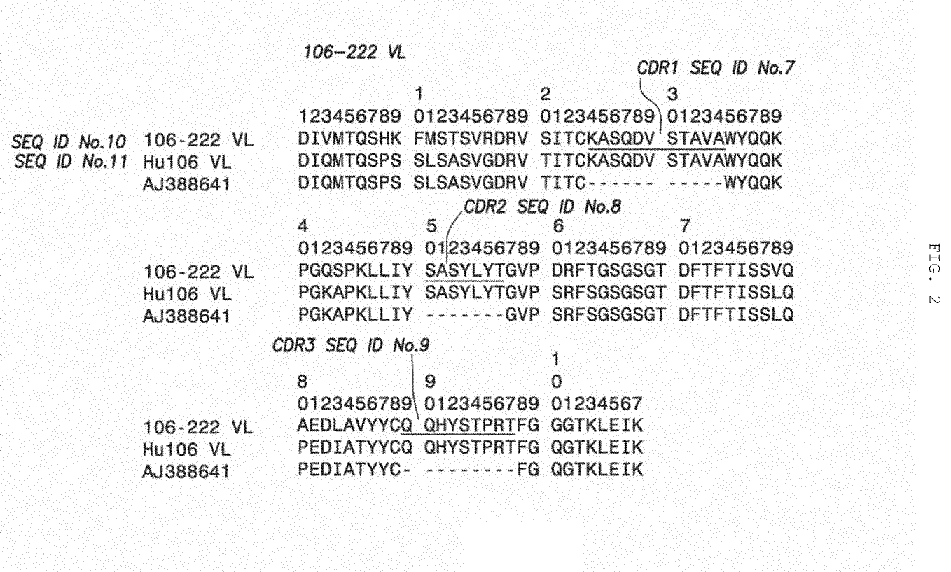

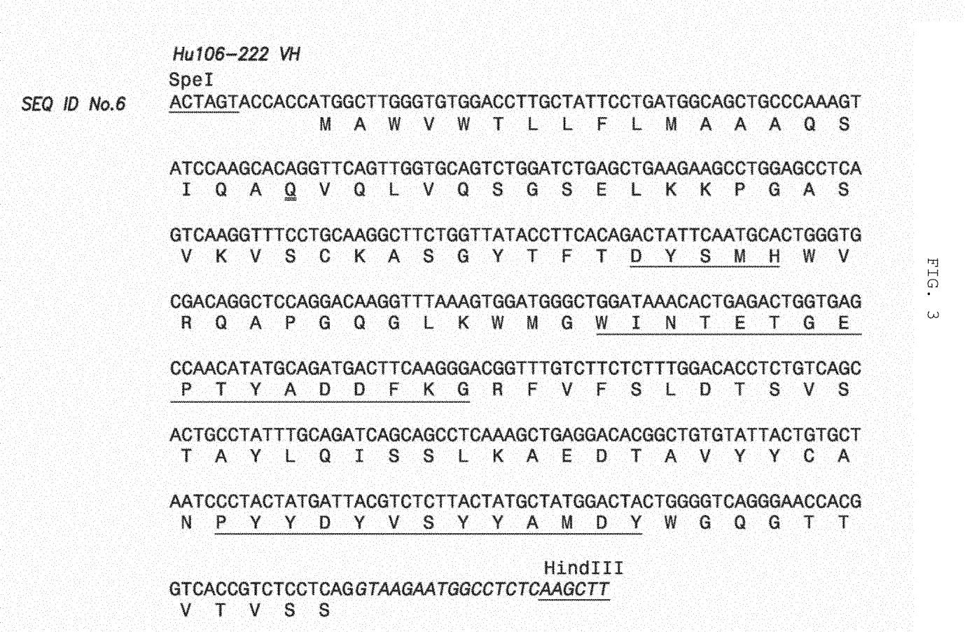

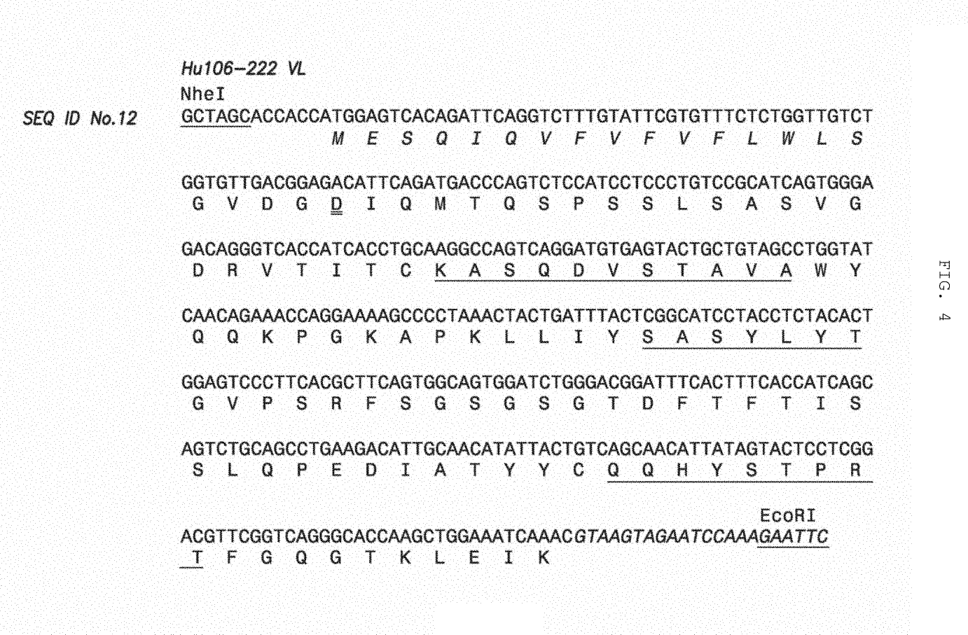

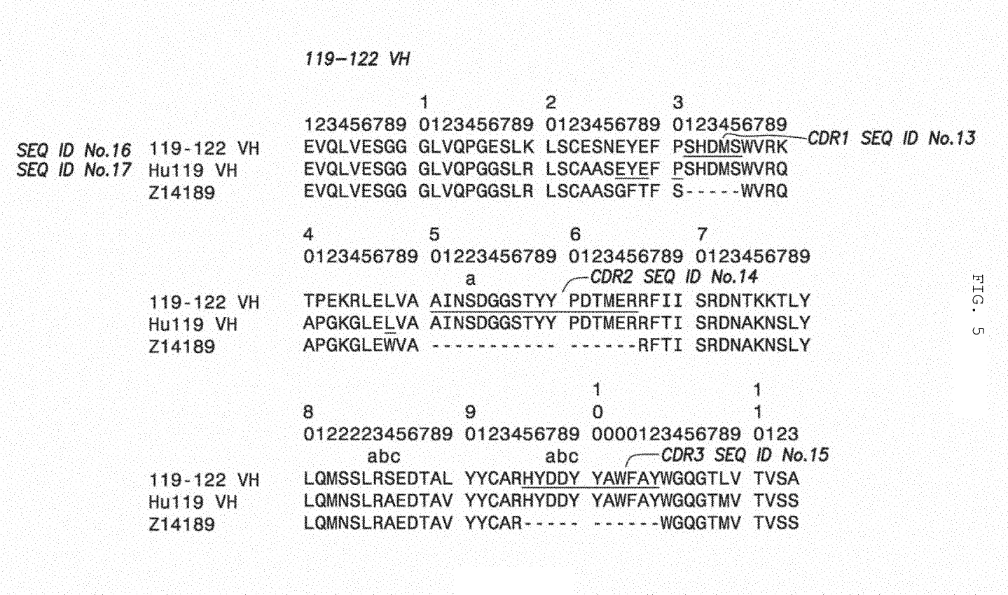

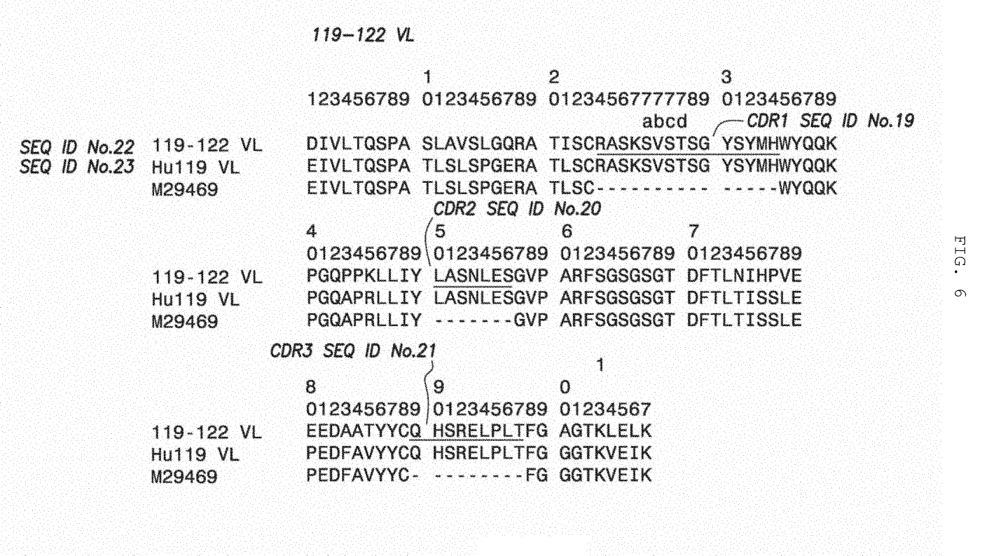

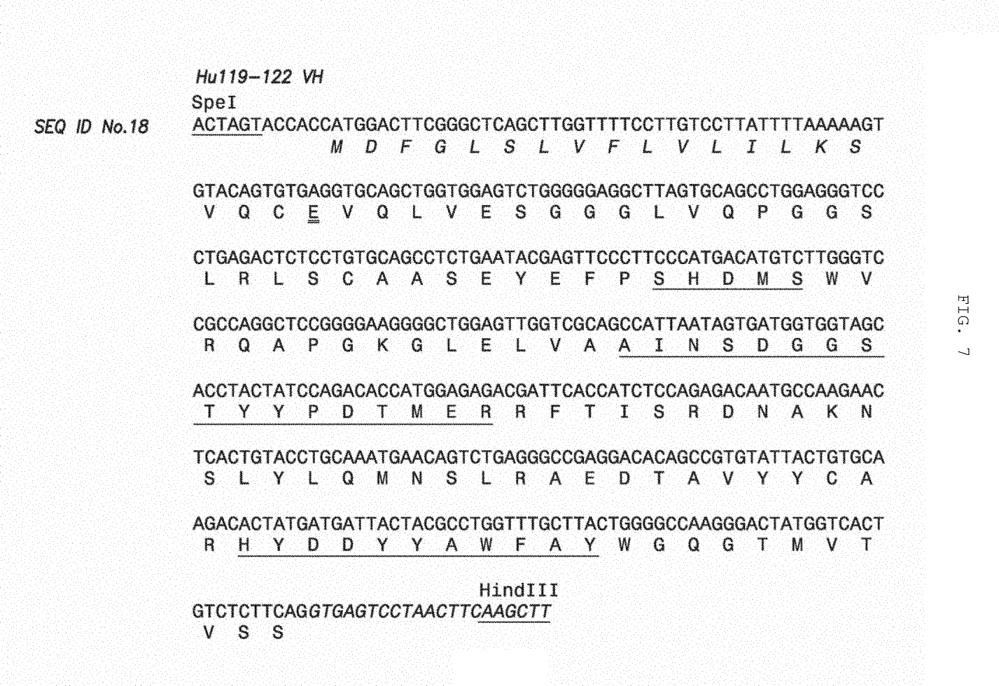

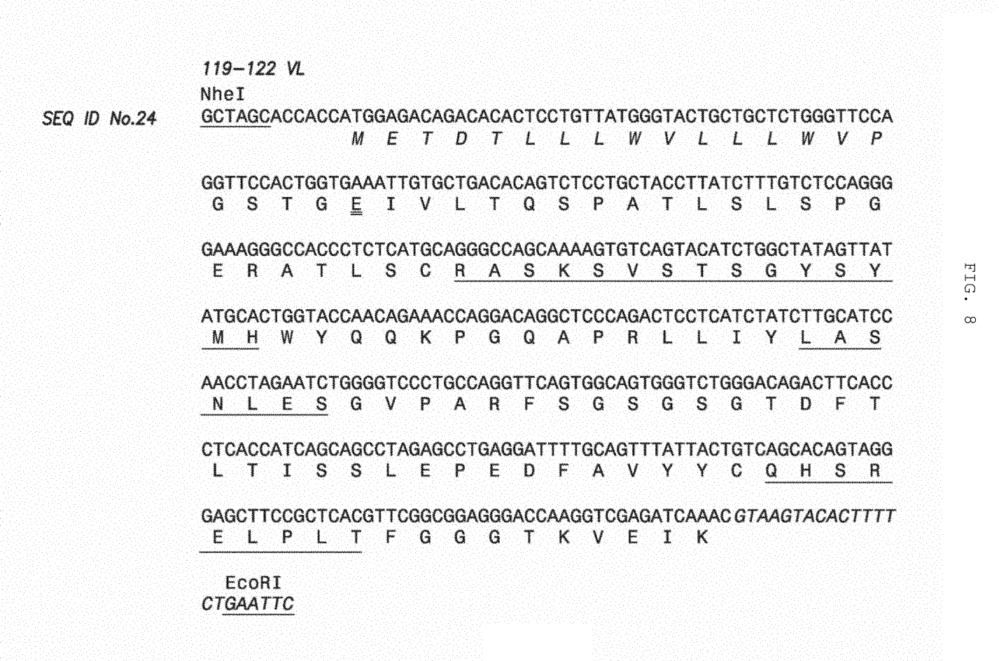

FIGS. 1-12 show sequences of the anti-OX40 ABPs of a combination of the invention, or a method or use thereof, e.g. CDRs and VH and VL sequences of the ABPs. In another embodiment, the anti-OX40 ABP of a combination of the invention, or a method or use thereof, comprises one or more of the CDRs or VH or VL sequences, or sequences with at least 90% (e.g., 90%, 91%, 92%, 93%, 94%, 95%, 96%, 97%, 98%, or 99%, 100%) sequence identity thereto, shown in the Figures herein. FIG. 1 includes a disclosure of residues 1-30, 36-49, 67-98, and 121-131 of SEQ ID NO: 70. X61012 is disclosed as SEQ ID NO: 70. FIG. 2 includes a disclosure of residues 1-23, 35-49, 57-88, and 102-111 of SEQ ID NO: 71. AJ388641 is disclosed as SEQ ID NO: 71. FIG. 3 includes a disclosure of the amino acid sequence as SEQ ID NO: 72. FIG. 4 includes a disclosure of the amino acid sequence as SEQ ID NO: 73. FIG. 5 includes a disclosure of residues 17-46, 52-65, 83-114, and 126-136 of SEQ ID NO: 74. Z14189 is disclosed as SEQ ID NO: 74. FIG. 6 includes a disclosure of residues 21-43, 55-69, 77-108, and 118-127 of SEQ ID NO: 75. M29469 is disclosed as SEQ ID NO: 75. FIG. 7 includes a disclosure of the amino acid sequence as SEQ ID NO: 76. FIG. 8 includes a disclosure of the amino acid sequence as SEQ ID NO: 77.

FIG. 1 shows the alignment of the amino acid sequences of 106-222, humanized 106-222 (Hu106), and human acceptor X61012 (GenBank accession number) VH sequences are shown. Amino acid residues are shown in single letter code. Numbers above the sequences indicate the locations according to Kabat et al. (Sequences of Proteins of Immunological Interests, Fifth edition, NIH Publication No. 91-3242, U.S. Department of Health and Human Services, 1991). The same sequences as claimed herein are also provided in the Sequence Listing and the position numbers may be different. In FIG. 1, CDR sequences defined by Kabat et al. (1991) are underlined in 106-222 VH. CDR residues in X61012 VH are omitted in the figure. Human VH sequences homologous to the 106-222 VH frameworks were searched for within the GenBank database, and the VH sequence encoded by the human X61012 cDNA (X61012 VH) was chosen as an acceptor for humanization. The CDR sequences of 106-222 VH were first transferred to the corresponding positions of X61012 VH. Next, at framework positions where the three-dimensional model of the 106-222 variable regions suggested significant contact with the CDRs, amino acid residues of mouse 106-222 VH were substituted for the corresponding human residues. These substitutions were performed at positions 46 and 94 (underlined in Hu106 VH). In addition, a human framework residue that was found to be atypical in the corresponding V region subgroup was substituted with the most typical residue to reduce potential immunogenicity. This substitution was performed at position 105 (double-underlined in Hu106 VH).

FIG. 2 shows alignment of the amino acid sequences of 106-222, humanized 106-222 (Hu106), and human acceptor AJ388641 (GenBank accession number) VL sequences is shown. Amino acid residues are shown in single letter code. Numbers above the sequences indicate the locations according to Kabat et al. (1991). The same sequences as claimed herein are also provided in the Sequence Listing although the position numbers may be different. CDR sequences defined by Kabat et al. are underlined in 106-222 VH. CDR residues in AJ388641 VL are omitted in the figure. Human VL sequences homologous to the 106-222 VL frameworks were searched for within the GenBank database, and the VL sequence encoded by the human AJ388641 cDNA (AJ388641 VL) was chosen as an acceptor for humanization. The CDR sequences of 106-222 VL were transferred to the corresponding positions of AJ388641 VL. No framework substitutions were performed in the humanized form.

FIG. 3 shows the nucleotide sequence of the Hu106 VH gene flanked by SpeI and HindIII sites (underlined) is shown along with the deduced amino acid sequence. Amino acid residues are shown in single letter code. The signal peptide sequence is in italic. The N-terminal amino acid residue (Q) of the mature VH is double-underlined. CDR sequences according to the definition of Kabat et al. (1991) are underlined. The same sequences as claimed herein are also provided in the Sequence Listing and the position numbers may be different in the Sequence Listing. The intron sequence is in italic.

FIG. 4 shows the nucleotide sequence of the Hu106-222 VL gene flanked by NheI and EcoRI sites (underlined) is shown along with the deduced amino acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined. The intron sequence is in italic. The same sequences as claimed herein are also provided in the Sequence Listing although the position numbers may be different in the Sequence Listing.

FIG. 5 shows the alignment of the amino acid sequences of 119-122, humanized 119-122 (Hu119), and human acceptor Z14189 (GenBank accession number) VH sequences are shown. Amino acid residues are shown in single letter code. Numbers above the sequences indicate the locations according to Kabat et al. (Sequences of Proteins of Immunological Interests, Fifth edition, NIH Publication No. 91-3242, U.S. Department of Health and Human Services, 1991). CDR sequences defined by Kabat et al. (1991) are underlined in 119-122 VH. CDR residues in Z14189 VH are omitted in the figure. Human VH sequences homologous to the 119-122 VH frameworks were searched for within the GenBank database, and the VH sequence encoded by the human Z14189 cDNA (Z14189 VH) was chosen as an acceptor for humanization. The CDR sequences of 119-122 VH were first transferred to the corresponding positions of Z14189 VH. Next, at framework positions where the three-dimensional model of the 119-122 variable regions suggested significant contact with the CDRs, amino acid residues of mouse 119-122 VH were substituted for the corresponding human residues. These substitutions were performed at positions 26, 27, 28, 30 and 47 (underlined in the Hu119 VH sequence) as shown on the figure. The same sequences as claimed herein are also provided in the Sequence Listing although the position numbers may be different in the Sequence Listing.

FIG. 6 shows the alignment of the amino acid sequences of 119-122, humanized 119-122 (Hu119), and human acceptor M29469 (GenBank accession number) VL sequences are shown. Amino acid residues are shown in single letter code. Numbers above the sequences indicate the locations according to Kabat et al. (1991). CDR sequences defined by Kabat et al. (1) are underlined in 119-122 VL. CDR residues in M29469 VL are omitted in the sequence. Human VL sequences homologous to the 119-122 VL frameworks were searched for within the GenBank database, and the VL sequence encoded by the human M29469 cDNA (M29469 VL) was chosen as an acceptor for humanization. The CDR sequences of 119-122 VL were transferred to the corresponding positions of M29469 VL.

No framework substitutions were needed in the humanized form. The same sequences as claimed herein are also provided in the Sequence Listing although the position numbers may be different in the Sequence Listing.

FIG. 7 shows the nucleotide sequence of the Hu119 VH gene flanked by SpeI and HindIII sites (underlined) is shown along with the deduced amino acid sequence. Amino acid residues are shown in single letter code. The signal peptide sequence is in italic. The N-terminal amino acid residue (E) of the mature VH is double-underlined. CDR sequences according to the definition of Kabat et al. (1991) are underlined. The intron sequence is in italic. The same sequences as claimed herein are also provided in the Sequence Listing although the position numbers may be different in the Sequence Listing.

FIG. 8 shows the nucleotide sequence of the Hu119 VL gene flanked by NheI and EcoRI sites (underlined) is shown along with the deduced amino acid sequence. Amino acid residues are shown in single letter code. The signal peptide sequence is in italic. The N-terminal amino acid residue (E) of the mature VL is double-underlined. CDR sequences according to the definition of Kabat et al. (1991) are underlined. The intron sequence is in italic. The same sequences as claimed herein are also provided in the Sequence Listing although the position numbers may be different in the Sequence Listing.

FIG. 9 shows the nucleotide sequence of mouse 119-43-1 VH cDNA along with the deduced amino acid sequence. Amino acid residues are shown in single letter code. The signal peptide sequence is in italic. The N-terminal amino acid residue (E) of the mature VH is double-underlined. CDR sequences according to the definition of Kabat et al. (Sequences of Proteins of Immunological Interests, Fifth edition, NIH Publication No. 91-3242, U.S. Department of Health and Human Services, 1991) are underlined.

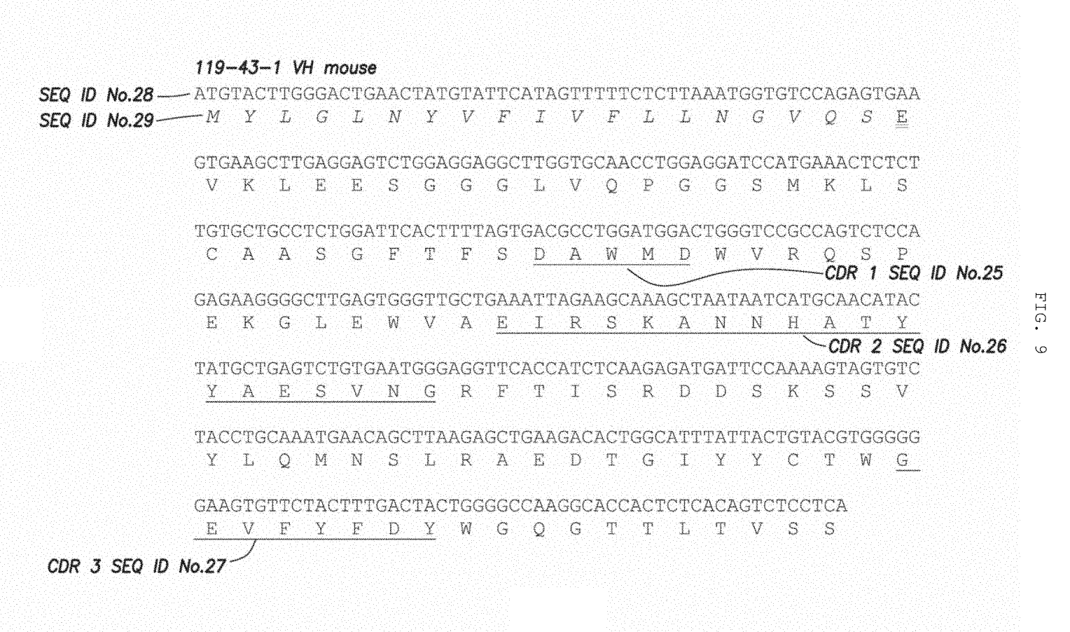

FIG. 10 shows the nucleotide sequence of mouse 119-43-1 VL cDNA is shown the deduced amnno acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined.

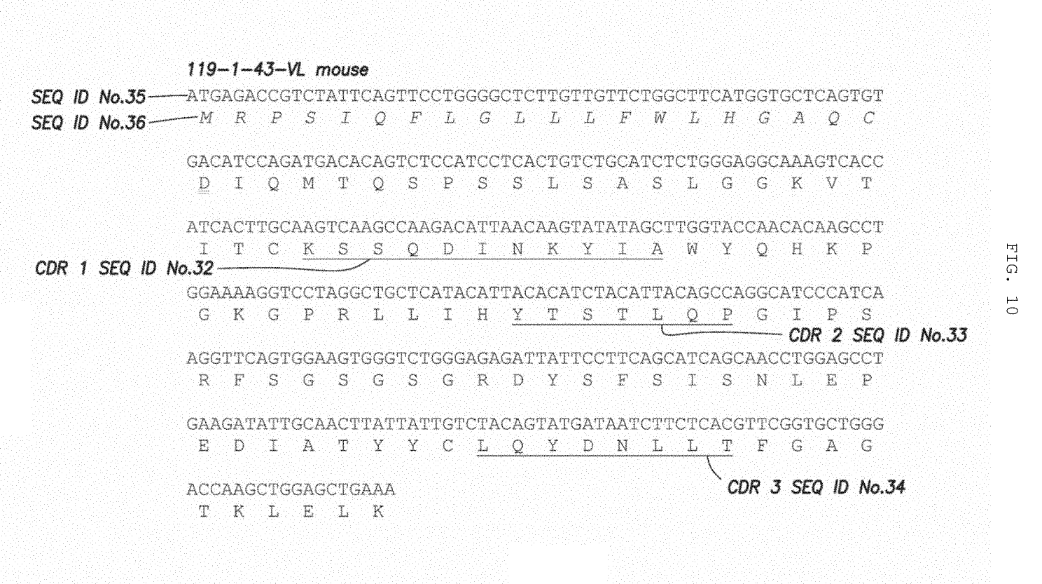

FIG. 11 shows the nucleotide sequence of the designed 119-43-1 VH gene flanked by SpeI and HindIII sites (underlined) along with the deduced amino acid sequence. Amino acid residues are shown in single letter code. The signal peptide sequence is in italic. The N-terminal amino acid residue (E) of the mature VH is double-underlined. CDR sequences according to the definition of Kabat et al. (1991) are underlined. The intron sequence is in italic.

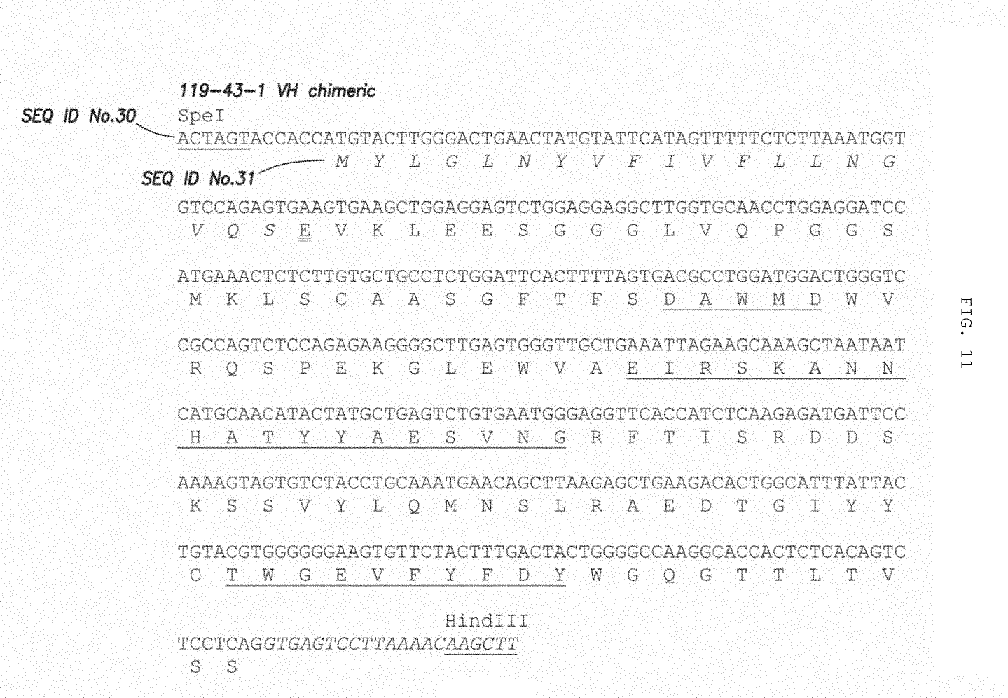

FIG. 12 shows the nucleotide sequence of the designed 119-43-1 VL gene flanked by NheI and EcoRI sites (underlined) along with the deduced amino acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined. The intron sequence is in italic.

In one embodiment, the anti-OX40 ABP of a combination of the invention, or a method or use thereof, comprises the CDRs of the 106-222 antibody, e.g. CDRH1, CDRH2, and CDRH3 having the amino acid sequence as set forth in SEQ ID NOs 1, 2, and 3, and e.g. CDRL1, CDRL2, and CDRL3 having the sequences as set forth in SEQ ID NOs 7, 8, and 9 respectively. In one embodiment, the ABP of a combination of the invention, or a method or use thereof, comprises the CDRs of the 106-222, Hu106 or Hu106-222 antibody as disclosed in WO2012/027328 (PCT/US2011/048752), international filing date 23 Aug. 2011.

As described herein, ANTIBODY 106-222 is a humanized monoclonal antibody that binds to human OX40 as disclosed in WO2012/027328 and described herein an antibody comprising CDRH1, CDRH2, and CDRH3 having the amino acid sequence as set forth in SEQ ID NOs 1, 2, and 3, and e.g. CDRL1, CDRL2, and CDRL3 having the sequences as set forth in SEQ ID NOs 7, 8, and 9, respectively and an antibody comprising VH having an amino acid sequence as set forth in SEQ ID NO:4 and a VL having an amino acid sequence as set forth in SEQ ID NO: 10.

In a further embodiment, the anti-OX40 ABP of a combination of the invention, or a method or use thereof, comprises the VH and VL regions of the 106-222 antibody as shown in FIG. 6 and FIG. 7 herein, e.g. a VH having an amino acid sequence as set forth in SEQ ID NO:4 and a VL having an amino acid sequence as set forth in SEQ ID NO: 10. In another embodiment, the ABP of a combination of the invention, or a method or use thereof, comprises a VH having an amino acid sequence as set forth in SEQ ID NO: 5, and a VL having an amino acid sequence as set forth in SEQ ID NO:11. In a further embodiment, the anti-OX40 ABP of a combination of the invention, or a method or use thereof, comprises the VH and VL regions of the Hu106-222 antibody or the 106-222 antibody or the Hu106 antibody as disclosed in WO2012/027328 (PCT/US2011/048752), international filing date 23 Aug. 2011. In a further embodiment, the anti-OX40 ABP of a combination of the invention, or a method or use thereof, is 106-222, Hu106-222 or Hu106, e.g. as disclosed in WO2012/027328 (PCT/US2011/048752), international filing date 23 Aug. 2011. In a further embodiment, the ABP of a combination of the invention, or a method or use thereof, comprises CDRs or VH or VL or antibody sequences with at least 90% (e.g., 90%, 91%, 92%, 93%, 94%, 95%, 96%, 97%, 98%, or 99%, 100%) sequence identity to the sequences in this paragraph.

In another embodiment, the anti-OX40 ABP of a combination of the invention, or a method or use thereof, comprises the CDRs of the 119-122 antibody, e.g. CDRH1, CDRH2, and CDRH3 having the amino acid sequence as set forth in SEQ ID NOs 13, 14, and 15 respectively. In another embodiment, the anti-OX40 ABP of a combination of the invention, or a method or use thereof, comprises the CDRs of the 119-122 or Hu119 or Hu119-222 antibody as disclosed in WO2012/027328 (PCT/US2011/048752), international filing date 23 Aug. 2011. In a further embodiment, the anti-OX40 ABP of a combination of the invention, or a method or use thereof, comprises a VH having an amino acid sequence as set forth in SEQ ID NO: 16, and a VL having the amino acid sequence as set forth in SEQ ID NO: 22. In another embodiment, the anti-OX40 ABP of a combination of the invention, or a method or use thereof, comprises a VH having an amino acid sequence as set forth in SEQ ID NO: 17 and a VL having the amino acid sequence as set forth in SEQ ID NO: 23. In a further embodiment, the anti-OX40 ABP of a combination of the invention, or a method or use thereof, comprises the VH and VL regions of the 119-122 or Hu119 or Hu119-222 antibody as disclosed in WO2012/027328 (PCT/US2011/048752), international filing date 23 Aug. 2011. In a further embodiment, the ABP of a combination of the invention, or a method or use thereof, is 119-222 or Hu119 or Hu119-222 antibody, e.g. as disclosed in WO2012/027328 (PCT/US2011/048752), international filing date 23 Aug. 2011. In a further embodiment, the ABP comprises CDRs or VH or VL or antibody sequences with at least 90% (e.g., 90%, 91%, 92%, 93%, 94%, 95%, 96%, 97%, 98%, or 99%, 100%) sequence identity to the sequences in this paragraph.

In another embodiment, the anti-OX40 ABP of a combination of the invention, or a method or use thereof, comprises the CDRs of the 119-43-1 antibody as disclosed in WO2013/028231 (PCT/US2012/024570), international filing date 9 Feb. 2012. In another embodiment, the anti-OX40 ABP of a combination of the invention, or a method or use thereof, comprises the CDRs of the 119-43-1 antibody as disclosed in WO2013/028231 (PCT/US2012/024570), international filing date 9 Feb. 2012. In a further embodiment, the anti-OX40 ABP of a combination of the invention, or a method or use thereof, comprises one of the VH and one of the VL regions of the 119-43-1 antibody. In a further embodiment, the anti-OX40 ABP of a combination of the invention, or a method or use thereof, comprises the VH and VL regions of the 119-43-1 antibody as disclosed in WO2013/028231 (PCT/US2012/024570), international filing date 9 Feb. 2012. In a further embodiment, the anti-OX40 ABP of a combination of the invention, or a method or use thereof, is 119-43-1 or 119-43-1 chimeric. In a further embodiment, the anti-OX40 ABP of a combination of the invention, or a method or use thereof, as disclosed in WO2013/028231 (PCT/US2012/024570), international filing date 9 Feb. 2012. In further embodiments, any one of the anti-OX40 ABPs described in this paragraph are humanized. In further embodiments, any one of the any one of the ABPs described in this paragraph are engineered to make a humanized antibody. In a further embodiment, the anti-OX40 ABP of a combination of the invention, or a method or use thereof, comprises CDRs or VH or VL or antibody sequences with at least 90% (e.g., 90%, 91%, 92%, 93%, 94%, 95%, 96%, 97%, 98%, or 99%, 100%) sequence identity to the sequences in this paragraph.

In another embodiment, further embodiment, any mouse or chimeric sequences of any anti-OX40 ABP of a combination of the invention, or a method or use thereof, are engineered to make a humanized antibody.

In one embodiment, the anti-OX40 ABP of a combination of the invention, or a method or use thereof, comprises: (a) a heavy chain variable region CDR1 comprising the amino acid sequence of SEQ ID NO: 1; (b) a heavy chain variable region CDR2 comprising the amino acid sequence of SEQ ID NO: 2; (c) a heavy chain variable region CDR3 comprising the amino acid sequence of SEQ ID NO. 3; (d) a light chain variable region CDR1 comprising the amino acid sequence of SEQ ID NO. 7; (e) a light chain variable region CDR2 comprising the amino acid sequence of SEQ ID NO. 8; and (f) a light chain variable region CDR3 comprising the amino acid sequence of SEQ ID NO. 9.

In another embodiment, the anti-OX40 ABP of a combination of the invention, or a method or use thereof, comprises: (a) a heavy chain variable region CDR1 comprising the amino acid sequence of SEQ ID NO: 13; (b) a heavy chain variable region CDR2 comprising the amino acid sequence of SEQ ID NO: 14; (c) a heavy chain variable region CDR3 comprising the amino acid sequence of SEQ ID NO. 15; (d) a light chain variable region CDR1 comprising the amino acid sequence of SEQ ID NO. 19; (e) a light chain variable region CDR2 comprising the amino acid sequence of SEQ ID NO. 20; and (f) a light chain variable region CDR3 comprising the amino acid sequence of SEQ ID NO. 21.

In another embodiment, the anti-OX40 ABP of a combination of the invention, or a method or use thereof, comprises: a heavy chain variable region CDR1 comprising the amino acid sequence of SEQ ID NO: 1 or 13; a heavy chain variable region CDR2 comprising the amino acid sequence of SEQ ID NO: 2 or 14; and/or a heavy chain variable region CDR3 comprising the amino acid sequence of SEQ ID NO: 3 or 15, or a heavy chain variable region CDR having 90 percent identity thereto.

In another embodiment, the anti-OX40 ABP of a combination of the invention, or a method or use thereof, comprises: a light chain variable region CDR1 comprising the amino acid sequence of SEQ ID NO: 7 or 19; a light chain variable region CDR2 comprising the amino acid sequence of SEQ ID NO: 8 or 20 and/or a light chain variable region CDR3 comprising the amino acid sequence of SEQ ID NO: 9 or 21, or a heavy chain variable region having 90 percent identity thereto.

In another embodiment, the anti-OX40 ABP of a combination of the invention, or a method or use thereof, comprises: a light chain variable region (“VL”) comprising the amino acid sequence of SEQ ID NO: 10, 11, 22 or 23, or an amino acid sequence with at least 90% (e.g., 90%, 91%, 92%, 93%, 94%, 95%, 96%, 97%, 98%, or 99%, 100%) sequence identity to the amino acid sequences of SEQ ID NO: 10, 11, 22 or 23. In another embodiment, the anti-OX40 ABP of a combination of the invention, or a method or use thereof, comprises a heavy chain variable region (“VH”) comprising the amino acid sequence of SEQ ID NO: 4, 5, 16 and 17, or an amino acid sequence with at least 90% (e.g., 90%, 91%, 92%, 93%, 94%, 95%, 96%, 97%, 98%, or 99%, 100%) sequence identity to the amino acid sequences of SEQ ID NO: 4, 5, 16 and 17. In another embodiment, the anti-OX40 ABP of a combination of the invention, or a method or use thereof, comprises a variable heavy sequence of SEQ ID NO:5 and a variable light sequence of SEQ ID NO: 11, or a sequence having 90 percent identity thereto. In another embodiment, the anti-OX40 ABP of a combination of the invention, or a method or use thereof, comprises a variable heavy sequence of SEQ ID NO:17 and a variable light sequence of SEQ ID NO: 23 or a sequence having 90 percent identity thereto.

In another embodiment, the anti-OX40 ABP of a combination of the invention, or a method or use thereof, comprises a variable light chain encoded by the nucleic acid sequence of SEQ ID NO: 12, or 24, or a nucleic acid sequence with at least 90% (e.g., 90%, 91%, 92%, 93%, 94%, 95%, 96%, 97%, 98%, or 99%, 100%) sequence identity to the nucleotide sequences of SEQ ID NO: 12 or 24. In another embodiment, the anti-OX40 ABP of a combination of the invention, or a method or use thereof, comprises a variable heavy chain encoded by a nucleic acid sequence of SEQ ID NO: 6 or 18, or a nucleic acid sequence with at least 90% (e.g., 90%, 91%, 92%, 93%, 94%, 95%, 96%, 97%, 98%, or 99%, 100%) sequence identity to nucleotide sequences of SEQ ID NO: 6 or 18.

Also provided herein are monoclonal antibodies. In one embodiment, the monoclonal antibodies comprise a variable light chain comprising the amino acid sequence of SEQ ID NO: 10 or 22, or an amino acid sequence with at least 90% (e.g., 90%, 91%, 92%, 93%, 94%, 95%, 96%, 97%, 98%, or 99%, 100%) sequence identity to the amino acid sequences of SEQ ID NO: 10 or 22. Further provided are monoclonal antibodies comprising a variable heavy chain comprising the amino acid sequence of SEQ ID NO: 4 or 16, or an amino acid sequence with at least 90% (e.g., 90%, 91%, 92%, 93%, 94%, 95%, 96%, 97%, 98%, or 99%, 100%) sequence identity to the amino acid sequences of SEQ ID NO: 4 or 16.

Also provided herein are monoclonal antibodies. In one embodiment, the monoclonal antibodies comprise a variable light chain comprising the amino acid sequence of SEQ ID NO: 11 or 23, or an amino acid sequence with at least 90% (e.g., 90%, 91%, 92%, 93%, 94%, 95%, 96%, 97%, 98%, or 99%, 100%) sequence identity to the amino acid sequences of SEQ ID NO: 11 or 23. Further provided are monoclonal antibodies comprising a variable heavy chain comprising the amino acid sequence of SEQ ID NO: 5 or 17, or an amino acid sequence with at least 90% (e.g., 90%, 91%, 92%, 93%, 94%, 95%, 96%, 97%, 98%, or 99%, 100%) sequence identity to the amino acid sequences of SEQ ID NO: 5 or 17.

Another embodiment of a combination of the invention, or a method or use thereof, includes CDRs, VH regions, and VL regions, and antibodies and nucleic acids encoding the same as disclosed in the below Sequence Listing.

| Heavy Chain of ANTIBODY 106-222: | |
|---|---|
| (SEQ ID NO: 48) | |
| QVQLVQSGSELKKPGASVKVSCKASGYTFTDYSMHWVRQAPGQGLKWMGWINTETGEP | |
| TYADDFKGRFVFSLDTSVSTAYLQISSLKAEDTAVYYCANPYYDYVSYYAMDYWGQGT | |
| TVTVSSASTKGPSVFPLAPSSKSTSGGTAALGCLVKDYFPEPVTVSWNSGALTSGVHT | |
| FPAVLQSSGLYSLSSVVTVPSSSLGTQTYICNVNHKPSNTKVDKKVEPKSCDKTHTCP | |
| PCPAPELLGGPSVFLFPPKPKDTLMISRTPEVTCVVVDVSHEDPEVKFNWYVDGVEVH | |
| NAKTKPREEQYNSTYRVVSVLTVLHQDWLNGKEYKCKVSNKALPAPIEKTISKAKGQP | |
| REPQVYTLPPSRDELTKNQVSLTCLVKGFYPSDIAVEWESNGQPENNYKTTPPVLDSD | |
| GSFFLYSKLTVDKSRWQQGNVFSCSVMHEALHNHYTQKSLSLSPGK | |
| Light Chain of ANTIBODY 106-222: | |
| (SEQ ID NO: 49) | |
| DIQMTQSPSSLSASVGDRVTITCKASQDVSTAVAWYQQKPGKAPKLLIYSASYLYTGV | |
| PSRFSGSGSGTDFTFTISSLQPEDIATYYCQQHYSTPRTFGQGTKLEIKRTVAAPSVF | |
| IFPPSDEQLKSGTASVVCLLNNFYPREAKVQWKVDNALQSGNSQESVTEQDSKDSTYS | |
| LSSTLTLSKADYEKHKVYACEVTHQGLSSPVTKSFNRGEC | |
| Heavy Chain Variable Region of ANTIBODY 106-222: | |
| (SEQ ID NO: 5) | |
| QVQLVQSGSELKKPGASVKVSCKASGYTFTDYSMHWVRQAPGQGLKWMGWINTETGEP | |
| TYADDFKGRFVFSLDTSVSTAYLQISSLKAEDTAVYYCANPYYDYVSYYAMDYWGQGT | |
| TVTVSS | |
| Light Chain Variable Region of ANTIBODY 106-222: | |
| (SEQ ID NO: 11) | |
| DIQMTQSPSSLSASVGDRVTITCKASQDVSTAVAWYQQKPGKAPKLLIYSASYLYTGV | |
| PSRFSGSGSGTDFTFTISSLQPEDIATYYCQQHYSTPRTFGQGTKLEIK | |
| CDR sequences of ANTIBODY 106-222: | |
| HC CDR1: | |
| (SEQ ID NO: 1) | |
| Asp Tyr Ser Met His | |
| HC CDR2: | |
| (SEQ ID NO: 2) | |
| Trp Ile Asn Thr Glu Thr Gly Glu Pro Thr Tyr Ala Asp Asp Phe Lys Gly | |
| HC CDR3: | |
| (SEQ ID NO: 3) | |
| Pro Tyr Tyr Asp Tyr Val Ser Tyr Tyr Ala Met Asp Tyr | |
| LC CDR1: | |
| (SEQ ID NO: 7) | |
| Lys Ala Ser Gln Asp Val Ser Thr Ala Val Ala | |
| LC CDR2: | |
| (SEQ ID NO: 8) | |
| Ser Ala Ser Tyr Leu Tyr Thr | |
| LC CDR3: | |
| (SEQ ID NO: 9) | |
| Gln Gln His Tyr Ser Thr Pro Arg Thr | |
| OX40 Antibody Sequence Listing | |
| <140> | UNKNOWN |
| <141> | 2014-02-24 |
| <150> | PCT/US2012/024570 |
| <151> | 2012-02-09 |
| <150> | PCT/US2011/048752 |
| <151> | 2011-08-23 |
| <160> | 47 |
| <170> | PatentIn version 3.5 |
| <210> | 1 |
| <211> | 5 |
| <212> | PRT |
| <213> | Mus sp. |
| <400> | 1 |
| Asp Tyr Ser Met His | |
| 1 5 | |
| <210> | 2 |
| <211> | 17 |
| <212> | PRT |
| <213> | Mus sp. |
| <400> | 2 |
| Trp Ile Asn Thr Glu Thr Gly Glu Pro Thr Tyr Ala Asp Asp Phe Lys | |
| 1 5 10 15 | |
| Gly | |
| <210> | 3 |
| <211> | 13 |
| <212> | PRT |
| <213> | Mus sp. |
| <400> | 3 |
| Pro Tyr Tyr Asp Tyr Val Ser Tyr Tyr Ala Met Asp Tyr | |
| 1 5 10 | |
| <210> | 4 |
| <211> | 122 |
| <212> | PRT |
| <213> | Mus sp. |
| <400> | 4 |
| Gln Ile Gln Leu Val Gln Ser Gly Pro Glu Leu Lys Lys Pro Gly Glu | |
| 1 5 10 15 | |
| Thr Val Lys Ile Ser Cys Lys Ala Ser Gly Tyr Thr Phe Thr Asp Tyr | |
| 20 25 30 | |
| Ser Met His Trp Val Lys Gln Ala Pro Gly Lys Gly Leu Lys Trp Met | |
| 35 40 45 | |
| Gly Trp Ile Asn Thr Glu Thr Gly Glu Pro Thr Tyr Ala Asp Asp Phe | |
| 50 55 60 | |
| Lys Gly Arg Phe Ala Phe Ser Leu Glu Thr Ser Ala Ser Thr Ala Tyr | |
| 65 70 75 80 | |
| Leu Gln Ile Asn Asn Leu Lys Asn Glu Asp Thr Ala Thr Tyr Phe Cys | |
| 85 90 95 | |
| Ala Asn Pro Tyr Tyr Asp Tyr Val Ser Tyr Tyr Ala Met Asp Tyr Trp | |
| 100 105 110 | |
| Gly His Gly Thr Ser Val Thr Val Ser Ser | |
| 115 120 | |
| <210> | 5 |
| <211> | 122 |
| <212> | PRT |
| <213> | Artificial Sequence |
| <220> | |
| <223> | Description of Artificial Sequence: Synthetic polypeptide |
| <400> | 5 |
| Gln Val Gln Leu Val Gln Ser Gly Ser Glu Leu Lys Lys Pro Gly Ala | |
| 1 5 10 15 | |
| Ser Val Lys Val Ser Cys Lys Ala Ser Gly Tyr Thr Phe Thr Asp Tyr | |
| 20 25 30 | |
| Ser Met His Trp Val Arg Gln Ala Pro Gly Gln Gly Leu Lys Trp Met | |
| 35 40 45 | |
| Gly Trp Ile Asn Thr Glu Thr Gly Glu Pro Thr Tyr Ala Asp Asp Phe | |
| 50 55 60 | |
| Lys Gly Arg Phe Val Phe Ser Leu Asp Thr Ser Val Ser Thr Ala Tyr | |
| 65 70 75 80 | |
| Leu Gln Ile Ser Ser Leu Lys Ala Glu Asp Thr Ala Val Tyr Tyr Cys | |
| 85 90 95 | |
| Ala Asn Pro Tyr Tyr Asp Tyr Val Ser Tyr Tyr Ala Met Asp Tyr Trp | |
| 100 105 110 | |
| Gly Gln Gly Thr Thr Val Thr Val Ser Ser | |
| 115 120 | |
| <210> | 6 |
| <211> | 458 |
| <212> | DNA |
| <213> | Artificial Sequence |
| <220> | |
| <223> | Description of Artificial Sequence: Synthetic polynucleotide |
| <400> | 6 |
| actagtacca ccatggcttg ggtgtggacc ttgctattcc tgatggcagc tgcccaaagt | |
| 60 | |
| atccaagcac aggttcagtt ggtgcagtct ggatctgagc tgaagaagcc tggagcctca | |
| 120 | |
| gtcaaggttt cctgcaaggc ttctggttat accttcacag actattcaat gcactgggtg | |
| 180 | |
| cgacaggctc caggacaagg tttaaagtgg atgggctgga taaacactga gactggtgag | |
| 240 | |
| ccaacatatg cagatgactt caagggacgg tttgtcttct ctttggacac ctctgtcagc | |
| 300 | |
| actgcctatt tgcagatcag cagcctcaaa gctgaggaca cggctgtgta ttactgtgct | |
| 360 | |
| aatccctact atgattacgt ctcttactat gctatggact actggggtca gggaaccacg | |
| 420 | |
| gtcaccgtct cctcaggtaa gaatggcctc tcaagctt | |
| 458 | |
| <210> | 7 |
| <211> | 11 |
| <212> | PRT |
| <213> | Mus sp. |
| <400> | 7 |
| Lys Ala Ser Gln Asp Val Ser Thr Ala Val Ala | |
| 1 5 10 | |
| <210> | 8 |
| <211> | 7 |
| <212> | PRT |
| <213> | Mus sp. |
| <400> | 8 |
| Ser Ala Ser Tyr Leu Tyr Thr | |
| 1 5 | |
| <210> | 9 |
| <211> | 9 |
| <212> | PRT |
| <213> | Mus sp. |
| <400> | 9 |
| Gln Gln His Tyr Ser Thr Pro Arg Thr | |
| 1 5 | |
| <210> | 10 |
| <211> | 107 |
| <212> | PRT |
| <213> | Mus sp. |
| <400> | 10 |
| Asp Ile Val Met Thr Gln Ser His Lys Phe Met Ser Thr Ser Val Arg | |
| 1 5 10 15 | |
| Asp Arg Val Ser Ile Thr Cys Lys Ala Ser Gln Asp Val Ser Thr Ala | |
| 20 25 30 | |
| Val Ala Trp Tyr Gln Gln Lys Pro Gly Gln Ser Pro Lys Leu Leu Ile | |
| 35 40 45 | |
| Tyr Ser Ala Ser Tyr Leu Tyr Thr Gly Val Pro Asp Arg Phe Thr Gly | |
| 50 55 60 | |
| Ser Gly Ser Gly Thr Asp Phe Thr Phe Thr Ile Ser Ser Val Gln Ala | |
| 65 70 75 80 | |
| Glu Asp Leu Ala Val Tyr Tyr Cys Gln Gln His Tyr Ser Thr Pro Arg | |
| 85 90 95 | |
| Thr Phe Gly Gly Gly Thr Lys Leu Glu Ile Lys | |
| 100 105 | |
| <210> | 11 |
| <211> | 107 |
| <212> | PRT |
| <213> | Artificial Sequence |
| <220> | |
| <223> | Description of Artificial Sequence: Synthetic polypeptide |
| <400> | 11 |
| Asp Ile Gln Met Thr Gln Ser Pro Ser Ser Leu Ser Ala Ser Val Gly | |
| 1 5 10 15 | |
| Asp Arg Val Thr Ile Thr Cys Lys Ala Ser Gln Asp Val Ser Thr Ala | |
| 20 25 30 | |
| Val Ala Trp Tyr Gln Gln Lys Pro Gly Lys Ala Pro Lys Leu Leu Ile | |
| 35 40 45 | |
| Tyr Ser Ala Ser Tyr Leu Tyr Thr Gly Val Pro Ser Arg Phe Ser Gly | |
| 50 55 60 | |
| Ser Gly Ser Gly Thr Asp Phe Thr Phe Thr Ile Ser Ser Leu Gln Pro | |
| 65 70 75 80 | |
| Glu Asp Ile Ala Thr Tyr Tyr Cys Gln Gln His Tyr Ser Thr Pro Arg | |
| 85 90 95 | |
| Thr Phe Gly Gln Gly Thr Lys Leu Glu Ile Lys | |
| 100 105 | |
| <210> | 12 |
| <211> | 416 |
| <212> | DNA |
| <213> | Artificial Sequence |
| <220> | |
| <223> | Description of Artificial Sequence: Synthetic polynucleotide |
| <400> | 12 |
| gctagcacca ccatggagtc acagattcag gtctttgtat tcgtgtttct ctggttgtct | |
| 60 | |
| ggtgttgacg gagacattca gatgacccag tctccatcct ccctgtccgc atcagtggga | |
| 120 | |
| gacagggtca ccatcacctg caaggccagt caggatgtga gtactgctgt agcctggtat | |
| 180 | |
| caacagaaac caggaaaagc ccctaaacta ctgatttact cggcatccta cctctacact | |
| 240 | |
| ggagtccctt cacgcttcag tggcagtgga tctgggacgg atttcacttt caccatcagc | |
| 300 | |
| agtctgcagc ctgaagacat tgcaacatat tactgtcagc aacattatag tactcctcgg | |
| 360 | |
| acgttcggtc agggcaccaa gctggaaatc aaacgtaagt agaatccaaa gaattc | |
| 416 | |
| <210> | 13 |
| <211> | 5 |
| <212> | PRT |
| <213> | Mus sp. |
| <400> | 13 |
| Ser His Asp Met Ser | |
| 1 5 | |
| <210> | 14 |
| <211> | 17 |
| <212> | PRT |
| <213> | Mus sp. |
| <400> | 14 |
| Ala Ile Asn Ser Asp Gly Gly Ser Thr Tyr Tyr Pro Asp Thr Met Glu | |
| 1 5 10 15 | |
| Arg | |
| <210> | 15 |
| <211> | 11 |
| <212> | PRT |
| <213> | Mus sp. |
| <400> | 15 |
| His Tyr Asp Asp Tyr Tyr Ala Trp Phe Ala Tyr | |
| 1 5 10 | |
| <210> | 16 |
| <211> | 120 |
| <212> | PRT |
| <213> | Mus sp. |
| <400> | 16 |
| Glu Val Gln Leu Val Glu Ser Gly Gly Gly Leu Val Gln Pro Gly Glu | |
| 1 5 10 15 | |
| Ser Leu Lys Leu Ser Cys Glu Ser Asn Glu Tyr Glu Phe Pro Ser His | |
| 20 25 30 | |
| Asp Met Ser Trp Val Arg Lys Thr Pro Glu Lys Arg Leu Glu Leu Val | |
| 35 40 45 | |
| Ala Ala Ile Asn Ser Asp Gly Gly Ser Thr Tyr Tyr Pro Asp Thr Met | |
| 50 55 60 | |
| Glu Arg Arg Phe Ile Ile Ser Arg Asp Asn Thr Lys Lys Thr Leu Tyr | |
| 65 70 75 80 | |
| Leu Gln Met Ser Ser Leu Arg Ser Glu Asp Thr Ala Leu Tyr Tyr Cys | |
| 85 90 95 | |
| Ala Arg His Tyr Asp Asp Tyr Tyr Ala Trp Phe Ala Tyr Trp Gly Gln | |
| 100 105 110 | |
| Gly Thr Leu Val Thr Val Ser Ala | |
| 115 120 | |
| <210> | 17 |
| <211> | 120 |
| <212> | PRT |
| <213> | Artificial Sequence |
| <220> | |
| <223> | Description of Artificial Sequence: Synthetic polypeptide |
| <400> | 17 |
| Glu Val Gln Leu Val Glu Ser Gly Gly Gly Leu Val Gln Pro Gly Gly | |
| 1 5 10 15 | |
| Ser Leu Arg Leu Ser Cys Ala Ala Ser Glu Tyr Glu Phe Pro Ser His | |
| 20 25 30 | |
| Asp Met Ser Trp Val Arg Gln Ala Pro Gly Lys Gly Leu Glu Leu Val | |
| 35 40 45 | |
| Ala Ala Ile Asn Ser Asp Gly Gly Ser Thr Tyr Tyr Pro Asp Thr Met | |
| 50 55 60 | |
| Glu Arg Arg Phe Thr Ile Ser Arg Asp Asn Ala Lys Asn Ser Leu Tyr | |
| 65 70 75 80 | |
| Leu Gln Met Asn Ser Leu Arg Ala Glu Asp Thr Ala Val Tyr Tyr Cys | |
| 85 90 95 | |
| Ala Arg His Tyr Asp Asp Tyr Tyr Ala Trp Phe Ala Tyr Trp Gly Gln | |
| 100 105 110 | |
| Gly Thr Met Val Thr Val Ser Ser | |
| 115 120 | |
| <210> | 18 |
| <211> | 451 |
| <212> | DNA |
| <213> | Artificial Sequence |
| <220> | |
| <223> | Description of Artificial Sequence: Synthetic polynucleotide |
| <400> | 18 |
| actagtacca ccatggactt cgggctcagc ttggttttcc ttgtccttat tttaaaaagt | |
| 60 | |
| gtacagtgtg aggtgcagct ggtggagtct gggggaggct tagtgcagcc tggagggtcc | |
| 120 | |
| ctgagactct cctgtgcagc ctctgaatac gagttccctt cccatgacat gtcttgggtc | |
| 180 | |
| cgccaggctc cggggaaggg gctggagttg gtcgcagcca ttaatagtga tggtggtagc | |
| 240 | |
| acctactatc cagacaccat ggagagacga ttcaccatct ccagagacaa tgccaagaac | |
| 300 | |
| tcactgtacc tgcaaatgaa cagtctgagg gccgaggaca cagccgtgta ttactgtgca | |
| 360 | |
| agacactatg atgattacta cgcctggttt gcttactggg gccaagggac tatggtcact | |
| 420 | |
| gtctcttcag gtgagtccta acttcaagct t | |
| 451 | |
| <210> | 19 |
| <211> | 15 |
| <212> | PRT |
| <213> | Mus sp. |
| <400> | 19 |
| Arg Ala Ser Lys Ser Val Ser Thr Ser Gly Tyr Ser Tyr Met His | |
| 1 5 10 15 | |
| <210> | 20 |
| <211> | 7 |
| <212> | PRT |
| <213> | Mus sp. |
| <400> | 20 |
| Leu Ala Ser Asn Leu Glu Ser | |
| 1 5 | |
| <210> | 21 |
| <211> | 9 |
| <212> | PRT |
| <213> | Mus sp. |
| <400> | 21 |
| Gln His Ser Arg Glu Leu Pro Leu Thr | |
| 1 5 | |
| <210> | 22 |
| <211> | 111 |
| <212> | PRT |
| <213> | Mus sp. |
| <400> | 22 |
| Asp Ile Val Leu Thr Gln Ser Pro Ala Ser Leu Ala Val Ser Leu Gly | |
| 1 5 10 15 | |
| Gln Arg Ala Thr Ile Ser Cys Arg Ala Ser Lys Ser Val Ser Thr Ser | |
| 20 25 30 | |
| Gly Tyr Ser Tyr Met His Trp Tyr Gln Gln Lys Pro Gly Gln Pro Pro | |
| 35 40 45 | |
| Lys Leu Leu Ile Tyr Leu Ala Ser Asn Leu Glu Ser Gly Val Pro Ala | |
| 50 55 60 | |
| Arg Phe Ser Gly Ser Gly Ser Gly Thr Asp Phe Thr Leu Asn Ile His | |
| 65 70 75 80 | |
| Pro Val Glu Glu Glu Asp Ala Ala Thr Tyr Tyr Cys Gln His Ser Arg | |
| 85 90 95 | |
| Glu Leu Pro Leu Thr Phe Gly Ala Gly Thr Lys Leu Glu Leu Lys | |
| 100 105 110 | |
| <210> | 23 |
| <211> | 111 |
| <212> | PRT |
| <213> | Artificial Sequence |
| <220> | |
| <223> | Description of Artificial Sequence: Synthetic polypeptide |
| <400> | 23 |
| Glu Ile Val Leu Thr Gln Ser Pro Ala Thr Leu Ser Leu Ser Pro Gly | |
| 1 5 10 15 | |
| Glu Arg Ala Thr Leu Ser Cys Arg Ala Ser Lys Ser Val Ser Thr Ser | |
| 20 25 30 | |
| Gly Tyr Ser Tyr Met His Trp Tyr Gln Gln Lys Pro Gly Gln Ala Pro | |
| 35 40 45 | |
| Arg Leu Leu Ile Tyr Leu Ala Ser Asn Leu Glu Ser Gly Val Pro Ala | |
| 50 55 60 | |
| Arg Phe Ser Gly Ser Gly Ser Gly Thr Asp Phe Thr Leu Thr Ile Ser | |
| 65 70 75 80 | |
| Ser Leu Glu Pro Glu Asp Phe Ala Val Tyr Tyr Cys Gln His Ser Arg | |
| 85 90 95 | |
| Glu Leu Pro Leu Thr Phe Gly Gly Gly Thr Lys Val Glu Ile Lys | |
| 100 105 110 | |
| <210> | 24 |
| <211> | 428 |
| <212> | DNA |
| <213> | Artificial Sequence |
| <220> | |
| <223> | Description of Artificial Sequence: Synthetic polynucleotide |
| <400> | 24 |
| gctagcacca ccatggagac agacacactc ctgttatggg tactgctgct ctgggttcca | |
| 60 | |
| ggttccactg gtgaaattgt gctgacacag tctcctgcta ccttatcttt gtctccaggg | |
| 120 | |
| gaaagggcca ccctctcatg cagggccagc aaaagtgtca gtacatctgg ctatagttat | |
| 180 | |
| atgcactggt accaacagaa accaggacag gctcccagac tcctcatcta tcttgcatcc | |
| 240 | |
| aacctagaat ctggggtccc tgccaggttc agtggcagtg ggtctgggac agacttcacc | |
| 300 | |
| ctcaccatca gcagcctaga gcctgaggat tttgcagttt attactgtca gcacagtagg | |
| 360 | |
| gagcttccgc tcacgttcgg cggagggacc aaggtcgaga tcaaacgtaa gtacactttt | |
| 420 | |
| ctgaattc | |
| 428 | |
| <210> | 25 |
| <211> | 5 |
| <212> | PRT |
| <213> | Mus sp. |
| <400> | 25 |
| Asp Ala Trp Met Asp | |
| 1 5 | |
| <210> | 26 |
| <211> | 19 |
| <212> | PRT |
| <213> | Mus sp. |
| <400> | 26 |
| Glu Ile Arg Ser Lys Ala Asn Asn His Ala Thr Tyr Tyr Ala Glu Ser | |
| 1 5 10 15 | |
| Val Asn Gly | |
| <210> | 27 |
| <211> | 8 |
| <212> | PRT |
| <213> | Mus sp. |
| <400> | 27 |
| Gly Glu Val Phe Tyr Phe Asp Tyr | |
| 1 5 | |
| <210> | 28 |
| <211> | 414 |
| <212> | DNA |
| <213> | Mus sp. |
| <400> | 28 |
| atgtacttgg gactgaacta tgtattcata gtttttctct taaatggtgt ccagagtgaa | |
| 60 | |
| gtgaagcttg aggagtctgg aggaggcttg gtgcaacctg gaggatccat gaaactctct | |
| 120 | |
| tgtgctgcct ctggattcac ttttagtgac gcctggatgg actgggtccg ccagtctcca | |
| 180 | |
| gagaaggggc ttgagtgggt tgctgaaatt agaagcaaag ctaataatca tgcaacatac | |
| 240 | |
| tatgctgagt ctgtgaatgg gaggttcacc atctcaagag atgattccaa aagtagtgtc | |
| 300 | |
| tacctgcaaa tgaacagctt aagagctgaa gacactggca tttattactg tacgtggggg | |
| 360 | |
| gaagtgttct actttgacta ctggggccaa ggcaccactc tcacagtctc ctca | |
| 414 | |
| <210> | 29 |
| <211> | 138 |
| <212> | PRT |
| <213> | Mus sp. |
| <400> | 29 |
| Met Tyr Leu Gly Leu Asn Tyr Val Phe Ile Val Phe Leu Leu Asn Gly | |
| 1 5 10 15 | |
| Val Gln Ser Glu Val Lys Leu Glu Glu Ser Gly Gly Gly Leu Val Gln | |
| 20 25 30 | |
| Pro Gly Gly Ser Met Lys Leu Ser Cys Ala Ala Ser Gly Phe Thr Phe | |
| 35 40 45 | |
| Ser Asp Ala Trp Met Asp Trp Val Arg Gln Ser Pro Glu Lys Gly Leu | |
| 50 55 60 | |
| Glu Trp Val Ala Glu Ile Arg Ser Lys Ala Asn Asn His Ala Thr Tyr | |
| 65 70 75 80 | |
| Tyr Ala Glu Ser Val Asn Gly Arg Phe Thr Ile Ser Arg Asp Asp Ser | |
| 85 90 95 | |
| Lys Ser Ser Val Tyr Leu Gln Met Asn Ser Leu Arg Ala Glu Asp Thr | |
| 100 105 110 | |
| Gly Ile Tyr Tyr Cys Thr Trp Gly Glu Val Phe Tyr Phe Asp Tyr Trp | |
| 115 120 125 | |
| Gly Gln Gly Thr Thr Leu Thr Val Ser Ser | |
| 130 135 | |
| <210> | 30 |
| <211> | 448 |
| <212> | DNA |
| <213> | Artificial Sequence |
| <220> | |
| <223> | Description of Artificial Sequence: Synthetic polynucleotide |
| <400> | 30 |
| actagtacca ccatgtactt gggactgaac tatgtattca tagtttttct cttaaatggt | |
| 60 | |
| gtccagagtg aagtgaagct ggaggagtct ggaggaggct tggtgcaacc tggaggatcc | |
| 120 | |
| atgaaactct cttgtgctgc ctctggattc acttttagtg acgcctggat ggactgggtc | |
| 180 | |
| cgccagtctc cagagaaggg gcttgagtgg gttgctgaaa ttagaagcaa agctaataat | |
| 240 | |
| catgcaacat actatgctga gtctgtgaat gggaggttca ccatctcaag agatgattcc | |
| 300 | |
| aaaagtagtg tctacctgca aatgaacagc ttaagagctg aagacactgg catttattac | |
| 360 | |
| tgtacgtggg gggaagtgtt ctactttgac tactggggcc aaggcaccac tctcacagtc | |
| 420 | |
| tcctcaggtg agtccttaaa acaagctt | |
| 448 | |
| <210> | 31 |
| <211> | 138 |
| <212> | PRT |
| <213> | Artificial Sequence |
| <220> | |
| <223> | Description of Artificial Sequence: Synthetic polypeptide |
| <400> | 31 |
| Met Tyr Leu Gly Leu Asn Tyr Val Phe Ile Val Phe Leu Leu Asn Gly | |
| 1 5 10 15 | |
| Val Gln Ser Glu Val Lys Leu Glu Glu Ser Gly Gly Gly Leu Val Gln | |
| 20 25 30 | |
| Pro Gly Gly Ser Met Lys Leu Ser Cys Ala Ala Ser Gly Phe Thr Phe | |
| 35 40 45 | |
| Ser Asp Ala Trp Met Asp Trp Val Arg Gln Ser Pro Glu Lys Gly Leu | |
| 50 55 60 | |
| Glu Trp Val Ala Glu Ile Arg Ser Lys Ala Asn Asn His Ala Thr Tyr | |
| 65 70 75 80 | |
| Tyr Ala Glu Ser Val Asn Gly Arg Phe Thr Ile Ser Arg Asp Asp Ser | |
| 85 90 95 | |
| Lys Ser Ser Val Tyr Leu Gln Met Asn Ser Leu Arg Ala Glu Asp Thr | |
| 100 105 110 | |
| Gly Ile Tyr Tyr Cys Thr Trp Gly Glu Val Phe Tyr Phe Asp Tyr Trp | |
| 115 120 125 | |
| Gly Gln Gly Thr Thr Leu Thr Val Ser Ser | |
| 130 135 | |
| <210> | 32 |
| <211> | 11 |
| <212> | PRT |
| <213> | Mus sp. |
| <400> | 32 |
| Lys Ser Ser Gln Asp Ile Asn Lys Tyr Ile Ala | |
| 1 5 10 | |
| <210> | 33 |
| <211> | 7 |
| <212> | PRT |
| <213> | Mus sp. |
| <400> | 33 |
| Tyr Thr Ser Thr Leu Gln Pro | |
| 1 5 | |
| <210> | 34 |
| <211> | 8 |
| <212> | PRT |
| <213> | Mus sp. |
| <400> | 34 |
| Leu Gln Tyr Asp Asn Leu Leu Thr | |
| 1 5 | |
| <210> | 35 |
| <211> | 378 |
| <212> | DNA |
| <213> | Mus sp. |
| <400> | 35 |
| atgagaccgt ctattcagtt cctggggctc ttgttgttct ggcttcatgg tgctcagtgt | |
| 60 | |
| gacatccaga tgacacagtc tccatcctca ctgtctgcat ctctgggagg caaagtcacc | |
| 120 | |
| atcacttgca agtcaagcca agacattaac aagtatatag cttggtacca acacaagcct | |
| 180 | |
| ggaaaaggtc ctaggctgct catacattac acatctacat tacagccagg catcccatca | |
| 240 | |
| aggttcagtg gaagtgggtc tgggagagat tattccttca gcatcagcaa cctggagcct | |
| 300 | |
| gaagatattg caacttatta ttgtctacag tatgataatc ttctcacgtt cggtgctggg | |
| 360 | |
| accaagctgg agctgaaa | |
| 378 | |
| <210> | 36 |
| <211> | 126 |
| <212> | PRT |
| <213> | Mus sp. |
| <400> | 36 |
| Met Arg Pro Ser Ile Gln Phe Leu Gly Leu Leu Leu Phe Trp Leu His | |
| 1 5 10 15 | |
| Gly Ala Gln Cys Asp Ile Gln Met Thr Gln Ser Pro Ser Ser Leu Ser | |
| 20 25 30 | |
| Ala Ser Leu Gly Gly Lys Val Thr Ile Thr Cys Lys Ser Ser Gln Asp | |
| 35 40 45 | |
| Ile Asn Lys Tyr Ile Ala Trp Tyr Gln His Lys Pro Gly Lys Gly Pro | |
| 50 55 60 | |
| Arg Leu Leu Ile His Tyr Thr Ser Thr Leu Gln Pro Gly Ile Pro Ser | |
| 65 70 75 80 | |
| Arg Phe Ser Gly Ser Gly Ser Gly Arg Asp Tyr Ser Phe Ser Ile Ser | |
| 85 90 95 | |
| Asn Leu Glu Pro Glu Asp Ile Ala Thr Tyr Tyr Cys Leu Gln Tyr Asp | |
| 100 105 110 | |
| Asn Leu Leu Thr Phe Gly Ala Gly Thr Lys Leu Glu Leu Lys | |
| 115 120 125 | |
| <210> | 37 |
| <211> | 413 |
| <212> | DNA |
| <213> | Artificial Sequence |
| <220> | |
| <223> | Description of Artificial Sequence: Synthetic polynucleotide |
| <400> | 37 |
| gctagcacca ccatgagacc gtctattcag ttcctggggc tcttgttgtt ctggcttcat | |
| 60 | |
| ggtgctcagt gtgacatcca gatgacacag tctccatcct cactgtctgc atctctggga | |
| 120 | |
| ggcaaagtca ccatcacttg caagtcaagc caagacatta acaagtatat agcttggtac | |
| 180 | |
| caacacaagc ctggaaaagg tcctaggctg ctcatacatt acacatctac attacagcca | |
| 240 | |
| ggcatcccat caaggttcag tggaagtggg tctgggagag attattcctt cagcatcagc | |
| 300 | |
| aacctggagc ctgaagatat tgcaacttat tattgtctac agtatgataa tcttctcacg | |
| 360 | |
| ttcggtgctg ggaccaagct ggagctgaaa cgtaagtaca cttttctgaa ttc | |
| 413 | |
| <210> | 38 |
| <211> | 126 |
| <212> | PRT |
| <213> | Artificial Sequence |
| <220> | |
| <223> | Description of Artificial Sequence: Synthetic polypeptide |
| <400> | 38 |
| Met Arg Pro Ser Ile Gln Phe Leu Gly Leu Leu Leu Phe Trp Leu His | |
| 1 5 10 15 | |
| Gly Ala Gln Cys Asp Ile Gln Met Thr Gln Ser Pro Ser Ser Leu Ser | |
| 20 25 30 | |
| Ala Ser Leu Gly Gly Lys Val Thr Ile Thr Cys Lys Ser Ser Gln Asp | |
| 35 40 45 | |
| Ile Asn Lys Tyr Ile Ala Trp Tyr Gln His Lys Pro Gly Lys Gly Pro | |
| 50 55 60 | |
| Arg Leu Leu Ile His Tyr Thr Ser Thr Leu Gln Pro Gly Ile Pro Ser | |
| 65 70 75 80 | |
| Arg Phe Ser Gly Ser Gly Ser Gly Arg Asp Tyr Ser Phe Ser Ile Ser | |
| 85 90 95 | |
| Asn Leu Glu Pro Glu Asp Ile Ala Thr Tyr Tyr Cys Leu Gln Tyr Asp | |
| 100 105 110 | |
| Asn Leu Leu Thr Phe Gly Ala Gly Thr Lys Leu Glu Leu Lys | |
| 115 120 125 | |
| <210> | 39 |
| <211> | 20 |
| <212> | DNA |
| <213> | Artificial Sequence |
| <220> | |
| <223> | Description of Artificial Sequence: Synthetic primer |
| <400> | 39 |
| cgctgttttg acctccatag | |
| 20 | |
| <210> | 40 |
| <211> | 20 |
| <212> | DNA |
| <213> | Artificial Sequence |
| <220> | |
| <223> | Description of Artificial Sequence: Synthetic primer |
| <400> | 40 |
| tgaaagatga gctggaggac | |
| 20 | |
| <210> | 41 |
| <211> | 20 |
| <212> | DNA |
| <213> | Artificial Sequence |
| <220> | |
| <223> | Description of Artificial Sequence: Synthetic primer |
| <400> | 41 |
| ctttcttgtc caccttggtg | |
| 20 | |
| <210> | 42 |
| <211> | 19 |
| <212> | DNA |
| <213> | Artificial Sequence |
| <220> | |
| <223> | Description of Artificial Sequence: Synthetic primer |
| <400> | 42 |
| gctgtcctac agtcctcag | |
| 19 | |
| <210> | 43 |
| <211> | 18 |
| <212> | DNA |
| <213> | Artificial Sequence |
| <220> | |
| <223> | Description of Artificial Sequence: Synthetic primer |
| <400> | 43 |
| acgtgccaag catcctcg | |
| 18 | |
| <210> | 44 |
| <211> | 1407 |
| <212> | DNA |
| <213> | Artificial Sequence |
| <220> | |
| <223> | Description of Artificial Sequence: Synthetic polynucleotide |
| <400> | 44 |
| atgtacttgg gactgaacta tgtattcata gtttttctct taaatggtgt ccagagtgaa | |
| 60 | |
| gtgaagctgg aggagtctgg aggaggcttg gtgcaacctg gaggatccat gaaactctct | |
| 120 | |
| tgtgctgcct ctggattcac ttttagtgac gcctggatgg actgggtccg ccagtctcca | |
| 180 | |
| gagaaggggc ttgagtgggt tgctgaaatt agaagcaaag ctaataatca tgcaacatac | |
| 240 | |
| tatgctgagt ctgtgaatgg gaggttcacc atctcaagag atgattccaa aagtagtgtc | |
| 300 | |
| tacctgcaaa tgaacagctt aagagctgaa gacactggca tttattactg tacgtggggg | |
| 360 | |
| gaagtgttct actttgacta ctggggccaa ggcaccactc tcacagtctc ctcagcctcc | |
| 420 | |
| accaagggcc catcggtctt ccccctggca ccctcctcca agagcacctc tgggggcaca | |
| 480 | |
| gcggccctgg gctgcctggt caaggactac ttccccgaac cggtgacggt gtcgtggaac | |
| 540 | |
| tcaggcgccc tgaccagcgg cgtgcacacc ttcccggctg tcctacagtc ctcaggactc | |
| 600 | |
| tactccctca gcagcgtggt gaccgtgccc tccagcagct tgggcaccca gacctacatc | |
| 660 | |
| tgcaacgtga atcacaagcc cagcaacacc aaggtggaca agaaagttga gcccaaatct | |
| 720 | |
| tgtgacaaaa ctcacacatg cccaccgtgc ccagcacctg aactcctggg gggaccgtca | |
| 780 | |
| gtcttcctct tccccccaaa acccaaggac accctcatga tctcccggac ccctgaggtc | |
| 840 | |
| acatgcgtgg tggtggacgt gagccacgaa gaccctgagg tcaagttcaa ctggtacgtg | |
| 900 | |
| gacggcgtgg aggtgcataa tgccaagaca aagccgcggg aggagcagta caacagcacg | |
| 960 | |
| taccgtgtgg tcagcgtcct caccgtcctg caccaggact ggctgaatgg caaggagtac | |
| 1020 | |
| aagtgcaagg tctccaacaa agccctccca gcccccatcg agaaaaccat ctccaaagcc | |
| 1080 | |
| aaagggcagc cccgagaacc acaggtgtac accctgcccc catcccggga tgagctgacc | |
| 1140 | |
| aagaaccagg tcagcctgac ctgcctggtc aaaggcttct atcccagcga catcgccgtg | |
| 1200 | |
| gagtgggaga gcaatgggca gccggagaac aactacaaga ccacgcctcc cgtgctggac | |
| 1260 | |
| tccgacggct ccttcttcct ctacagcaag ctcaccgtgg acaagagcag gtggcagcag | |
| 1320 | |
| gggaacgtct tctcatgctc cgtgatgcat gaggctctgc acaaccacta cacgcagaag | |
| 1380 | |
| agcctctccc tgtctccggg taaatga | |
| 1407 | |
| <210> | 45 |
| <211> | 469 |
| <212> | PRT |
| <213> | Artificial Sequence |
| <220> | |
| <223> | Description of Artificial Sequence: Synthetic polypeptide |
| <400> | 45 |
| Met Tyr Leu Gly Leu Asn Tyr Val Phe Ile Val Phe Leu Leu Asn Gly | |
| 1 5 10 15 | |
| Val Gln Ser Glu Val Lys Leu Glu Glu Ser Gly Gly Gly Leu Val Gln | |
| 20 25 30 | |
| Pro Gly Gly Ser Met Lys Leu Ser Cys Ala Ala Ser Gly Phe Thr Phe | |
| 35 40 45 | |
| Ser Asp Ala Trp Met Asp Trp Val Arg Gln Ser Pro Glu Lys Gly Leu | |
| 50 55 60 | |
| Glu Trp Val Ala Glu Ile Arg Ser Lys Ala Asn Asn His Ala Thr Tyr | |
| 65 70 75 80 | |
| Tyr Ala Glu Ser Val Asn Gly Arg Phe Thr Ile Ser Arg Asp Asp Ser | |
| 85 90 95 | |
| Lys Ser Ser Val Tyr Leu Gln Met Asn Ser Leu Arg Ala Glu Asp Thr | |
| 100 105 110 | |
| Gly Ile Tyr Tyr Cys Thr Trp Gly Glu Val Phe Tyr Phe Asp Tyr Trp | |
| 115 120 125 | |
| Gly Gln Gly Thr Thr Leu Thr Val Ser Ser Ala Ser Thr Lys Gly Pro | |
| 130 135 140 | |
| Ser Val Phe Pro Leu Ala Pro Ser Ser Lys Ser Thr Ser Gly Gly Thr | |
| 145 150 155 160 | |
| Ala Ala Leu Gly Cys Leu Val Lys Asp Tyr Phe Pro Glu Pro Val Thr | |
| 165 170 175 | |
| Val Ser Trp Asn Ser Gly Ala Leu Thr Ser Gly Val His Thr Phe Pro | |
| 180 185 190 | |
| Ala Val Leu Gln Ser Ser Gly Leu Tyr Ser Leu Ser Ser Val Val Thr | |
| 195 200 205 | |
| Val Pro Ser Ser Ser Leu Gly Thr Gln Thr Tyr Ile Thr Cys Asn Val | |
| 210 215 220 | |
| Asn His Lys Pro Ser Asn Thr Lys Val Asp Lys Lys Val Glu Pro Lys | |
| 225 230 235 240 | |
| Ser Cys Asp Lys Thr His Thr Cys Pro Pro Cys Pro Ala Pro Glu Leu | |
| 245 250 255 | |
| Leu Gly Gly Pro Ser Val Phe Leu Phe Pro Pro Lys Pro Lys Asp Thr | |
| 260 265 270 | |
| Leu Met Ile Ser Arg Thr Pro Glu Val Thr Cys Val Val Val Asp Val | |
| 275 280 285 | |
| Ser His Glu Asp Pro Glu Val Lys Phe Asn Trp Tyr Val Asp Gly Val | |
| 290 295 300 | |
| Glu Val His Asn Ala Lys Thr Lys Pro Arg Glu Glu Gln Tyr Asn Ser | |
| 305 310 315 320 | |
| Thr Tyr Arg Val Val Ser Val Leu Thr Val Leu His Gln Asp Trp Leu | |
| 325 330 335 | |
| Asn Gly Lys Glu Tyr Lys Cys Lys Val Ser Asn Lys Ala Leu Pro Ala | |
| 340 345 350 | |
| Pro Ile Glu Lys Thr Ile Ser Lys Ala Lys Gly Gln Pro Arg Glu Pro | |
| 355 360 365 | |
| Gln Val Tyr Thr Leu Pro Pro Ser Arg Asp Glu Leu Thr Lys Asn Gln | |
| 370 375 380 | |
| Val Ser Leu Thr Cys Leu Val Lys Gly Phe Tyr Pro Ser Asp Ile Ala | |
| 385 390 395 400 | |
| Val Glu Trp Glu Ser Asn Gly Gln Pro Glu Asn Asn Tyr Lys Thr Thr | |
| 405 410 415 | |
| Pro Pro Val Leu Asp Ser Asp Gly Ser Phe Phe Leu Tyr Ser Lys Leu | |
| 420 425 430 | |
| Thr Val Asp Lys Ser Arg Trp Gln Gln Gly Asn Val Phe Ser Cys Ser | |
| 435 440 445 | |
| Val Met His Glu Ala Leu His Asn His Tyr Thr Gln Lys Ser Leu Ser | |
| 450 455 460 | |
| Leu Ser Pro Gly Lys | |
| 465 | |
| <210> | 46 |
| <211> | 702 |
| <212> | DNA |
| <213> | Artificial Sequence |
| <220> | |
| <223> | Description of Artificial Sequence: Synthetic polynucleotide |
| <400> | 46 |
| atgagaccgt ctattcagtt cctggggctc ttgttgttct ggcttcatgg tgctcagtgt | |
| 60 | |
| gacatccaga tgacacagtc tccatcctca ctgtctgcat ctctgggagg caaagtcacc | |
| 120 | |
| atcacttgca agtcaagcca agacattaac aagtatatag cttggtacca acacaagcct | |
| 180 | |
| ggaaaaggtc ctaggctgct catacattac acatctacat tacagccagg catcccatca | |
| 240 | |
| aggttcagtg gaagtgggtc tgggagagat tattccttca gcatcagcaa cctggagcct | |
| 300 | |
| gaagatattg caacttatta ttgtctacag tatgataatc ttctcacgtt cggtgctggg | |
| 360 | |
| accaagctgg agctgaaacg aactgtggct gcaccatctg tcttcatctt cccgccatct | |
| 420 | |
| gatgagcagt tgaaatctgg aactgcctct gttgtgtgcc tgctgaataa cttctatccc | |
| 480 | |
| agagaggcca aagtacagtg gaaggtggat aacgccctcc aatcgggtaa ctcccaggag | |
| 540 | |
| agtgtcacag agcaggacag caaggacagc acctacagcc tcagcagcac cctgacgctg | |
| 600 | |
| agcaaagcag actacgagaa acacaaagtc tacgcctgcg aagtcaccca tcagggcctg | |
| 660 | |
| agctcgcccg tcacaaagag cttcaacagg ggagagtgtt ag | |
| 702 | |
| <210> | 47 |
| <211> | 233 |
| <212> | PRT |
| <213> | Artificial Sequence |
| <220> | |
| <223> | Description of Artificial Sequence: Synthetic polypeptide |
| <400> | 47 |

```
Met Arg Pro Ser Ile Gln Phe Leu Gly Leu Leu Leu Phe Trp Leu His
1 5 10 15
Gly Ala Gln Cys Asp Ile Gln Met Thr Gln Ser Pro Ser Ser Leu Ser
                        20 25 30
Ala Ser Leu Gly Gly Lys Val Thr Ile Thr Cys Lys Ser Ser Gln Asp
                35 40 45
Ile Asn Lys Tyr Ile Ala Trp Tyr Gln His Lys Pro Gly Lys Gly Pro
        50 55 60
Arg Leu Leu Ile His Tyr Thr Ser Thr Leu Gln Pro Gly Ile Pro Ser
65 70 75 80
Arg Phe Ser Gly Ser Gly Ser Gly Arg Asp Tyr Ser Phe Ser Ile Ser
                                85 90 95
Asn Leu Glu Pro Glu Asp Ile Ala Thr Tyr Tyr Cys Leu Gln Tyr Asp
                        100 105 110
Asn Leu Leu Thr Phe Gly Ala Gly Thr Lys Leu Glu Leu Lys Arg Thr
                115 120 125
Val Ala Ala Pro Ser Val Phe Ile Phe Pro Pro Ser Asp Glu Gln Leu
        130 135 140
Lys Ser Gly Thr Ala Ser Val Val Cys Leu Leu Asn Asn Phe Tyr Pro
145 150 155 160
Arg Glu Ala Lys Val Gln Trp Lys Val Asp Asn Ala Leu Gln Ser Gly
                                165 170 175
Asn Ser Gln Glu Ser Val Thr Glu Gln Asp Ser Lys Asp Ser Thr Tyr
                        180 185 190
Ser Leu Ser Ser Thr Leu Thr Leu Ser Lys Ala Asp Tyr Glu Lys His
                195 200 205
Lys Val Tyr Ala Cys Glu Val Thr His Gln Gly Leu Ser Ser Pro Val
        210 215 220
Thr Lys Ser Phe Asn Arg Gly Glu Cys
225 230
```

SEQ ID NOS:39-43 are the sequences of oligonucleotides used for PCR amplification and sequencing of Ch119-43-1 heavy and light chain cDNA.

SEQ ID NO:44 provides the nucleotide sequence of the coding region of gamma-1 heavy chain in pCh119-43-1 along with the deduced amino acid sequence (SEQ ID NO:45). Amino acid residues are shown in single letter code.

SEQ ID NO:46 provides the nucleotide sequence of the coding region of kappa light chain in pCh119-43-1 along with the deduced amino acid sequence (SEQ ID NO:47). Amino acid residues are shown in single letter code.

### CTLA-4 Antigen Binding Proteins

The combinations, and methods and uses thereof, of the invention comprise anti-CTLA-4 antigen binding proteins that bind CTLA-4, such as antagonists molecules (such as antibodies) that block binding with a CTLA-4 ligand such as CD80/CD86.

An OX40 antibody, e.g., an antibody described herein, can be used in combination with an antibody (e.g., antagonist antibody) against CTLA-4 (e.g., human CTLA-4). For example, an OX40 antibody can be used in combination with ipilimumab or tremelimumab.

In an aspect, the equilibrium dissociation constant (KD) of the anti-CTLA-4 ABP of a combination of the invention, or a method or use thereof, and CTLA-4, preferably human CTLA-4, interaction is 100 nM or less, 10 nM or less, 2 nM or less or 1 nM or less. Alternatively the KD may be between 5 and 10 nM; or between 1 and 2 nM. The KD may be between 1 pM and 500 pM; or between 500 pM and 1 nM. A skilled person will appreciate that the smaller the KD numerical value, the stronger the binding. The reciprocal of KD (i.e. 1/KD) is the equilibrium association constant (KA) having units M-1. A skilled person will appreciate that the larger the KA numerical value, the stronger the binding.

The dissociation rate constant (kd) or “off-rate” describes the stability of the complex of the ABP on one hand and CTLA-4, preferably human CTLA-4 on the other hand, i.e. the fraction of complexes that decay per second. For example, a kd of 0.01 s-1 equates to 1% of the complexes decaying per second. In an embodiment, the dissociation rate constant (kd) is 1×10-3 s-1 or less, 1×10-4 s-1 or less, 1×10-5 s-1 or less, or 1×10-6 s-1 or less. The kd may be between 1×10-5 s-1 and 1×10-4 s-1; or between 1×10-4 s-1 and 1×10-3 s-1.

Competition between an anti-CTLA-4 ABP of a combination of the invention, or a method or use thereof, and a reference antibody, e.g. for binding CTLA-4, an epitope of CTLA-4, or a fragment of the CTLA-4, may be determined by competition ELISA, FMAT or Biacore. In one aspect, the competition assay is carried out by Biacore. There are several possible reasons for this competition: the two proteins may bind to the same or overlapping epitopes, there may be steric inhibition of binding, or binding of the first protein may induce a conformational change in the antigen that prevents or reduces binding of the second protein.

“Binding fragments” as used herein means a portion or fragment of the ABPs of a combination of the invention, or a method or use thereof, that include the antigen-binding site and are capable of binding CTLA-4 as defined herein, e.g. but not limited to capable of binding to the same epitope of the parent or full length antibody.

ABPs that bind human CTLA-4 are provided herein (i.e. an anti-CTLA-4 ABP, sometimes referred to herein as an “anti-CTLA-4 ABP” or an “anti-CTLA-4 antibody” and/or other variations of the same). These antibodies are useful in the treatment or prevention of acute or chronic diseases or conditions whose pathology involves CTLA-4 signalling. In one aspect, an antigen binding protein, or isolated human antibody or functional fragment of such protein or antibody, that binds to human CTLA-4 and is effective as a cancer treatment or treatment against disease is described, for example in combination with another compound such as an anti-OX40 ABP, suitably an agonist anti-OX40 ABP. Any of the antigen binding proteins or antibodies disclosed herein may be used as a medicament. Any one or more of the antigen binding proteins or antibodies may be used in the methods or compositions to treat cancer, e.g. those disclosed herein.

The isolated antibodies as described herein bind to human CTLA-4, and may bind to human CTLA-4, or genes or cDNA sequences having 90 percent homology or 90 percent identity thereto. The complete hCTLA-4 mRNA sequence can be found under GenBank Accession No. L15006. The protein sequence for human CTLA-4 can be found at GenBank Accession No. AAB59385.

Antigen binding proteins and antibodies that bind and/or modulate CTLA-4 are known in the art. Exemplary anti-CTLA-4 ABPs of a combination of the invention, or a method or use thereof, are disclosed, for example in U.S. Pat. Nos. 6,984,720; 7,605,238; 8,883,984; 8,491,895; 8,143,379; 7,411,057; 7,132,281; 7,109,003; 6,682,736, each of which is incorporated by reference in its entirety herein (To the extent any definitions conflict, this instant application controls).

In another embodiment, further embodiment, any mouse or chimeric sequences of any anti-CTLA-4 ABP of a combination of the invention, or a method or use thereof, are engineered to make a humanized antibody.

In another embodiment, the anti-CTLA-4 ABP of a combination of the invention, or a method or use thereof, comprises one or more (e.g. all) of the CDRs or VH or VL or HC (heavy chain) or LC (light chain) sequences of ipilimumab, or sequences with at least 90% (e.g., 90%, 91%, 92%, 93%, 94%, 95%, 96%, 97%, 98%, or 99%, 100%) sequence identity thereto.

The HC and LC CDRs of ipilimumab are provided below. In one embodiment, the anti-CTLA-4 ABP of a combination of the invention, or a method or use thereof, comprises: (a) a heavy chain variable region CDR1 of ipilimumab; (b) a heavy chain variable region CDR2 of ipilimumab; (c) a heavy chain variable region CDR3 of ipilimumab; (d) a light chain variable region CDR1 of ipilimumab; (e) a light chain variable region CDR2 of ipilimumab; and (f) a light chain variable region CDR3 of ipilimumab.

In another embodiment, the anti-CTLA-4 of a combination of the invention, or a method or use thereof, comprises: a heavy chain variable region CDR1 of ipilimumab; a heavy chain variable region CDR2 of ipilimumab and/or a heavy chain variable region CDR3 of ipilimumab.

In another embodiment, the anti-CTLA-4 of a combination of the invention, ora method or use thereof, comprises: a light chain variable region CDR1 of ipilimumab; a light chain variable region CDR2 of ipilimumab and/or a light chain variable region CDR3 of ipilimumab.

In another embodiment, the anti-CTLA-4 ABP of a combination of the invention, or a method or use thereof, comprises: a light chain variable region (“VL”) of ipilimumab, or an amino acid sequence with at least 90% (e.g., 90%, 91%, 92%, 93%, 94%, 95%, 96%, 97%, 98%, or 99%, 100%) sequence identity to the amino acid sequence of the VL of ipilimumab.

In another embodiment, the anti-CTLA-4 ABP of a combination of the invention, or a method or use thereof, comprises a heavy chain variable region (“VH”) of ipilimumab, or an amino acid sequence with at least 90% (e.g., 90%, 91%, 92%, 93%, 94%, 95%, 96%, 97%, 98%, or 99%, 100%) sequence identity to the amino acid sequence of the VH of ipilimumab.

In another embodiment, the anti-CTLA-4 ABP of a combination of the invention, or a method or use thereof, comprises: a light chain variable region (“VL”) of ipilimumab, or an amino acid sequence with at least 90% (e.g., 90%, 91%, 92%, 93%, 94%, 95%, 96%, 97%, 98%, or 99%, 100%) sequence identity to the amino acid sequence of the VL of ipilimumab and the anti-CTLA-4 ABP of a combination of the invention, or a method or use thereof, comprises a heavy chain variable region (“VH”) of ipilimumab, or an amino acid sequence with at least 90% (e.g., 90%, 91%, 92%, 93%, 94%, 95%, 96%, 97%, 98%, or 99%, 100%) sequence identity to the amino acid sequence of the VH of ipilimumab.

In another embodiment, the anti-CTLA-4 ABP of a combination of the invention, or a method or use thereof, comprises: a light chain (“LC”) of ipilimumab, or an amino acid sequence with at least 90% (e.g., 90%, 91%, 92%, 93%, 94%, 95%, 96%, 97%, 98%, or 99%, 100%) sequence identity to the amino acid sequence of the LC of ipilimumab.

In another embodiment, the anti-CTLA-4 ABP of a combination of the invention, or a method or use thereof, comprises a heavy chain (“HC”) of ipilimumab, or an amino acid sequence with at least 90% (e.g., 90%, 91%, 92%, 93%, 94%, 95%, 96%, 97%, 98%, or 99%, 100%) sequence identity to the amino acid sequence of the HC of ipilimumab.

In another embodiment, the anti-CTLA-4 ABP of a combination of the invention, or a method or use thereof, comprises: a light chain (“LC”) of ipilimumab, or an amino acid sequence with at least 90% (e.g., 90%, 91%, 92%, 93%, 94%, 95%, 96%, 97%, 98%, or 99%, 100%) sequence identity to the amino acid sequence of the LC of ipilimumab and the anti-CTLA-4 ABP of a combination of the invention, or a method or use thereof, comprises a heavy chain (“HC”) of ipilimumab, or an amino acid sequence with at least 90% (e.g., 90%, 91%, 92%, 93%, 94%, 95%, 96%, 97%, 98%, or 99%, 100%) sequence identity to the amino acid sequence of the HC of ipilimumab.

Another embodiment of a combination of the invention, or a method or use thereof, includes CDRs, VH regions, and VL regions, and antibodies and nucleic acids encoding the same as disclosed in the below sequences.

An anti-OX40 ABP (e.g., an agonist ABP, e.g. an anti-hOX40 ABP, e.g. antibody), e.g., an antibody described herein, can be used in combination with an ABP (e.g., antagonist ABP, e.g antagonist antibody) against CTLA-4 (e.g. human CTLA-4). For example, an anti-OX40 antibody can be used in combination with ipilimumab.

Ipilimumab (also known as, e.g., YERVOY®, BMS-734016) is disclosed, e.g., in U.S. Pat. Nos. 6,984,720 and 7,605,238. Ipilimumab is approved by the U.S. Food and Drug Administration (FDA) for the treatment of melanoma. It is undergoing clinical trials for the treatment of non-small cell lung carcinoma (NSCLC), small cell lung cancer (SCLC), bladder cancer and metastatic hormone-refractory prostate cancer.

The recommended dose of ipilimumab is 3 mg/kg administered intravenously over 90 minutes every 3 weeks for a total of 4 doses. Such a dose can be used, e.g., in a combination therapy with an OX40 antibody described herein.

Ipilimumab is a recombinant, human monoclonal antibody that binds to the cytotoxic T-lymphocyte-associated antigen 4 (CTLA-4). Ipilimumab is an IgG1 kappa immunoglobulin with an approximate molecular weight of 148 kDa. Ipilimumab is produced in mammalian (Chinese hamster ovary) cell culture. Ipilimumab is a sterile, preservative-free, clear to slightly opalescent, colorless to pale yellow solution for intravenous infusion, which may contain a small amount of visible translucent-to-white, amorphous ipilimumab particulates. It is supplied in single-use vials of 50 mg/10 mL and 200 mg/40 mL. Each milliliter contains 5 mg of ipilimumab and the following inactive ingredients: diethylene triamine pentaacetic acid (DTPA) (0.04 mg), mannitol (10 mg), polysorbate 80 (vegetable origin) (0.1 mg), sodium chloride (5.85 mg), tris hydrochloride (3.15 mg), and Water for Injection, USP at a pH of 7.

CTLA-4 is a negative regulator of T-cell activity. Ipilimumab is a monoclonal antibody that binds to CTLA-4 and blocks the interaction of CTLA-4 with its ligands, CD80/CD86. Blockade of CTLA-4 has been shown to augment T-cell activation and proliferation, including the activation and proliferation of tumor infiltrating T-effector cells. Inhibition of CTLA-4 signaling can also reduce T-regulatory cell function, which may contribute to a general increase in T cell responsiveness, including the anti-tumor immune response.

The heavy chain (HC) amino acid sequence of ipilimumab is:

(SEQ ID NO: 50)
QVQLVESGGG VVQPGRSLRL SCAASGFTFS SYTMHWVRQA
PGKGLEWVTF ISYDGNNKYY ADSVKGRFTI SRDNSKNTLY
LQMNSLRAED TAIYYCARTG WLGPFDYWGQ GTLVTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPELLGGPS
VFLFPPKPKD TLMISRTPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSRDELT
KNQVSLTCLV KGFYPSDIAV EWESNGQPEN NYKTTPPVLD
SDGSFFLYSK LTVDKSRWQQ GNVFSCSVMH EALHNHYTQK
SLSLSPGK

The light chain (LC) amino acid sequence of ipilimumab is:

(SEQ ID NO: 51)
EIVLTQSPGT LSLSPGERAT LSCRASQSVG SSYLAWYQQK
PGQAPRLLIY GAFSRATGIP DRFSGSGSGT DFTLTISRLE
PEDFAVYYCQ QYGSSPWTFG QGTKVEIKRT VAAPSVFIFP
PSDEQLKSGT ASVVCLLNNF YPREAKVQWK VDNALQSGNS
QESVTEQDSK DSTYSLSSTL TLSKADYEKH KVYACEVTHQ
GLSSPVTKSF NRGEC

The heavy chain variable region (VH) amino acid sequence of ipilimumab is:

```
(SEQ ID NO: 52)
Gln Val Gln Leu Val Glu Ser Gly Gly Gly Val Val
  1 5 10
Gln Pro Gly Arg Ser Leu Arg Leu Ser Cys Ala Ala
                  15 20
Ser Gly Phe Thr Phe Ser Ser Tyr Thr Met His Trp
  25 30 35
Val Arg Gln Ala Pro Gly Lys Gly Leu Glu Trp Val
                          40 45
Thr Phe Ile Ser Tyr Asp Gly Asn Asn Lys Tyr Tyr
          50 55 60
Ala Asp Ser Val Lys Gly Arg Phe Thr Ile Ser Arg
                                  65 70
Asp Asn Ser Lys Asn Thr Leu Tyr Leu Gln Met Asn
                  75 80
Ser Leu Arg Ala Glu Asp Thr Ala Ile Tyr Tyr Cys
  85 90 95
Ala Arg Thr Gly Trp Leu Gly Pro Phe Asp Tyr Trp
                        100 105
Gly Gln Gly Thr Leu Val Thr Val Ser Ser
        110
```

The light chain variable region (VL) amino acid sequence of ipilimumab is:

```
(SEQ ID NO: 53)
Glu Ile Val Leu Thr Gln Ser Pro Gly Thr Leu Ser
  1 5 10
Leu Ser Pro Gly Glu Arg Ala Thr Leu Ser Cys Arg
                  15 20
Ala Ser Gln Ser Val Gly Ser Ser Tyr Leu Ala Trp
  25 30 35
Tyr Gln Gln Lys Pro Gly Gln Ala Pro Arg Leu Leu
                          40 45
Ile Tyr Gly Ala Phe Ser Arg Ala Thr Gly Ile Pro
          50 55 60
Asp Arg Phe Ser Gly Ser Gly Ser Gly Thr Asp Phe
                                  65 70
Thr Leu Thr Ile Ser Arg Leu Glu Pro Glu Asp Phe
                  75 80
Ala Val Tyr Tyr Cys Gln Gln Tyr Gly Ser Ser Pro
  85 90 95
Trp Thr Phe Gly Gln Gly Thr Lys Val Glu Ile Lys
```

The CDR sequences of ipilimumab are:

HC CDR1:
(SEQ ID NO: 54)
SYTMH
HC CDR2:
(SEQ ID NO: 55)
FISYDGNNKYYADSVKG
HC CDR3:
(SEQ ID NO: 56)
TGWLGPFDY
LC CDR1:
(SEQ ID NO: 57)
RASQSVGSSYLA
LC CDR2:
(SEQ ID NO: 58)
GAFSRAT
LC CDR3:
(SEQ ID NO: 59)
QQYGSSPWT

Ipilimumab, or an antibody that comprises the same CDR sequences as ipilimumab, can be used in combination with an OX40 antibody described herein.

As another example, an anti-OX40 antibody can be used in combination with tremelimumab.

Tremelimumab (formerly ticilimumab, CP-675,206) is a fully human IgG2 monoclonal antibody. See, e.g., U.S. Pat. Nos. 8,883,984; 8,491,895; 8,143,379; 7,411,057; 7,132,281; 7,109,003; 6,682,736; U.S. App. Pub. No. 2008-0279865.

In another embodiment, the anti-CTLA-4 ABP of a combination of the invention, or a method or use thereof, comprises one or more (e.g. all) of the CDRs or VH or VL or HC (heavy chain) or LC (light chain) sequences of tremelimumab, or sequences with at least 90% (e.g., 90%, 91%, 92%, 93%, 94%, 95%, 96%, 97%, 98%, or 99%, 100%) sequence identity thereto.

The HC and LC CDRs of tremelimumab are provided herein. In one embodiment, the anti-CTLA-4 ABP of a combination of the invention, or a method or use thereof, comprises: (a) a heavy chain variable region CDR1 of tremelimumab; (b) a heavy chain variable region CDR2 of tremelimumab; (c) a heavy chain variable region CDR3 of tremelimumab; (d) a light chain variable region CDR1 of tremelimumab; (e) a light chain variable region CDR2 of tremelimumab; and (f) a light chain variable region CDR3 of tremelimumab.

In another embodiment, the anti-CTLA-4 ABP of a combination of the invention, or a method or use thereof, comprises: a heavy chain variable region CDR1 of tremelimumab; a heavy chain variable region CDR2 of tremelimumab and/or a heavy chain variable region CDR3 of tremelimumab.

In another embodiment, the anti-CTLA-4 ABP of a combination of the invention, or a method or use thereof, comprises: a light chain variable region CDR1 of tremelimumab; a light chain variable region CDR2 of tremelimumab and/or a light chain variable region CDR3 of tremelimumab.

In another embodiment, the anti-CTLA-4 ABP of a combination of the invention, or a method or use thereof, comprises: a light chain variable region (“VL”) of tremelimumab, or an amino acid sequence with at least 90% (e.g., 90%, 91%, 92%, 93%, 94%, 95%, 96%, 97%, 98%, or 99%, 100%) sequence identity to the amino acid sequence of the VL of tremelimumab.

In another embodiment, the anti-CTLA-4 ABP of a combination of the invention, or a method or use thereof, comprises a heavy chain variable region (“VH”) of tremelimumab, or an amino acid sequence with at least 90% (e.g., 90%, 91%, 92%, 93%, 94%, 95%, 96%, 97%, 98%, or 99%, 100%) sequence identity to the amino acid sequence of the VH of tremelimumab.

In another embodiment, the anti-CTLA-4 ABP of a combination of the invention, or a method or use thereof, comprises: a light chain variable region (“VL”) of tremelimumab, or an amino acid sequence with at least 90% (e.g., 90%, 91%, 92%, 93%, 94%, 95%, 96%, 97%, 98%, or 99%, 100%) sequence identity to the amino acid sequence of the VL of tremelimumab and the anti-CTLA-4 ABP of a combination of the invention, or a method or use thereof, comprises a heavy chain variable region (“VH”) of tremelimumab, or an amino acid sequence with at least 90% (e.g., 90%, 91%, 92%, 93%, 94%, 95%, 96%, 97%, 98%, or 99%, 100%) sequence identity to the amino acid sequence of the VH of tremelimumab.

In another embodiment, the anti-CTLA-4 ABP of a combination of the invention, or a method or use thereof, comprises: a light chain (“LC”) of tremelimumab, or an amino acid sequence with at least 90% (e.g., 90%, 91%, 92%, 93%, 94%, 95%, 96%, 97%, 98%, or 99%, 100%) sequence identity to the amino acid sequence of the LC of tremelimumab.

In another embodiment, the anti-CTLA-4 ABP of a combination of the invention, or a method or use thereof, comprises a heavy chain (“HC”) of tremelimumab, or an amino acid sequence with at least 90% (e.g., 90%, 91%, 92%, 93%, 94%, 95%, 96%, 97%, 98%, or 99%, 100%) sequence identity to the amino acid sequence of the HC of tremelimumab.

In another embodiment, the anti-CTLA-4 ABP of a combination of the invention, or a method or use thereof, comprises: a light chain (“LC”) of tremelimumab, or an amino acid sequence with at least 90% (e.g., 90%, 91%, 92%, 93%, 94%, 95%, 96%, 97%, 98%, or 99%, 100%) sequence identity to the amino acid sequence of the LC of tremelimumab and the anti-CTLA-4 ABP of a combination of the invention, or a method or use thereof, comprises a heavy chain (“HC”) of tremelimumab, or an amino acid sequence with at least 90% (e.g., 90%, 91%, 92%, 93%, 94%, 95%, 96%, 97%, 98%, or 99%, 100%) sequence identity to the amino acid sequence of the HC oftremelimumab.

Another embodiment of a combination of the invention, or a method or use thereof, includes CDRs, VH regions, and VL regions, HC, and LC, and antibodies and nucleic acids encoding the same as disclosed in the below sequences.

An anti-OX40 ABP (e.g., an agonist ABP, e.g. an anti-hOX40 ABP, e.g. antibody), e.g., an antibody described herein, can be used in combination with an ABP (e.g., antagonist ABP, e.g antagonist antibody) against CTLA-4 (e.g. human CTLA-4). For example, an anti-OX40 antibody can be used in combination with tremelimumab.

The heavy chain (HC) amino acid sequence of tremelimumab is:

```
(SEQ ID NO: 60)
Gln Val Gln Leu Val Glu Ser Gly Gly Gly Val Val Gln Pro Gly Arg
1 5 10 15
Ser Leu Arg Leu Ser Cys Ala Ala Ser Gly Phe Thr Phe Ser Ser Tyr
                        20 25 30
Gly Met His Trp Val Arg Gln Ala Pro Gly Lys Gly Leu Glu Trp Val
                35 40 45
Ala Val Ile Trp Tyr Asp Gly Ser Asn Lys Tyr Tyr Ala Asp Ser Val
        50 55 60
Lys Gly Arg Phe Thr Ile Ser Arg Asp Asn Ser Lys Asn Thr Leu Tyr
65 70 75 80
Leu Gln Met Asn Ser Leu Arg Ala Glu Asp Thr Ala Val Tyr Tyr Cys
                                85 90 95
Ala Arg Asp Pro Arg Gly Ala Thr Leu Tyr Tyr Tyr Tyr Tyr Gly Met
                        100 105 110
Asp Val Trp Gly Gln Gly Thr Thr Val Thr Val Ser Ser Ala Ser Thr
                115 120 125
Lys Gly Pro Ser Val Phe Pro Leu Ala Pro Cys Ser Arg Ser Thr Ser
        130 135 140
Glu Ser Thr Ala Ala Leu Gly Cys Leu Val Lys Asp Tyr Phe Pro Glu
145 150 155 160
Pro Val Thr Val Ser Trp Asn Ser Gly Ala Leu Thr Ser Gly Val His
                                165 170 175
Thr Phe Pro Ala Val Leu Gln Ser Ser Gly Leu Tyr Ser Leu Ser Ser
                        180 185 190
Val Val Thr Val Pro Ser Ser Asn Phe Gly Thr Gln Thr Tyr Thr Cys
                195 200 205
Asn Val Asp His Lys Pro Ser Asn Thr Lys Val Asp Lys Thr Val Glu
        210 215 220
Arg Lys Cys Cys Val Glu Cys Pro Pro Cys Pro Ala Pro Pro Val Ala
225 230 235 240
Gly Pro Ser Val Phe Leu Phe Pro Pro Lys Pro Lys Asp Thr Leu Met
                                245 250 255
Ile Ser Arg Thr Pro Glu Val Thr Cys Val Val Val Asp Val Ser His
                        260 265 270
Glu Asp Pro Glu Val Gln Phe Asn Trp Tyr Val Asp Gly Val Glu Val
                275 280 285
His Asn Ala Lys Thr Lys Pro Arg Glu Glu Gln Phe Asn Ser Thr Phe
        290 295 300
Arg Val Val Ser Val Leu Thr Val Val His Gln Asp Trp Leu Asn Gly
305 310 315 320
Lys Glu Tyr Lys Cys Lys Val Ser Asn Lys Gly Leu Pro Ala Pro Ile
                                325 330 335
Glu Lys Thr Ile Ser Lys Thr Lys Gly Gln Pro Arg Glu Pro Gln Val
                        340 345 350
Tyr Thr Leu Pro Pro Ser Arg Glu Glu Met Thr Lys Asn Gln Val Ser
                355 360 365
Leu Thr Cys Leu Val Lys Gly Phe Tyr Pro Ser Asp Ile Ala Val Glu
        370 375 380
Trp Glu Ser Asn Gly Gln Pro Glu Asn Asn Tyr Lys Thr Thr Pro Pro
385 390 395 400
Met Leu Asp Ser Asp Gly Ser Phe Phe Leu Tyr Ser Lys Leu Thr Val
                                405 410 415
Asp Lys Ser Arg Trp Gln Gln Gly Asn Val Phe Ser Cys Ser Val Met
                        420 425 430
His Glu Ala Leu His Asn His Tyr Thr Gln Lys Ser Leu Ser Leu Ser
                435 440 445
Pro Gly Lys
        450
```

The light chain (LC) amino acid sequence of tremelimumab is:

```
(SEQ ID NO: 61)
Asp Ile Gln Met Thr Gln Ser Pro Ser Ser Leu Ser
1 5 10
Ala Ser Val Gly Asp Arg Val Thr Ile Thr Cys Arg
                15 20
Ala Ser Gln Ser Ile Asn Ser Tyr Leu Asp Trp Tyr
25 30 35
Gln Gln Lys Pro Gly Lys Ala Pro Lys Leu Leu Ile
                        40 45
Tyr Ala Ala Ser Ser Leu Gln Ser Gly Val Pro Ser
        50 55 60
Arg Phe Ser Gly Ser Gly Ser Gly Thr Asp Phe Thr
                                65 70
Leu Thr Ile Ser Ser Leu Gln Pro Glu Asp Phe Ala
                75 80
Thr Tyr Tyr Cys Gln Gln Tyr Tyr Ser Thr Pro Phe
85 90 95
Thr Phe Gly Pro Gly Thr Lys Val Glu Ile Lys Arg
                        100 105
Thr Val Ala Ala Pro Ser Val Phe Ile Phe Pro Pro
        110 115 120
Ser Asp Glu Gln Leu Lys Ser Gly Thr Ala Ser Val
                                125 130
Val Cys Leu Leu Asn Asn Phe Tyr Pro Arg Glu Ala
                135 140
Lys Val Gln Trp Lys Val Asp Asn Ala Leu Gln Ser
145 150 155
Gly Asn Ser Gln Glu Ser Val Thr Glu Gln Asp Ser
                        160 165
Lys Asp Ser Thr Tyr Ser Leu Ser Ser Thr Leu Thr
        170 175 180
Leu Ser Lys Ala Asp Tyr Glu Lys His Lys Val Tyr
                                185 190
Ala Cys Glu Val Thr His Gln Gly Leu Ser Ser Pro
                195 200
Val Thr Lys Ser Phe Asn Arg Gly Glu Cys
205 210
```

The heavy chain variable region (VH) amino acid sequence of tremelimumab is:

```
(SEQ ID NO: 62)
Gln Val Gln Leu Val Glu Ser Gly Gly Gly Val Val
1 5 10
Gln Pro Gly Arg Ser Leu Arg Leu Ser Cys Ala Ala
                15 20
Ser Gly Phe Thr Phe Ser Ser Tyr Gly Met His Trp
25 30 35
Val Arg Gln Ala Pro Gly Lys Gly Leu Glu Trp Val
                        40 45
Ala Val Ile Trp Tyr Asp Gly Ser Asn Lys Tyr Tyr
        50 55 60
Ala Asp Ser Val Lys Gly Arg Phe Thr Ile Ser Arg
                                65 70
Asp Asn Ser Lys Asn Thr Leu Tyr Leu Gln Met Asn
                75 80
Ser Leu Arg Ala Glu Asp Thr Ala Val Tyr Tyr Cys
85 90 95
Ala Arg Asp Pro Arg Gly Ala Thr Leu Tyr Tyr Tyr
                        100 105
Tyr Tyr Gly Met Asp Val Trp Gly Gln Gly Thr Thr
        110 115 120
Val Thr Val Ser Ser
                                125
```

The light chain variable region (VL) amino acid sequence of tremelimumab is:

```
(SEQ ID NO: 63)
Asp Ile Gln Met Thr Gln Ser Pro Ser Ser Leu Ser
1 5 10
Ala Ser Val Gly Asp Arg Val Thr Ile Thr Cys Arg
                15 20
Ala Ser Gln Ser Ile Asn Ser Tyr Leu Asp Trp Tyr
25 30 35
Gln Gln Lys Pro Gly Lys Ala Pro Lys Leu Leu Ile
                        40 45
Tyr Ala Ala Ser Ser Leu Gln Ser Gly Val Pro Ser
        50 55 60
Arg Phe Ser Gly Ser Gly Ser Gly Thr Asp Phe Thr
                                65 70
Leu Thr Ile Ser Ser Leu Gln Pro Glu Asp Phe Ala
                75 80
Thr Tyr Tyr Cys Gln Gln Tyr Tyr Ser Thr Pro Phe
85 90 95
Thr Phe Gly Pro Gly Thr Lys Val Glu Ile Lys
                        100 105
```

The CDR sequences of tremelimumab are:

HC CDR1:
(SEQ ID NO: 64)
GFTFSSYGMH
HC CDR2:
(SEQ ID NO: 65)
VIWYDGSNKYYADSV
HC CDR3:
(SEQ ID NO: 66)
DPRGATLYYYYYGMDV
LC CDR1:
(SEQ ID NO: 67)
RASQSINSYLD
LC CDR2:
(SEQ ID NO: 68)
AASSLQS
LC CDR3:
(SEQ ID NO: 69)
QQYYSTPFT

As additional examples, anti-OX40 ABP (e.g., an agonist ABP, e.g. an anti-hOX40 ABP, e.g. antibody), e.g., an antibody described herein, can be used in combination with a CTLA-4 antibody, e.g., that binds to human CTLA-4 so as to inhibit CTLA-4 from interacting with a human B7 counterreceptor. Because interaction of human CTLA-4 with human B7 transduces a signal leading to inactivation of T-cells bearing the human CTLA-4 receptor, antagonism of the interaction effectively induces, augments or prolongs the activation of T-cells bearing the human CTLA-4 receptor, thereby prolonging or augmenting an immune response.

Anti-CTLA-4 antibodies are described in, e.g., U.S. Pat. Nos. 9,084,776; 5,811,097; 5,855,887; 6,051,227; 6,984,720; 7,605,238; 8,883,984; in PCT Application Publication No. WO 01/14424 and WO 00/37504; and in U.S. App. Pub. Nos. 2002-0039581; 2015-0104409; 2008-0279865. An exemplary clinical anti-CTLA-4 antibody is human monoclonal antibody 10D1 as disclosed in WO 01/14424 and U.S. Pat. No. 6,984,720. Antibody 10D1 has been administered in single and multiple doses, alone or combination with a vaccine, chemotherapy, or interleukin-2 to more than 500 patients diagnosed with metastatic melanoma, prostate cancer, lymphoma, renal cell cancer, breast cancer, ovarian cancer, and HIV. Any of these CTLA-4 antibodies can be used in combination with an anti-OX40 ABP (e.g. antibody) described herein.

Other anti-CTLA-4 antibodies also encompassed by the methods of the present invention include, for example those disclosed in WO 98/42752; WO 00/37504; U.S. Pat. No. 6,207,156; Hurwitz el al. (1998) Proc. Natl. Acad. Sci. U.S.A. 95(17):10067-10071; Camacho et. al. (2004) J. Clin. Oncology 22(145): Abstract No 2505 (antibody CP-675206); and Mokyr et. al. (1998) Cancer Res. 58:5301-5304. Any of these CTLA-4 antibodies can be used in combination with an anti-OX40 ABP (e.g. antibody) described herein.

In one embodiment, the present invention provides methods of treating cancer in a mammal in need thereof comprising administering a therapeutically effective amount of an antigen binding protein that binds OX40 and an antigen binding protein that binds CTLA-4. In some aspects the cancer is a solid tumor. The cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer. In another aspect the cancer is a liquid tumor.

In one embodiment, the antigen binding protein that binds OX40 and the antigen binding that binds CTLA-4 are administered at the same time. In another embodiment, antigen binding protein that binds OX40 and the antigen binding protein that binds CTLA-4 are administered sequentially, in any order. In one aspect, the antigen binding protein that binds OX40 and/or the antigen binding protein that binds CTLA-4 are administered systemically, e.g. intravenously. In another aspect, the antigen binding protein that binds OX40 and/or the antigen binding protein that binds CTLA-4 is administered intratumorally.

In one embodiment, the mammal is human.

Methods are provided wherein the tumor size of the cancer in said mammal is reduced by more than an additive amount compared with treatment with the antigen binding protein to OX-40 and the antigen binding protein to CTLA-4 as used as monotherapy. Suitably the combination may be synergistic.

In one embodiment, the antigen binding protein that binds OX40 binds to human OX40. In one embodiment, the antigen binding protein that binds to CTLA-4 binds to human CTLA-4. In one embodiment, the antigen binding protein that binds OX40 and/or the antigen binding protein that binds CTLA-4 is a humanized monoclonal antibody. In one embodiment, the antigen binding protein that binds OX40 and/or the antigen binding protein that binds CTLA-4 is a fully human monoclonal antibody.

The antigen binding protein that binds OX40 is an antibody with an IgG1 isotype or variant thereof. In one embodiment, the antigen binding protein that binds CTLA-4 is an antibody with an IgG1 isotype or variant thereof. The antigen binding protein that binds OX40 is an antibody with an IgG4 isotype or variant thereof. In one embodiment, the antigen binding protein that binds CTLA-4 is an antibody with an IgG4 isotype or variant thereof. In one aspect the antigen binding protein that binds OX40 is an agonist antibody. In one aspect the antigen binding protein that binds CTLA-4 is an antagonist antibody.

Suitably, the antigen binding protein that binds OX40 comprises: a heavy chain variable region CDR1 comprising an amino acid sequence with at least 90% 91%, 92%, 93%, 94%, 95%, 96%, 97%, 98%, 99% or 100% sequence identity to the amino acid sequence set forth in SEQ ID NO: 1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO: 2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO: 3 or 15.

Suitably, the antigen binding protein that binds OX40 comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO: 7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO: 8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO: 9 or 21.

Suitably, the antigen binding protein that binds OX40 comprises: (a) a heavy chain variable region CDR1 comprising the amino acid sequence of SEQ ID NO: 1; (b) a heavy chain variable region CDR2 comprising the amino acid sequence of SEQ ID NO: 2; (c) a heavy chain variable region CDR3 comprising the amino acid sequence of SEQ ID NO. 3; (d) a light chain variable region CDR1 comprising the amino acid sequence of SEQ ID NO. 7; (e) a light chain variable region CDR2 comprising the amino acid sequence of SEQ ID NO. 8; and (f) a light chain variable region CDR3 comprising the amino acid sequence of SEQ ID NO. 9.

Suitably, the antigen binding protein that binds OX40 comprises: (a) a heavy chain variable region CDR1 comprising the amino acid sequence of SEQ ID NO: 13; (b) a heavy chain variable region CDR2 comprising the amino acid sequence of SEQ ID NO: 14; (c) a heavy chain variable region CDR3 comprising the amino acid sequence of SEQ ID NO. 15; (d) a light chain variable region CDR1 comprising the amino acid sequence of SEQ ID NO. 19; (e) a light chain variable region CDR2 comprising the amino acid sequence of SEQ ID NO. 20; and (f) a light chain variable region CDR3 comprising the amino acid sequence of SEQ ID NO. 21.

Suitably, the antigen binding protein that binds OX40 comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO: 10, 11, 22 or 23. Suitably, the antigen binding protein that binds OX40 comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO: 4, 5, 16 and 17. Suitably, the antigen binding protein that binds OX40 comprises a heavy chain variable region comprising the amino acid sequence set forth in SEQ ID NO:5 and a light chain variable region comprising the amino acid sequence set forth in SEQ ID NO: 11.

Suitably, the antigen binding protein that binds OX40 comprises a heavy chain variable region comprising the amino acid sequence set forth in SEQ ID NO:17 and a light chain variable region comprising the amino acid sequence set forth in SEQ ID NO: 23. Suitably, the antigen binding protein that binds OX40 comprises a light chain variable region comprising the amino acid sequence of SEQ ID NO: 11 or 23, or an amino acid sequence with at least 90% sequence identity to the amino acid sequences of SEQ ID NO: 11 or 23. Suitably, the antigen binding protein that binds OX40 comprises a heavy chain variable region comprising the amino acid sequence of SEQ ID NO: 5 or 17, or an amino acid sequence with at least 90% sequence identity to the amino acid sequences of SEQ ID NO: 5 or 17.

In one embodiment, the antigen binding protein that binds CTLA-4 is ipilimumab, or an antibody comprising 90%, 91%, 92%, 93%, 94%, 95%, 96%, 97%, 98%, 99% or 100% sequence identity thereto. In another embodiment, the antigen binding protein that binds CTLA-4 is tremelimumab, or an antibody having 90%, 91%, 92%, 93%, 94%, 95%, 96%, 97%, 98%, 99% or 100% sequence identity thereto.

In one aspect, the mammal has increased survival when treated with a therapeutically effective amount of an antigen binding protein to OX-40 and therapeutically effective amount of an antigen binding protein to CTLA-4 compared with a mammal who received the antigen binding protein to OX-40 or the antigen binding protein to CTLA-4 as monotherapy. In one aspect, the methods further comprise administering at least one anti-neoplastic agent to the mammal in need thereof.

In one embodiment, pharmaceutical compositions are provided comprising a therapeutically effective amount of an antigen binding protein that binds OX40 and a therapeutically effective amount of an antigen binding protein that binds CTLA-4.

In one embodiment, the pharmaceutical compositions comprise an antibody comprising an antigen binding protein that binds OX40 comprising a CDRH1 having an amino acid sequence with at least 90%, 91%, 92%, 93%, 94%, 95%, 96%, 97%, 98%, 99% or 100% sequence identity to the amino acid sequence as set forth in SEQ ID NO: 1, a CDRH2 having an amino acid sequence with at least 90%, 91%, 92%, 93%, 94%, 95%, 96%, 97%, 98%, 99% or 100% sequence identity to the amino acid sequence as set forth in SEQ ID NO: 2, a CDRH3 having an amino acid sequence with at least 90%, 91%, 92%, 93%, 94%, 95%, 96%, 97%, 98%, 99% or 100% sequence identity to the amino acid sequence as set forth in SEQ ID NO: 3, a CDRL1 having an amino acid sequence with at least 90%, 91%, 92%, 93%, 94%, 95%, 96%, 97%, 98%, 99% or 100% sequence identity to the amino acid sequence as set forth in SEQ ID NO: 7, a CDRL2 having an amino acid sequence with at least 90%, 91%, 92%, 93%, 94%, 95%, 96%, 97%, 98%, 99% or 100% sequence identity to the amino acid sequence as set forth in SEQ ID NO: 8, a CDRL3 having an amino acid sequence with at least 90%, 91%, 92%, 93%, 94%, 95%, 96%, 97%, 98%, 99% or 100% sequence identity to the amino acid sequence as set forth in SEQ ID NO: 9; and ipilimumab, or an antibody comprising 90%, 91%, 92%, 93%, 94%, 95%, 96%, 97%, 98%, 99% or 100% sequence identity thereto.

In one embodiment, the pharmaceutical compositions of the present invention comprise an antibody comprising a VH region having a sequence at least with a sequence at least 90%, 91%, 92%, 93%, 94%, 95%, 96%, 97%, 98%, 99% or 100% sequence identity to the amino acid sequence as set forth in SEQ ID NO: 4 or 5 and VL having a sequence at least with a sequence at least 90%, 91%, 92%, 93%, 94%, 95%, 96%, 97%, 98%, 99% or 100% sequence identity to the amino acid sequence as set forth in SEQ ID NO:10 or 11, and pembrolizumab, or an antibody comprising 90%, 91%, 92%, 93%, 94%, 95%, 96%, 97%, 98%, 99% or 100% sequence identity thereto.

In one embodiment, the pharmaceutical compositions of the present invention comprise an antibody comprising an antigen binding protein that binds OX40 comprising a CDRH1 having an amino acid sequence with at least 90%, 91%, 92%, 93%, 94%, 95%, 96%, 97%, 98%, 99% or 100% sequence identity to the amino acid sequence as set forth in SEQ ID NO: 1, a CDRH2 having an amino acid sequence with at least 90%, 91%, 92%, 93%, 94%, 95%, 96%, 97%, 98%, 99% or 100% sequence identity to the amino acid sequence as set forth in SEQ ID NO: 2, a CDRH3 having an amino acid sequence with at least 90%, 91%, 92%, 93%, 94%, 95%, 96%, 97%, 98%, 99% or 100% sequence identity to the amino acid sequence as set forth in SEQ ID NO: 3, a CDRL1 having an amino acid sequence with at least 90%, 91%, 92%, 93%, 94%, 95%, 96%, 97%, 98%, 99% or 100% sequence identity to the amino acid sequence as set forth in SEQ ID NO: 7, a CDRL2 having an amino acid sequence with at least 90%, 91%, 92%, 93%, 94%, 95%, 96%, 97%, 98%, 99% or 100% sequence identity to the amino acid sequence as set forth in SEQ ID NO: 8, a CDRL3 having an amino acid sequence with at least 90%, 91%, 92%, 93%, 94%, 95%, 96%, 97%, 98%, 99% or 100% sequence identity to the amino acid sequence as set forth in SEQ ID NO: 9; and tremelimumab, or an antibody comprising 90%, 91%, 92%, 93%, 94%, 95%, 96%, 97%, 98%, 99% or 100% sequence identity thereto.

Also provided in the present invention are the use of a combination or pharmaceutical compositions of this invention in the manufacture of a medicament for the treatment of cancer. Also provided are the use of pharmaceutical compositions of the present invention for treating cancer. The present invention also provides combination kit comprising pharmaceutical compositions of the invention together with one or more pharmaceutically acceptable carriers.

In one embodiment methods are provided for reducing tumor size in a human having cancer comprising administering a therapeutically effective amount of an agonist antibody to human OX-40 and a therapeutically effective amount of an antagonist antibody to human CTLA-4.

### Methods of Treatment

The combinations of the invention are believed to have utility in disorders wherein the engagement of OX40 (e.g., agonistic engagement) and/or CTLA-4 (e.g., antagonistic engagement), is beneficial.

The present invention thus also provides a combination of the invention, for use in therapy, particularly in the treatment of disorders wherein the engagement of OX40 (e.g., agonistic engagement) and/or CTLA-4 (e.g., antagonistic engagement), is beneficial, particularly cancer.

A further aspect of the invention provides a method of treatment of a disorder wherein engagement of OX40 (e.g., agonistic engagement) and/or CTLA-4 (e.g., antagonistic engagement), comprising administering a combination of the invention.

A further aspect of the present invention provides the use of a combination of the invention in the manufacture of a medicament for the treatment of a disorder engagement of OX40 (e.g., agonistic engagement) and/or CTLA-4 (e.g., antagonistic engagement), is beneficial. In preferred embodiments the disorder is cancer.

Examples of cancers that are suitable for treatment with combination of the invention include, but are limited to, both primary and metastatic forms of head and neck, breast, lung, colon, ovary, and prostate cancers. Suitably the cancer is selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Additionally, examples of a cancer to be treated include Barret's adenocarcinoma; billiary tract carcinomas; breast cancer; cervical cancer; cholangiocarcinoma; central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system); colorectal cancer including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; skin cancers including melanomas; and thyroid cancers.

Suitably, the present invention relates to a method for treating or lessening the severity of a cancer selected from: brain (gliomas), glioblastomas, astrocytomas, glioblastoma multiforme, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, breast, pancreatic and prostate.

Suitably, the present invention relates to a method for treating or lessening the severity of non-small cell lung carcinoma (NSCLC), small cell lung cancer (SCLC), bladder cancer and metastatic hormone-refractory prostate cancer.

Suitably, the present invention relates to a method for treating or lessening the severity of melanoma, e.g. metastatic melanoma.

Suitably the present invention relates to a method for treating or lessening the severity of pre-cancerous syndromes in a mammal, including a human, wherein the pre-cancerous syndrome is selected from: cervical intraepithelial neoplasia, monoclonal gammapathy of unknown significance (MGUS), myelodysplastic syndrome, aplastic anemia, cervical lesions, skin nevi (pre-melanoma), prostatic intraepithleial (intraductal) neoplasia (PIN), Ductal Carcinoma in situ (DCIS), colon polyps and severe hepatitis or cirrhosis.

The combination of the invention may be used alone or in combination with one or more other therapeutic agents. The invention thus provides in a further aspect a further combination comprising a combination of the invention with a further therapeutic agent or agents, compositions and medicaments comprising the combination and use of the further combination, compositions and medicaments in therapy, in particular in the treatment of diseases susceptible engagement of OX40, e.g. agonism of OX40, and/or CTLA-4, e.g. antagonism of CTLA-4.

In the embodiment, the combination of the invention may be employed with other therapeutic methods of cancer treatment. In particular, in anti-neoplastic therapy, combination therapy with other chemotherapeutic, hormonal, antibody agents as well as surgical and/or radiation treatments other than those mentioned above are envisaged. Combination therapies according to the present invention thus include the administration of an anti-OX40 ABP of a combination, or method or use thereof, of the invention and/or an anti-CTLA-4 ABP of a combination, or method or use thereof, of the invention as well as optional use of other therapeutic agents including other anti-neoplastic agents. Such combination of agents may be administered together or separately and, when administered separately this may occur simultaneously or sequentially in any order, both close and remote in time. In one embodiment, the pharmaceutical combination includes an anti-OX40 ABP, suitably an agonist anti-OX40 ABP and an anti-CTLA-4 ABP, suitably an antagonist anti-CTLA-4 ABP, and optionally at least one additional anti-neoplastic agent.

In one embodiment, the further anti-cancer therapy is surgical and/or radiotherapy.

In one embodiment, the further anti-cancer therapy is at least one additional anti-neoplastic agent.

Any anti-neoplastic agent that has activity versus a susceptible tumor being treated may be utilized in the combination. Typical anti-neoplastic agents useful include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine angiogenesis inhibitors; immunotherapeutic agents; proapoptotic agents; and cell cycle signaling inhibitors.

Anti-microtubule or anti-mitotic agents: Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 513,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. Paclitaxel has been approved for clinical use in the treatment of refractory ovarian cancer in the United States (Markman et al., Yale Journal of Biology and Medicine, 64:583, 1991; McGuire et al., Ann. Intern, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,βα-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin Ill, extracted from the needle of the European Yew tree.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes: Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, oxaliplatin, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma.

Alkylating agents: Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease.

Busulfan, 1,4-butanediol dimethanesulfonate, is commercially available as MYLERAN® TABLETS. Busulfan is indicated for the palliative treatment of chronic myelogenous leukemia.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease.

Antibiotic anti-neoplastics: Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma.

Doxorubicin, (8S, 10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas.

Topoisomerase II inhibitors: Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children.

Antimetabolite neoplastic agents: Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine).

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Other purine analogs include pentostatin, erythrohydroxynonyladenine, fludarabine phosphate, and cladribine.

Gemcitabine, 2′-deoxy-2′, 2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder.

Topoisomerase I inhibitors: Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®. Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer.

Hormones and hormonal analogues: Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors: Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, ret, vascular endothelial growth factor receptor (VEGFr), tyrosine kinase with immunoglobulin-like and epidermal growth factor identity domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-lacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kinases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Anti-angiogenic agents: Anti-angiogenic agents including non-receptorMEKngiogenesis inhibitors may alo be useful. Anti-angiogenic agents such as those which inhibit the effects of vascular edothelial growth factor, (for example the anti-vascular endothelial cell growth factor antibody bevacizumab [Avastin™], and compounds that work by other mechanisms (for example linomide, inhibitors of integrin αvβ3 function, endostatin and angiostatin);

Immunotherapeutic agents: Agents used in immunotherapeutic regimens may also be useful in combination with the compounds of formula (I). Immunotherapy approaches, including for example ex-vivo and in-vivo approaches to increase the immunogenecity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies

Proapoptotoc agents: Agents used in proapoptotic regimens (e.g., bcl-2 antisense oligonucleotides) may also be used in the combination of the present invention.

Cell cycle signalling inhibitors: Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the combination of the present invention comprises an anti-OX40 ABP and a CTLA-4 modulator (e.g. anti-CTLA-4 ABP) and at least one anti-neoplastic agent selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine MEKngiogenesis inhibitors, immunotherapeutic agents, proapoptotic agents, and cell cycle signaling inhibitors.

In one embodiment, the combination of the present invention comprises an anti-OX40 ABP and a CTLA-4 modulator (e.g. anti-CTLA-4 ABP) and at least one anti-neoplastic agent which is an anti-microtubule agent selected from diterpenoids and vinca alkaloids.

In a further embodiment, the at least one anti-neoplastic agent agent is a diterpenoid.

In a further embodiment, the at least one anti-neoplastic agent is a vinca alkaloid.

In one embodiment, the combination of the present invention comprises an anti-OX40 ABP and a CTLA-4 modulator (e.g. anti-CTLA-4 ABP) and at least one anti-neoplastic agent, which is a platinum coordination complex.

In a further embodiment, the at least one anti-neoplastic agent is paclitaxel, carboplatin, or vinorelbine.

In a further embodiment, the at least one anti-neoplastic agent is carboplatin.

In a further embodiment, the at least one anti-neoplastic agent is vinorelbine.

In a further embodiment, the at least one anti-neoplastic agent is paclitaxel.

In one embodiment, the combination of the present invention comprises an anti-OX40 ABP and a CTLA-4 modulator (e.g. anti-CTLA-4 ABP) and at least one anti-neoplastic agent which is a signal transduction pathway inhibitor.

In a further embodiment the signal transduction pathway inhibitor is an inhibitor of a growth factor receptor kinase VEGFR2, TIE2, PDGFR, BTK, erbB2, EGFr, IGFR-1, TrkA, TrkB, TrkC, or c-fms.

In a further embodiment the signal transduction pathway inhibitor is an inhibitor of a serine/threonine kinase rafk, akt, or PKC-zeta.

In a further embodiment the signal transduction pathway inhibitor is an inhibitor of a non-receptor tyrosine kinase selected from the src family of kinases.

In a further embodiment the signal transduction pathway inhibitor is an inhibitor of c-src.

In a further embodiment the signal transduction pathway inhibitor is an inhibitor of Ras oncogene selected from inhibitors of farnesyl transferase and geranylgeranyl transferase.

In a further embodiment the signal transduction pathway inhibitor is an inhibitor of a serine/threonine kinase selected from the group consisting of PI3K.

In a further embodiment the signal transduction pathway inhibitor is a dual EGFr/erbB2 inhibitor, for example N-{3-Chloro-4-[(3-fluorobenzyl) oxy]phenyl}-6-[5-({[2-(methanesulphonyl) ethyl]amino}methyl)-2-furyl]-4-quinazolinamine (structure below):

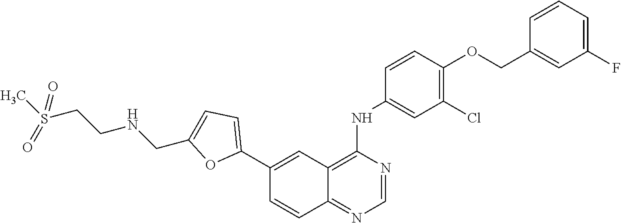 [SMILES: CS(=O)(=O)CCNCc1ccc(-c2ccc3ncnc(Nc4ccc(OCc5cccc(F)c5)c(Cl)c4)c3c2)o1]

In one embodiment, the combination of the present invention comprises a compound of formula I or a salt or solvate thereof and at least one anti-neoplastic agent which is a cell cycle signaling inhibitor.

In further embodiment, cell cycle signaling inhibitor is an inhibitor of CDK2, CDK4 or CDK6.

In one embodiment the mammal in the methods and uses of the present invention is a human.

As indicated, therapeutically effective amounts of the combinations of the invention (an anti-OX40 ABP and a CTLA-4 modulator (e.g. anti-CTLA-4 ABP)) are administered to a human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attendant physician.

The following examples are intended for illustration only and are not intended to limit the scope of the invention in any way.

### EXAMPLES

### Example 1

### Introduction

CT-26 is an N-nitroso-N-methylurethane-(NNMU) induced, undifferentiated murine colon carcinoma cell line in Balb/c mice. CT26 cells readily establish tumors in syngenic mice, producing histologically proven adenocarcinoma with a predictable growth rate in a reasonable time frame. In addition, CT26 tumor growth induced an extensive cellular immune response of predominantly regulatory T cells with a presumably protumoral activity. CT26 model has been widely used to evaluate the antitumor immune response of Immunotherapeutics. In these experiments, therapeutic use of the mouse analog of anti OX-40, OX-86, was evaluated in this mouse tumor model both independently and in combination with anti mouse CTLA-4 (9D9 clone)

### Methods

### Experimental Preparation(s)

All procedures on animals were reviewed and approved by the GSK Institutional Animal Care and Use Committee prior to initiation of the studies.

A frozen (−140° C.) vial of CT26, mouse colon carcinoma cells, from ATCC (cat# CRL-2638, lot#59227052) was thawed and the cells cultured according to the supplier's recommendations, approximately a week (three passages from thaw) before inoculation.

In the week prior to inoculation, 6-8 week old female Balb/c mice from Charles River were individually labelled via alpha-numerical tattoo on the tail or subcutaneously injected identification chip (BMDS, IMI-1000 transponder).

### Experimental Protocol(s)

The purpose of these experiments is to evaluate anti-tumor therapeutics in mouse syngeneic tumorgenesis models. Animals are weighed and inoculated on the right hind quarter with 100 ul of 0.5×105 CT26 tumor cells per mouse on Day 0. The number of mice inoculated is equal to 130% of what is needed for the study. Assuming 30% failure rate (either too big or too small at time of start of study), the goal is to have n=10 for each group. After tumor cell inoculation, tumor growth and total body weight are measured 3 times a week with a Fowler “ProMax” digital caliper for 4 weeks or longer. Antibodies were aquired from commercial vendor and diluted to desired concentration in 0.9% saline. Dosing (i.p.) occurs biweekly, for a total of 6 doses and initiates on the day of randomization when average tumor volume approximates 100 mm3, likely day 10 or 11. Randomization is performed using the Studylog Study Director Suite software. Length and width of tumors are measured in order to determine tumor volume using the formula (tumor volume=L×W2/2). Tumor measurement of greater than 2,000 mm3 for an individual animal results in euthanization. Mice may also be removed from the study due to weight loss (>20%), ulceration or tumor necrosis, or any other obvious inhibition of normal mouse activity due to morbidity.

Combination therapy; anti CTLA-4 (9D9) and OX86; N31299-11

For an experiment that was to include 10 groups of 10, 130 mice were inoculated. The purpose of this experiment was to repeat anti CTLA-4, OX-86 combination therapy results seen in N31299-1. Isotype controls for the high dose of OX-86 (400 ug of rat IgG1) and the high dose of anti CTLA-4 (100 ug of mouse IgG2b) were dosed individually and in combination. Monotherapies of 400 ug OX-86 and 100 ug of anti-CTLA-4 were tested with their respective appropriate isotype controls. A dose of 100 ug of anti CTLA-4 was evaluated in combination with 400, 100, 20, and 5 ug of OX-86. Statistical analysis of tumor volume was performed on day 19 (day 8 post randomization). Survivability analysis concludes at termination of the study on day 96.

| Dosing | treatment 1 | treatment 2 | n= |
|---|---|---|---|
| Group 1: 0.5 × 105 cells per, | saline | none | 10 |
| Group 2: 0.5 × 105 cells per, | RatIgG1 400 ug | none | 10 |
| Group 3: 0.5 × 105 cells per, | MouseIgG2b 100 ug | none | 10 |
| Group 4: 0.5 × 105 cells per, | RatIgG1 400 ug | MouseIgG2b 100 ug | 10 |
| Group 5: 0.5 × 105 cells per, | aCTLA-4 100 ug | RatIgG1 400 ug | 10 |
| Group 6: 0.5 × 105 cells per, | OX86 400 ug | MouseIgG2b 100 ug | 10 |
| Group 7: 0.5 × 105 cells per, | OX86 5 ug | aCTLA-4 100 ug | 10 |
| Group 8: 0.5 × 105 cells per, | OX86 20 ug | aCTLA-4 100 ug | 10 |
| Group 9: 0.5 × 105 cells per, | OX86 100 ug | aCTLA-4 100 ug | 10 |
| Group 10: 0.5 × 105 cells per, | OX86 400 ug | aCTLA-4 100 ug | 10 |

### Data Analysis

The event for survival analysis is tumor volume of 2000 mm3 or tumor ulceration, whichever came first. The exact time to cut-off volume was estimated by fitting a linear line between log tumor volume and day of two observations, the first observation that exceed the cut-off volume and the one observation that immediately preceded the cut-off volume. Kaplan-Meier (KM) method was carried out to estimate the survival probability of different treatment groups at a given time. The median time to endpoint and its corresponding 95% confidence interval was reported. Whether or not KM survival curves are statistically different between any two groups was then tested by log-rank test.

Tumor volume data from the last day in which there are 10 animals per gorup (i.e. before any animals are euthanized) were compared between the different treatment groups. Prior to the analysis, the tumor volume was natural log transformed due to the inequality of variance in the different treatment groups. ANOVA followed by pair-wise comparison were then carried out on the log transformed data

Combination therapy; anti CTLA-4 (9D9) and OX86

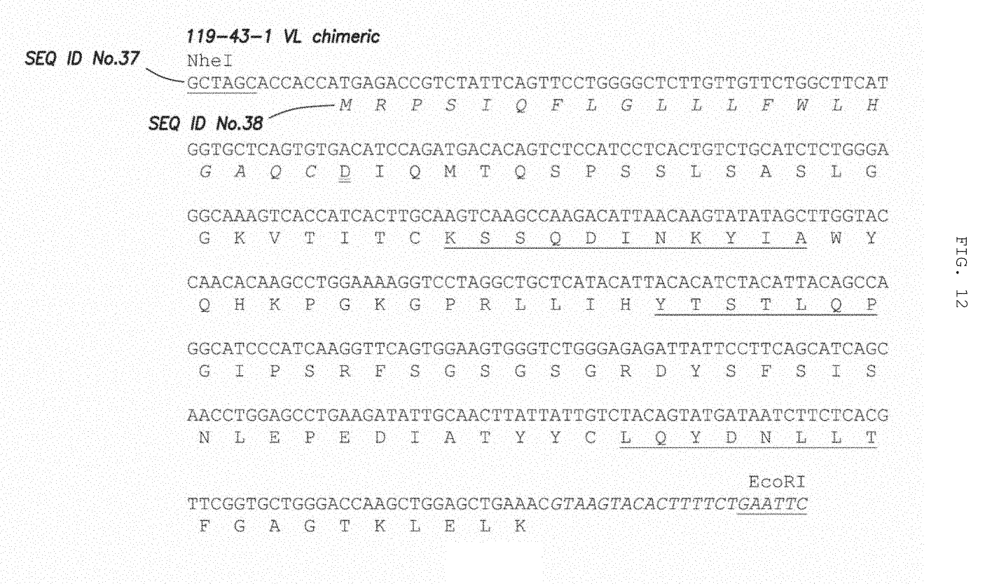

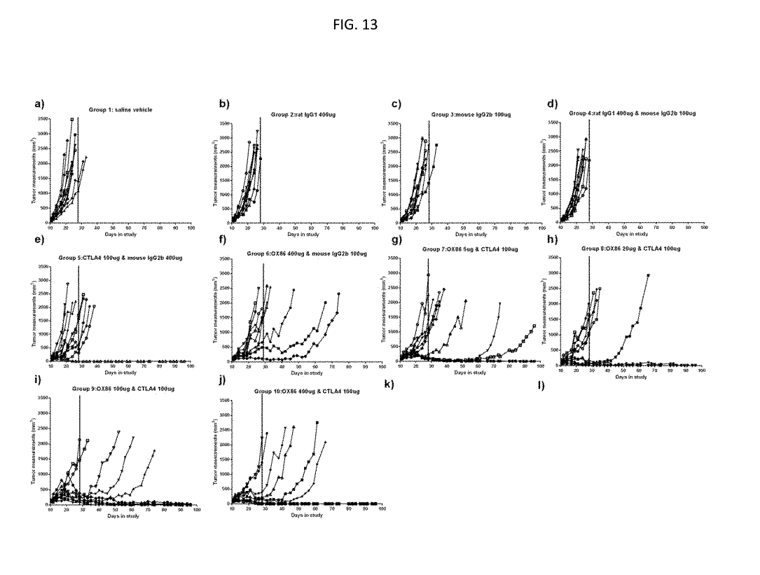

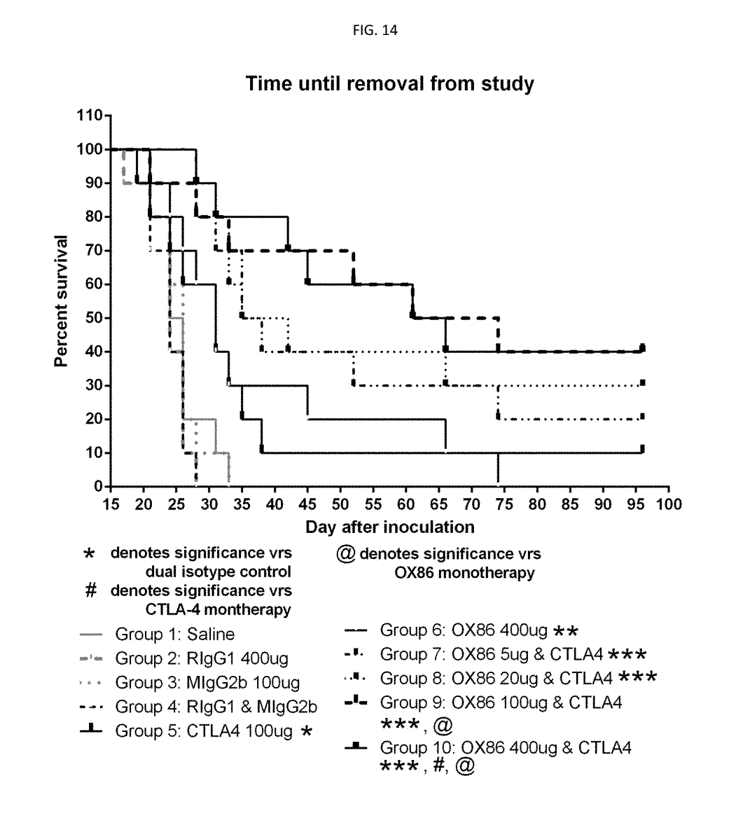

Balb/c mice that were inoculated with 0.5×105 CT-26 colorectal tumor cells and dosed i.p. with sterile saline or antibody isotype controls developed tumors that progressed in size as expected (FIG. 13, panel a). In this experiment, isotype controls were evaluated individually in addition to as a combination. There was no significant decrease in tumor volume at day 19 between saline and isotype controls, or between isotype monotherapies and the isotype dual therapy (FIG. 13, panels b,c,d). The same holds true for differences in survivability among these groups. Treatment with 100 ug of anti-CTLA-4 resulted in no significant delay in tumor growth rate at day 19 when compared to saline and isotype controls, however, the monotherapy did result in an increase in survivability when compared to the dual isotype control (p<0.05) (FIG. 14). One animal in the CTLA-4 monotherapy group lost its tumor entirely and remained tumor free through study termination (FIG. 13, panel e). The 400 ug OX86 monotherapy reduced average tumor volume at day 19 and increased survivability when compared to saline control (p<0.05 for both) and isotype control (p<0.05 and p<0.01, respectively) groups (data not shown). The 400 ug monotherapy of OX86 resulted in an observable delay in tumor growth in three mice relative to isotype controls (FIG. 13, panel f). When combined with 100 ug CTLA-4, OX86 demonstrated the ability to reduce average tumor growth when compared to saline and dual isotype controls at 5 ug (p<0.05, p<0.05), 20 ug (p<0.01, p<0.05), 100 ug (p<0.05, p<0.05), and 400 ug (p<0.01, p<0.01) (data not shown). At study termination (day 96), a total of 11 mice had totally regressed tumors in combination groups; two in the 5 ug OX86/CTLA-4 group (FIG. 13, panel g), 3 in the 20 ug OX86/CTLA-4 (FIG. 13, panel h), 2 in the 100 ug OX86/CTLA-4 (FIG. 13, panel i), and 4 in the 400 ug OX86/CTLA-4 group (FIG. 13, panel j). In addition to this, the 100 ug OX86/CTLA-4 combo group had two animals in which the tumors had regressed to smaller than their size at their inclusion into the study. A drastic increase in survivability was seen in all combination therapy doses when compared to saline and isotype controls (p<0.001 for all) (FIG. 14). A total of 5 out of 100 animals (5%) had had to be removed from the study due to ulceration. One animal group 2 (400 ug rat IgG1) was found dead in its cage by LAS staff.

As shown in FIG. 14: Mice were inoculated with 0.5e5 CT-26 cells per mouse on day 0. Animals were randomized and dosing began on day 11 after inoculation and continued twice a week for 3 weeks (total of 6 doses). Vehicle control group was dosed i.p. with saline and the isotype control received i.p. doses of the isotypes for OX86 (rat IgG1) and anti CTLA-4 9D9 (mouse IgG2b), both individually and in combination. Monotherapy animals were dosed with either 400 ug OX86 and 100 ug mouse IgG2b or 100 ug of anti CTLA-4 and 400 ug of rat IgG1. Combination therapy mice recieved 100 ug of anti CTLA-4 and either 5, 20, 100, or 400 ug of OX86. Tumor growth was analyzed at day 19 post inoculation where all combination therapies demonstrated a significant decrease in average tumor size relative to isotype control. All combination therapies demonstrated a significant increase in survivability when compared to the isotype control. The 100 and 400 ug OX86 combination therapy groups also whitnessed a significant in crease in survivability when compared to OX86 monotherapy and the 400 ug combination group against the CTLA-4 monotherapy. (*p<0.05, **p<0.01, ***p<0.001; ANOVA followed by pair-wise comparison were carried out on natural log transformed tumor volume data, log-rank test was used to determine significance in KM survival curves).

Conclusion: In conclusion, in the CT26 syngeneic model. OX86 combinations with anti-CTLA-4 also resulted in significantly increased survival compared to OX86 and CTLA-4 monotherapies.

### Example 2

### OX-401 CTLA-4 CT-26 Rechallenge Study

### Methods

A frozen (−140° C.) vial of CT26, mouse colon carcinoma cells, from ATCC (cat# CRL-2638, lot#59227052) cells were thawed and cultured in basic RPMI (with 10% FBS) media over the following week. Cells (passage 5 post thaw) were harvested from the flask in complete medium. Cells were centrifuged and resuspended in RPMI (with 10% FBS). This step was repeated 3 times in RPMI media without FBS. Cell density and viability were checked via trypan blue exclusion either through the use of hemocytometer or Vicell. Cells were then diluted to desired density (5×10⁵ cells per mL) for inoculation.

Female balb/c mice, aged 6 to 7 weeks, that had been previously injected subcutaneously between the shoulders with IMI-1000 transponders were inoculated with 100 ul of the 5×10⁵ cells per mL suspension. Injections were SC on the right hind quarter of the mouse with a 25G needle. A total of 130 mice were inoculated with tumor cells. Assuming 30% failure rate (either too big or too small at time of start of study), the goal was to have n=10 for each group. The number of mice per group was doubled for combination therapy groups so as to increase the total number of mice with regressed tumors. An additional 13 mice were left uninoculated until the time of re-challenge to serve as an age matched control. After tumor cell inoculation, tumor growth and total body weight were measured 3 times a week throughout the study. Randomization occured on day 13 post inoculation when average tumor size was approximately 100 mm³. Dosing (i.p.), beginning on day 14 post inoculation, occurred biweekly for a total of 6 doses. Mice remained on study until tumors reached >2000 cu mm or until the end of the study. Mice may also have been removed from the study due to weight loss (>20%), ulceration or tumor necrosis, or any other obvious inhibition of normal mouse activity due to morbidity. On day 44 post inoculation, all mice remaining on study with measurable tumors were euthanized. On day 71 post inoculation, all remaining mice with completely regressed tumors, along with the 13 naive mice were inoculated on the left flank with 0.5×10⁵ CT-26 cells using the same method described above.

Tumor growth was measured 3 times a week with a Fowler “ProMax” digital caliper. Randomization was performed using the Studylog Study Director Suite software. Length and width of tumors were measured in order to determine tumor volume using the formula (tumor volume=L×W²×0.52). Tumor measurement of greater than 2,000 mm³ for an individual animal resulted in euthanization for that animal.

All Abs (antibodies) were diluted to desired concentrations in 0.9% sodium chloride (Hospira, NDC 0409-4888-10, lot #44-324-DK) on the day of dosing and injected i.p. in a volume of 0.2 mL per mouse using a 30G needle. For this experiment, OX-86, CTLA-4 and their respective isotype controls were dosed at 100 ug per mouse. The mice receiving the OX86 monotherapy also received the isotpye control for CTLA-4, mouse IgG2b. Similarly, the mice being dosed with the CTLA-4 monotherapy were also dosed with the isotpye control for OX86, rat IgG1. The isotype group received both isotype controls and served as an isotype control. The vehicle control mice were dosed with 0.9% saline.

| | | | | Conc. |
|---|---|---|---|---|
| Antibody | Supplier | Cat # | Lot # | (mg/mL) |
| Rat IgG1 | Bioxcell | BE0088 | 5339/0814 | 7.86 |
| OX86 | Bioxcell | BE0031 | 5534/01214 | 7.75 |
| (Hybridoma | | | | |
| CD134) | | | | |
| Mouse IgG2b | Bioxcell | BE0086 | 4700/1014 | 4.46 |
| Anti-m CTLA-4 | Bioxcell | BE0164 | 5338/1214 | 5.97 |
| (9D9) | | | | |
| Ipilimumab | BMS/ | NDC0003- | LotAAA3262 | 5 mg/mL |
| | Bio- | 2327-11 | | |
| | compare | | | |

Evaluating Pharmacodynamic changes in markers: Female BALB/c (8-10 wks old from Charles River Laboratories) mice were implanted with 0.5×105 CT-26 colon carcinoma cells (ATCC #CRL-2638) subcutaneously on their flanks. Mice were randomized and treatment was started when the tumors reached approximately 100 mm³. The mice were randomized into five groups receiving vehicle, isotype control (IgG1 100 ug (micrograms)+IgG2b 100 ug), anti-OX40 (OX86 100 ug+IgG2b 100 ug), anti-CTLA-4 (CTLA-4 200 ug+IgG1 100 ug) or anti-OX40 and anti-CTLA-4 (OX86100u+CTLA-4 100 ug) The mice were dosed twice a week and harvested on day 3, 7 and 10 following the first dose. For e.g mice collected on day 3, 7 and 10 received 1, 2 and 3 doses respectively. Serum was collected for cytokine analysis and analyzed by Meso-Scale Discovery mouse V-flex customized kit. Blood and tumor samples were collected, following dissociation of the tumors both the tissues were stained with antibodies for flow cytometric analysis and data was acquired on BD FACSCanto II. The data were analyzed by FlowJo software and statistical analysis was carried out by one way Anova using Kruskal-Wallis test. Gene expression in tumors was analyzed by NanoString technology.

### Tumor Isolation:

Excised tumors were mechanically dissociated in a 60 mm petri dish on ice with 2 mL of PBS and were transferred to 5-15 mL polypropylene tubes. Miltenyi Tumor Dissociation Kit cocktail of enzymes was prepared in RPMI as per the kit protocol and added to each tube (˜2.5 mL per tumor). Digestion was allowed to proceed for 40 min at 37° C. After digestion, the remaining pieces and the suspension were filtered through 30-100 μm cell strainers into a 50 mL tube. The strainers were washed with 10 mL PBS and brought up to 20 mL. Tubes containing cells were centrifuged at 300 rcf for 5 min, supernatant aspirated and pellet resuspended in 5 mL PBS. Lymphocytes and tumor cells were counted and total cell number was estimated using Vi-CELL. Cells were diluted to a final concentration of 1×10⁶ cell/100 uL (microliters) and submitted stained with antibodies.

### Flow Cytometric Analysis:

400 ul of blood sample was transferred into 8 mL ACK lysis buffer in a 15 mL conical tube, mixed well and incubated at room temperature for 5 min. Tubes were centrifuged at 3500×g for 10 min at 10° C. and supernatant aspirated. Pellet was resuspended in 10 mL of FACS buffer and centrifuged at 2700×g for 5 min at 10° C. Supernatants were aspirated, pellets resuspended in 10 mL FACS buffer and tubes spun at 1600 rpm (IEC CL31R) for 5 min at 10° C. The wash was repeated with FACS buffer. Cell pellets were resuspended in 800 μl FACS buffer. 100 ul cell suspension of either cells isolated from tumor, spleen or blood were aliquoted into each 96-well plate, and the plate was centrifuged at 1600 rpm (IEC CL31R) for 5 min. The supernatants were removed. Cells were blocked with 50 ul of FACS buffer plus 2% rat serum or FcR Blocking Reagent for 10 min on ice. Cells were stained by adding desired dilution of antibodies in 50 ul of FACS buffer and incubating on ice for 30 minutes. Intracellular staining was carried out by adding 200 uL of Foxp3 Fixation/Permeabilization working solution was added to each well, and cells were resuspended. Cells were incubated in the dark at 4° C. for 30 minutes. Plates were centrifuged at room temperature for 5 minutes at 400×g, and the supernatant was discarded. Cells were washed twice in 200 uL of 1× Permeabilization Buffer was added to each well. Cells were blocked with 2% rat serum/1% hamster serum and incubated at room temperature for 15 minutes. Without washing, the recommended amount of fluorochrome-conjugated antibody was added for detection of intracellular antigen(s), and plates were incubated in the dark at room temperature for at least 30 minutes. Cells were washed twice in 200 μL of 1× Permeabilization Buffer. Stained cells were resuspended in an appropriate volume of Flow Cytometry Staining Buffer and stored at 4° C. until acquisition on the flow cytometer the following day.

### Mouse T-Cell Receptor Sequencing:

Female BALB/c (8-10 wks old from Charles River Laboratories) mice were implanted with 0.5×10⁵ CT-26 colon carcinoma cells (ATCC #CRL-2638) subcutaneously on their flanks. Mice were randomized and treatment was started when the tumors reached approximately 100 mm³. The mice were randomized into four groups receiving isotype controls (isotype (IgG1 100 ug+IgG2b 100 ug), anti-OX40 (OX86 100 ug+IgG2b 200 ug), anti-CTLA4 (CTLA4 100 ug+IgG1 100 ug) or anti-OX40 and anti-CTLA4 (OX86100 ug+CTLA4 100 ug). antibodies. Each group had 7 mice, the mice were dosed twice a week, 72 hours post the 3^{rd} dose the mice were euthanized, blood was collected in EDTA blood collection tubes (BD Microtainer 365974), while the tumors were collected in 1.8 ml cryotubes flash frozen and shipped to Adaptive Biotechnologies for TCRI3 analysis.

### In Vitro Studies:

Healthy donor PBMCs were isolated then stimulated suboptimally using anti CD3/anti CD28 T cell Expander beads at a bead to cell ratio of 1:20. Cells were seeded at 1×10̂6 cells/mL in AIM-V medium supplemented with 100 IU/ml of rhIL-2 and 100 ng/ml of MCSF and cultured for 2 days at 37° C. The beads were magnetically removed and the cells were seeded in a 96well plate. Then re-stimulated with anti CD3 beads at a bead to cell ratio of 1:1 together with the addition of anti OX40 and ipilimumab (CTLA-4) 10 ug/mL. The supernatants were collected after 96 hrs and analysis was performed using Meso Scale Discovery forIFN-γ and TNF-α.

### PD Data (CT-26)

Serum cytokine levels were tested using the Meso-Scale Discovery mouse V-flex customized kit. Samples and calibrators were diluted 1:2 in Diluent 41 as per the kit manual. 50 ul of prepared samples and calibrators were added to the MSD plate each in triplicate. Plates were sealed and incubated at room temperature with shaking for 2 hours. Plates were washed 3 times with 150 u/well of PBS plus 0.05% Tween-20. 25 uL of detection antibody solution prepared in Diluent 45 was added to each well. Plates were sealed and incubated at room temperature with shaking for 2 hours. Plates were washed as above. 150 ul/well of freshly diluted 2× read buffer was added to the plates which were immediately read on MESO QuickPlex reader. Data were analyzed using MSD Workbench software and graphed using GraphPad Prism.

### Conclusions:

### Re-Challenges Study in CT-26 Model

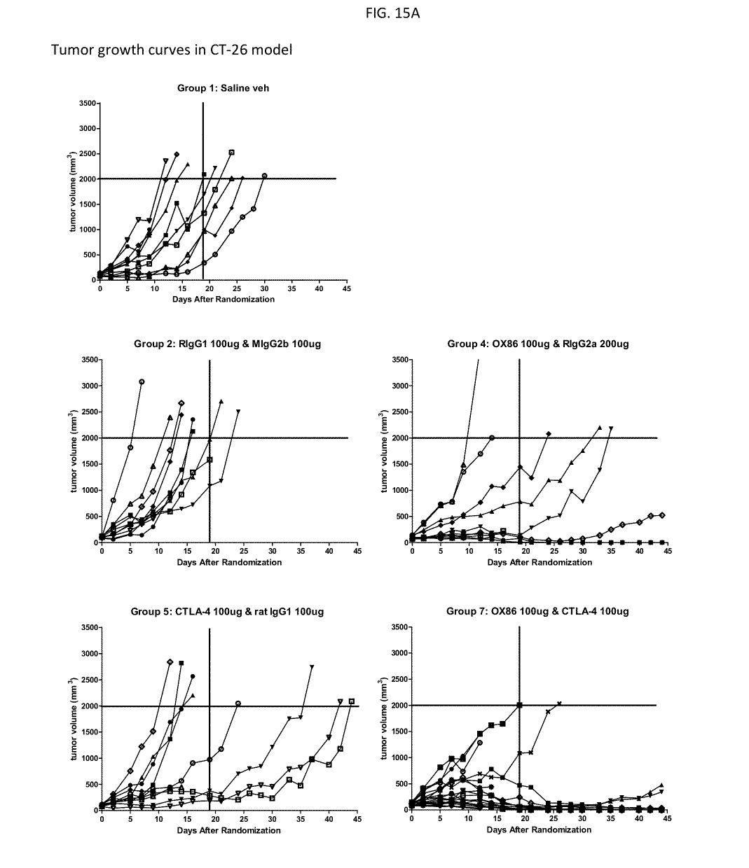

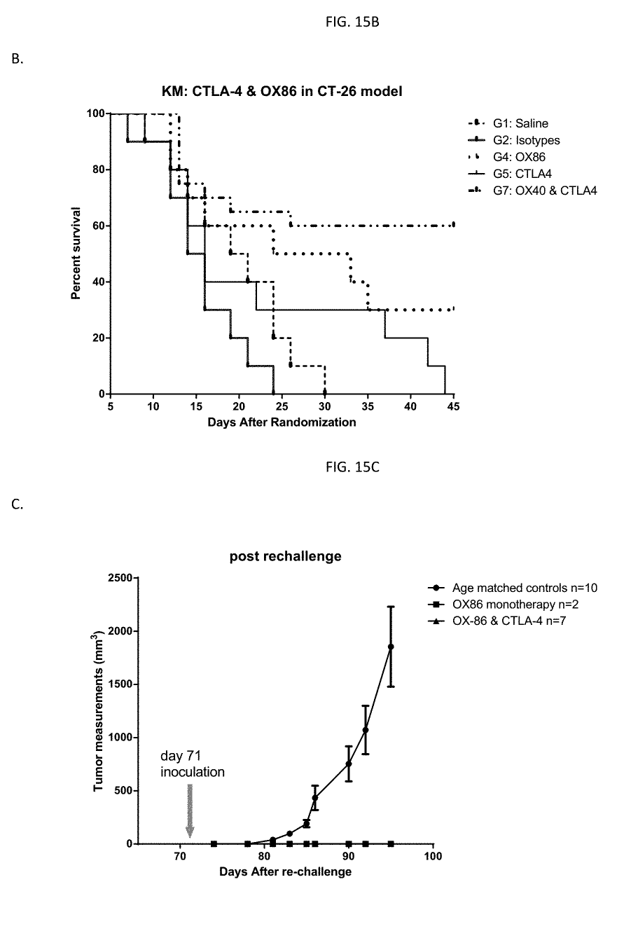

Combination therapies resulted in higher tumor regression rate and survivability than that seen in monotherapies (FIG. 15A)
2 out of 10 in OX86 monotherapy group
7 out of 20 in CTLA-4/OX86 treatment group
Mice cured with either OX86 or OX86/CTLA4 are completely protected from CT-26 tumor rechallenge as demonstrated by total tumor loss compared to control group (FIG. 15B and FIG. 15C)

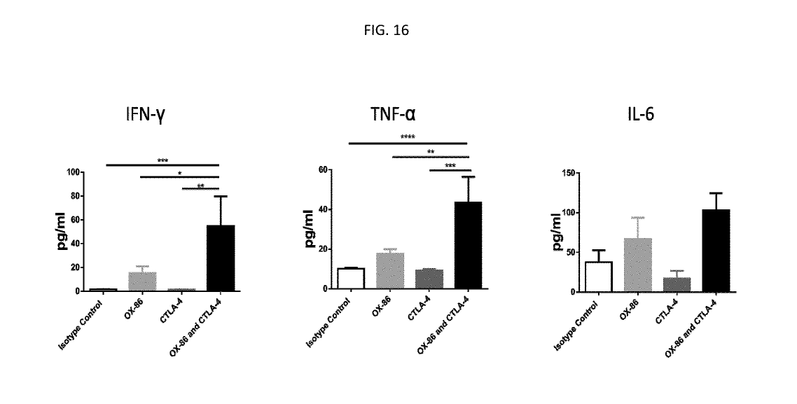

PD Data Cytokine Analysis:
a. Th1 cytokines IFNγ and TNFα are significantly upregulated in the group receiving both anti-OX40 and anti-CTLA-4 treatment. Similar trends are also observed with IL6 though the differences are not statistically significant. (FIG. 16)

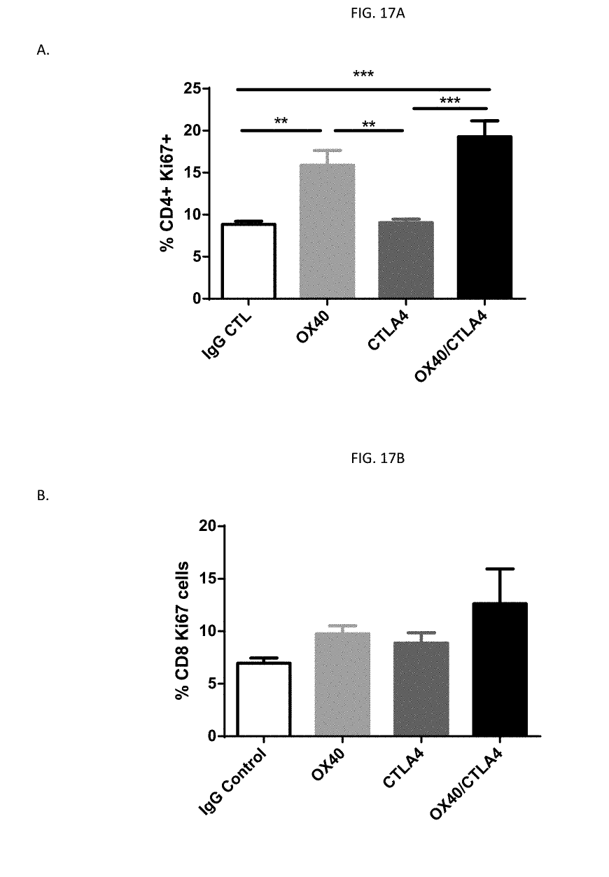

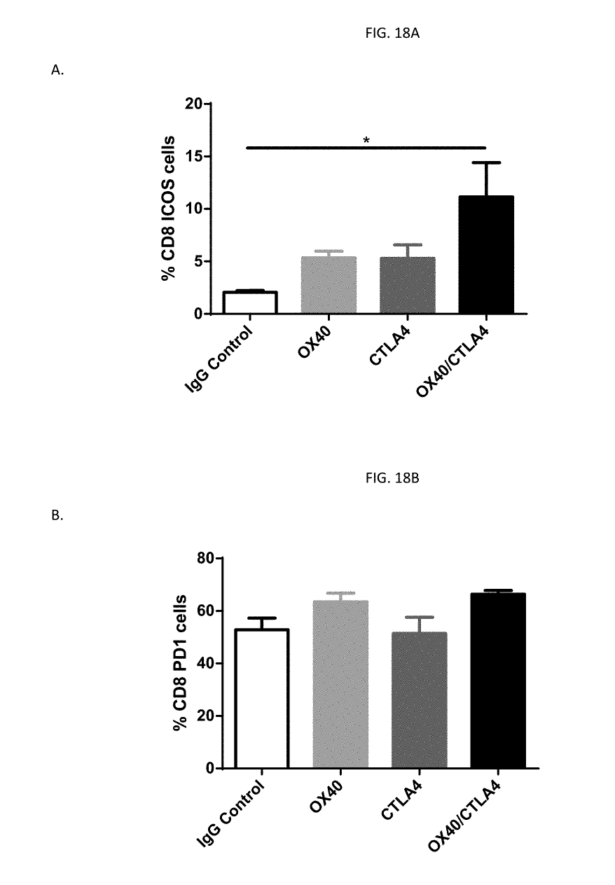

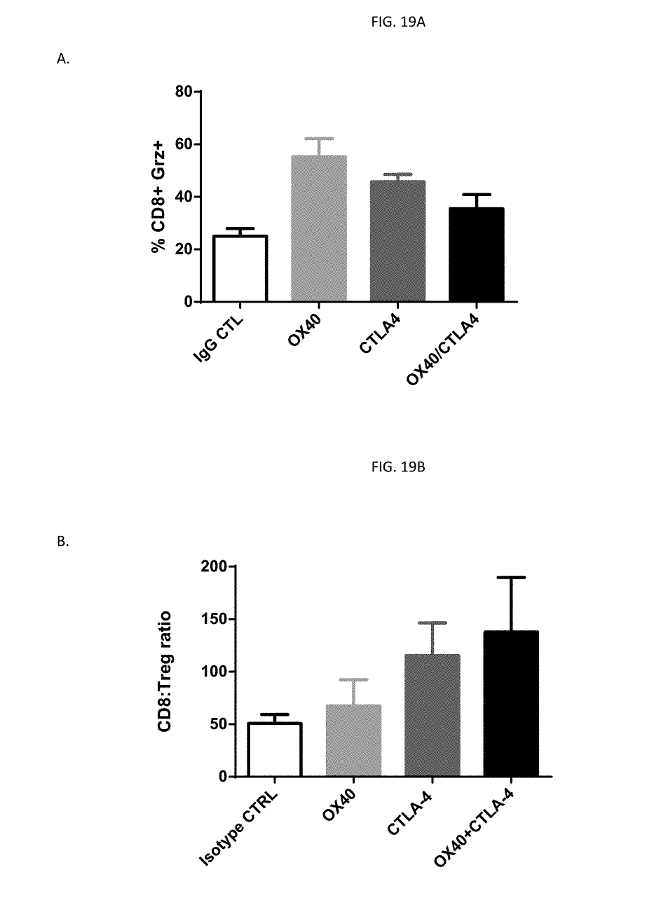

PD Immunophenotyping Analysis:
a. Anti-OX40 treatment either as a monotherapy or as a combination with anti-CTLA-4 treatment significantly increased the proliferation of CD4 T cells in the spleen, as measured by Ki67+ staining (FIG. 17A). The combination treatment also increased CD8 T cell numbers in the spleen (FIG. 17B).
b. There was a significant increase in the activated (CD25^{+ive}) CD4T-cells in the spleen following anti-OX40 and anti-CTLA-4 treatment.
c. There was a significant increase in the activated (CD25^{+ive}) CD4 and CD8 T-cells in the tumors following anti-OX40 and anti-CTLA-4 treatment.
d. There was a significant increase in the ICOS expressing CD8 T cells in the group treated with the combination antibodies (FIG. 18A). An increased trend was observed but was not significant in PD1 expressing CD8 T cells treated with anti-OX40 monotherapy or the combination (FIG. 18B).
e. While not statistically significant, increases in Granzyme B and CD8:Treg ratios were observed with anti-OX40 or combination treatment in tumors. (FIG. 19A and FIG. 19B)

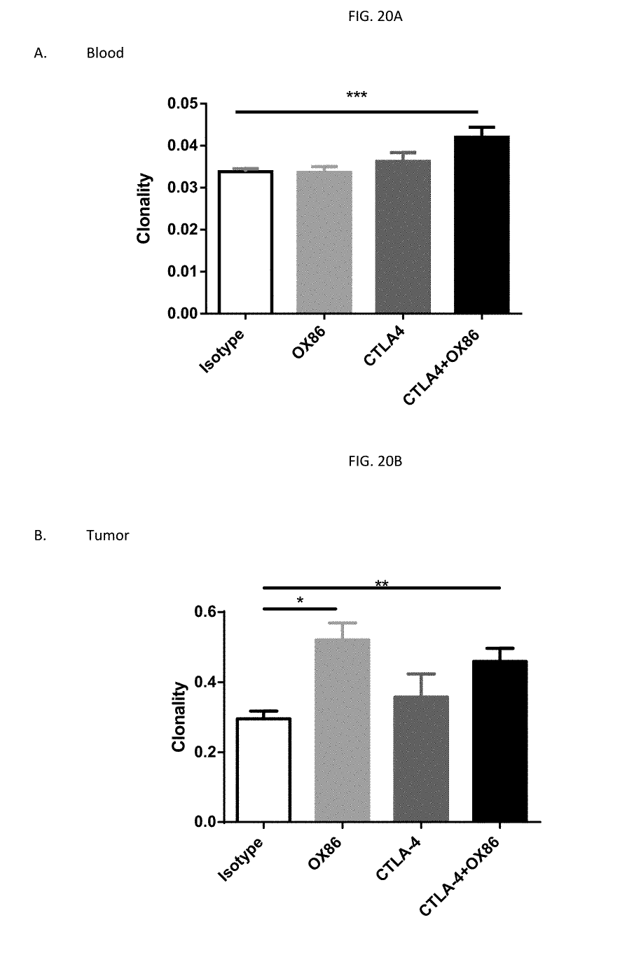

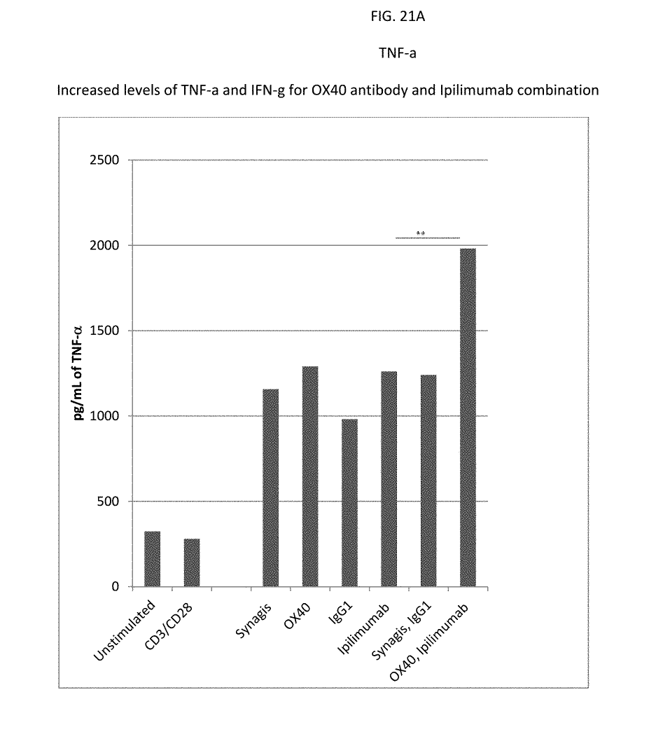

Pd Tcrβ Analysis:
a. There is a significant increase in clonality of TCR repertoire in the blood following treatment with anti-OX40 and anti-CTLA-4 combination treatment (FIG. 20A). However, in the tumor both anti-OX40 monotherapy as well as combination significantly increases clonality of the TCR repertoire (FIG. 20B).

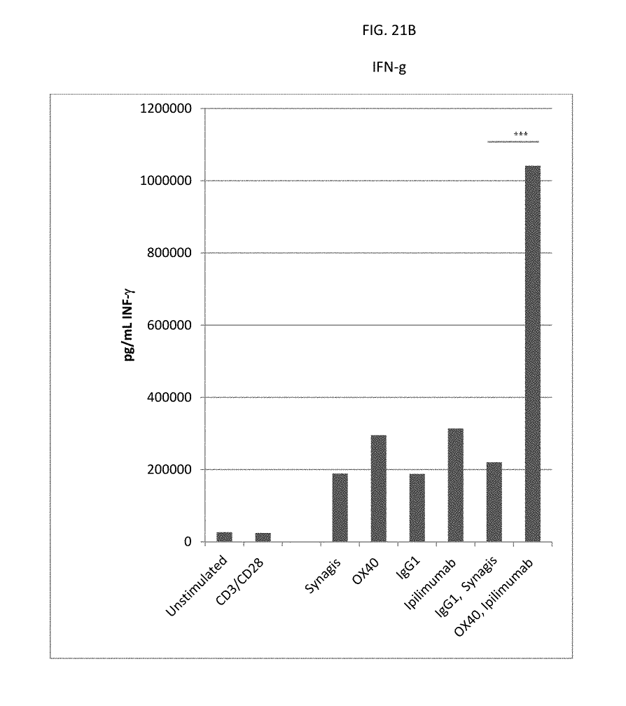

In Vitro Assays:
OX40 agonism synergizes with anti-CTLA4 antagonism to enhance T cell function by increasing IFN-g and TNF-a production. (FIG. 21A and FIG. 21B)

1. A method of treating cancer in a mammal in need thereof comprising administering to the mammal a therapeutically effective amount of an antigen binding protein that binds OX40 and an antigen binding protein that binds CTLA-4.

2. The method of claim 1, wherein the cancer is a solid tumor.

3. The method of claim 1, wherein the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

4. The method of claim 1, wherein the cancer is a liquid tumor.

5. The method of claim 1, wherein the antigen binding protein that binds OX40 and the antigen binding protein that binds CTLA-4 are administered at the same time.

6. The method of claim 1, wherein the antigen binding protein that binds OX40 and the antigen binding protein that binds CTLA-4 are administered sequentially, in any order.

7. The method of claim 1, wherein antigen binding protein that binds OX40 and/or the antigen binding protein that binds CTLA-4 are administered systemically.

8. The method of claim 1, wherein the antigen binding protein that binds OX40 and/or the antigen binding protein that binds CTLA-4 is administered intratumorally.

9. The method of claim 1, wherein the mammal is human.

10. The method of claim 1, wherein the tumor size of said cancer in said mammal is reduced by more than an additive amount compared with treatment with the antigen binding protein to OX40 and the antigen binding protein to CTLA-4 as used as monotherapy.

11. The method of claim 1, wherein the antigen binding protein that binds OX40 binds to human OX40.

12. The method of claim 1, wherein the antigen binding protein that binds to CTLA-4 binds to human CTLA-4.

13. The method of claim 1, wherein the antigen binding protein that binds OX40 and/or the antigen binding protein that binds CTLA-4 is a humanized monoclonal antibody.

14. The method of claim 1, wherein the antigen binding protein that binds OX40 and/or the antigen binding protein that binds CTLA-4 is a fully human monoclonal antibody.

15. The method of claim 1, wherein the antigen binding protein that binds OX40 and/or the antigen binding protein that binds CTLA-4 is an antibody with an IgG1 antibody isotype or variant thereof.

16. The method of claim 1, wherein the antigen binding protein that binds OX40 and/or the antigen binding protein that binds CTLA-4 is an antibody with an IgG4 antibody isotype or variant thereof.

17. The method of claim 1, wherein the antigen binding protein that binds OX40 is an agonist antibody.

18. The method of claim 1, wherein the antigen binding protein that binds CTLA-4 is an antagonist antibody.

19. The method of claim 1, wherein the antigen binding protein that binds OX40 comprises: a heavy chain variable region CDR1 comprising an amino acid sequence with at least 90%, 91%, 92%, 93%, 94%, 95%, 96%, 97%, 98%, 99% or 100% sequence identity to the amino acid sequence set forth in SEQ ID NO:1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO:2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:3 or 15.

20. The method of claim 1, wherein the antigen binding protein that binds OX40 comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO:7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO:8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:9 or 21.

21. The method of claim 1, wherein the antigen binding protein that binds OX40 comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

22. The method of claim 1, wherein the antigen binding protein that binds OX40 comprises: (a) a heavy chain variable region CDR1 comprising the amino acid sequence of SEQ ID NO:13; (b) a heavy chain variable region CDR2 comprising the amino acid sequence of SEQ ID NO:14; (c) a heavy chain variable region CDR3 comprising the amino acid sequence of SEQ ID NO:15; (d) a light chain variable region CDR1 comprising the amino acid sequence of SEQ ID NO:19; (e) a light chain variable region CDR2 comprising the amino acid sequence of SEQ ID NO:20; and (f) a light chain variable region CDR3 comprising the amino acid sequence of SEQ ID NO:21.

23. The method of claim 1, wherein the antigen binding protein that binds OX40 comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:10, 11, 22 or 23.

24. The method of claim 1, wherein the antigen binding protein that binds OX40 comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:4, 5, 16 and 17.

25. The method of claim 1, wherein the antigen binding protein that binds OX40 comprises a heavy chain variable region comprising the amino acid sequence set forth in SEQ ID NO:5 and a light chain variable region comprising the amino acid sequence set forth in SEQ ID NO:11.

26. The method of claim 1, wherein the antigen binding protein that binds OX40 comprises a heavy chain variable region comprising the amino acid sequence set forth in SEQ ID NO:17 and a light chain variable region comprising the amino acid sequence set forth in SEQ ID NO:23.

27. The method of claim 1, wherein the antigen binding protein that binds OX40 comprises a light chain variable region comprising the amino acid sequence of SEQ ID NO:11 or 23, or an amino acid sequence with at least 90% sequence identity to the amino acid sequences of SEQ ID NO:11 or 23.

28. The method of claim 1, wherein the antigen binding protein that binds OX40 comprises a heavy chain variable region comprising the amino acid sequence of SEQ ID NO:5 or 17, or an amino acid sequence with at least 90% sequence identity to the amino acid sequences of SEQ ID NO:5 or 17.

29. The method of claim 1, wherein the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

30. The method of claim 1, wherein the antigen binding protein that binds CTLA-4 is ipilimumab, or an antibody comprising 90%, 91%, 92%, 93%, 94%, 95%, 96%, 97%, 98%, 99% or 100% sequence identity thereto.

31. The method of claim 1, wherein the antigen binding protein that binds CTLA-4 is tremelimumab, or an antibody having 90%, 91%, 92%, 93%, 94%, 95%, 96%, 97%, 98%, 99% or 100% sequence identity thereto.

32. The method of claim 1, wherein the mammal has increased survival when treated with a therapeutically effective amount of an antigen binding protein to OX40 and therapeutically effective amount of an antigen binding protein to CTLA-4 compared with a mammal who received the antigen binding protein to OX40 or the antigen binding protein to CTLA-4 as monotherapy.

33. The method of claim 1, further comprising administering at least one anti-neoplastic agent to the mammal in need thereof.

34. A pharmaceutical composition or kit comprising a therapeutically effective amount of an antigen binding protein that binds OX40 and a therapeutically effective amount of an antigen binding protein that binds CTLA-4.

35.-41. (canceled)

42. A method of reducing tumor size in a human having cancer comprising administering a therapeutically effective amount of an agonist antibody to human OX40 and a therapeutically effective amount of an antagonist antibody to human CTLA-4.
